SEQUENCE LISTING (25 sequences)

SEQ ID NO: 1 - DNA, 1365 bp, Artificial Sequence
  Nucleotide Sequence encoding TNFR1
atgggcctctccaccgtgcctgacctgctgctgccgctggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccccagattgagaatgttaagggcactgaggactcaggcaccacagtgctgttgcccctggtcattttctttggtctttgccttttatccctcctcttcattggtttaatgtatcgctaccaacggtggaagtccaagctctactccattgtttgtgggaaatcgacacctgaaaaagagggggagcttgaaggaactactactaagcccctggccccaaacccaagcttcagtcccactccaggcttcacccccaccctgggcttcagtcccgtgcccagttccaccttcacctccagctccacctatacccccggtgactgtcccaactttgcggctccccgcagagaggtggcaccaccctatcagggggctgaccccatccttgcgacagccctcgcctccgaccccatccccaacccccttcagaagtgggaggacagcgcccacaagccacagagcctagacactgatgaccccgcgacgctgtacgccgtggtggagaacgtgcccccgttgcgctggaaggaattcgtgcggcgcctagggctgagcgaccacgagatcgatcggctggagctgcagaacgggcgctgcctgcgcgaggcgcaatacagcatgctggcgacctggaggcggcgcacgccgcggcgcgaggccacgctggagctgctgggacgcgtgctccgcgacatggacctgctgggctgcctggaggacatcgaggaggcgctttgcggccccgccgccctcccgcccgcgcccagtcttctcaga

SEQ ID NO: 2 - protein, 455 aa, Artificial Sequence
  Amino acid sequence of TNFR1
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 3 - DNA, 591 bp, Artificial Sequence
  Nucleotide sequence encoding a Ligand Binding Domain of TNFR1
atgggcctctccaccgtgcctgacctgctgctgccgctggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctacca

SEQ ID NO: 4 - protein, 197 aa, Artificial Sequence
  Amino acid sequence of a Ligand Binding Domain of TNFR1
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLP

SEQ ID NO: 5 - DNA, 1008 bp, Artificial Sequence
  full-length FAS
atgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaagagaaaggaagtacagaaaacatgcagaaagcacagaaaggaaaaccaaggttctcatgaatctccaactttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctag

SEQ ID NO: 6 - protein, 335 aa, Artificial Sequence
  full-length FAS
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 7 - DNA, 505 bp, Artificial Sequence
  Effector Domain of FAS
aggatccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctag

SEQ ID NO: 8 - protein, 167 aa, Artificial Sequence
  Effector Domain of FAS
GSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 9 - DNA, 1101 bp, Artificial Sequence
  FAS-chimera
atgggcctctccaccgtgcctgacctgctgctgccgctggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccaagcttaggatccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctag

SEQ ID NO: 10 - protein, 366 aa, Artificial Sequence
  FAS-chimera
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPSLGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 11 - DNA, 47 bp, Artificial Sequence
  cis-regulatory element
ctggagggtgactttgcttctggagccagtacttcatacttttcatt

SEQ ID NO: 12 - DNA, 47 bp, Artificial Sequence
  cis-regulatory element
aatgaaaagtatgaagtactggctccagaagcaaagtcaccctccag

SEQ ID NO: 13 - DNA, 44 bp, Artificial Sequence
  cis-regulatory element
gtacttcatacttttcattccaatggggtgactttgcttctgga

SEQ ID NO: 14 - DNA, 44 bp, Artificial Sequence
  cis-regulatory element
tccagaagcaaagtcaccccattggaatgaaaagtatgaagtac

SEQ ID NO: 15 - DNA, 131 bp, Artificial Sequence
  cis-regulatory element
ctccagaagcaaagtcaccccattggaatgaaaagtatgaagtacaatgaaaagtatgaagtactggctccagaagcaaagtcaccctccagaagcaaagtcaccccattggaatgaaaagtatgaagtac

SEQ ID NO: 16 - DNA, 131 bp, Artificial Sequence
  cis-regulatory element
gtacttcatacttttcattccaatggggtgactttgcttctggagggtgactttgcttctggagccagtacttcatacttttcattgtacttcatacttttcattccaatggggtgactttgcttctggag

SEQ ID NO: 17 - DNA, 850 bp, Artificial Sequence
  Endothelial Cell-Specific PPE-1 Promoter
gtacgtgtacttctgatcggcgatactagggagataaggatgtgcctgacaaaaccacattgttgttgttatcattattatttagttttccttccttgctaactcctgacggaatctttctcacctcaaatgcgaagtactttagtttagaaaagacttggtggaaggggtggtggtggaaaagtagggtgatcttccaaactaatctggttccccgcccgccccagtagctgggattcaagagcgaagagtggggatcgtccccttgtttgatcagaaagacataaaaggaaaatcaagtgaacaatgatcagccccacctccaccccacccccctgcgcgcgcacaatacaatctatttaattgtacttcatacttttcattccaatggggtgactttgcttctggagaaactcttgattcttgaactctggggctggcagctagcaaaaggggaagcgggctgctgctctctgcaggttctgcagcggtctctgtctagtgggtgttttctttttcttagccctgcccctggattgtcagacggcgggcgtctgcctctgaagttagccgtgatttcctctagagccgggtcttatctctggctgcacgttgcctgtgggtgactaatcacacaataacattgtttagggctggaatgaagtcagagctgtttacccccactctataggggttcaatataaaaaggcggcggagaactgtccgagtcagaagcgttcctgcaccggcgctgagagcctgacccggtctgctccgctgtccttgcgcgctgcctcccggctgcccgcgacgctttcgccccagtggaagggccacttgctgcggccgc

SEQ ID NO: 18 - DNA, 987 bp, Artificial Sequence
  Endothelial Cell-Specific PPE-1 3X Promoter
gtacgtgtacttctgatcggcgatactagggagataaggatgtgcctgacaaaaccacattgttgttgttatcattattatttagttttccttccttgctaactcctgacggaatctttctcacctcaaatgcgaagtactttagtttagaaaagacttggtggaaggggtggtggtggaaaagtagggtgatcttccaaactaatctggttccccgcccgccccagtagctgggattcaagagcgaagagtggggatcgtccccttgtttgatcagaaagacataaaaggaaaatcaagtgaacaatgatcagccccacctccaccccacccccctgcgcgcgcacaatacaatctatttaattgtacttcatacttttcattccaatggggtgactttgcttctggagaaactcttgattcttgaactctggggctggcagctagcctccagaagcaaagtcaccccattggaatgaaaagtatgaagtacaatgaaaagtatgaagtactggctccagaagcaaagtcaccctccagaagcaaagtcaccccattggaatgaaaagtatgaagtacgctagcaaaaggggaagcgggctgctgctctctgcaggttctgcagcggtctctgtctagtgggtgttttctttttcttagccctgcccctggattgtcagacggcgggcgtctgcctctgaagttagccgtgatttcctctagagccgggtcttatctctggctgcacgttgcctgtgggtgactaatcacacaataacattgtttagggctggaatgaagtcagagctgtttacccccactctataggggttcaatataaaaaggcggcggagaactgtccgagtcagaagcgttcctgcaccggcgctgagagcctgacccggtctgctccgctgtccttgcgcgctgcctcccggctgcccgcgacgctttcgccccagtggaagggccacttgctgcggccgc

SEQ ID NO: 19 - DNA, 35207 bp, Artificial Sequence
  VB-111 entire construct
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtacgtacgtgtacttctgatcggcgatactagggagataaggatgtgcctgacaaaaccacattgttgttgttatcattattatttagttttccttccttgctaactcctgacggaatctttctcacctcaaatgcgaagtactttagtttagaaaagacttggtggaaggggtggtggtggaaaagtagggtgatcttccaaactaatctggttccccgcccgccccagtagctgggattcaagagcgaagagtggggatcgtccccttgtttgatcagaaagacataaaaggaaaatcaagtgaacaatgatcagccccacctccaccccacccccctgcgcgcgcacaatacaatctatttaattgtacttcatacttttcattccaatggggtgactttgcttctggagaaactcttgattcttgaactctggggctggcagctagcctccagaagcaaagtcaccccattggaatgaaaagtatgaagtacaatgaaaagtatgaagtactggctccagaagcaaagtcaccctccagaagcaaagtcaccccattggaatgaaaagtatgaagtacgctagcaaaaggggaagcgggctgctgctctctgcaggttctgcagcggtctctgtctagtgggtgttttctttttcttagccctgcccctggattgtcagacggcgggcgtctgcctctgaagttagccgtgatttcctctagagccgggtcttatctctggctgcacgttgcctgtgggtgactaatcacacaataacattgtttagggctggaatgaagtcagagctgtttacccccactctataggggttcaatataaaaaggcggcggagaactgtccgagtcagaagcgttcctgcaccggcgctgagagcctgacccggtctgctccgctgtccttgcgcgctgcctcccggctgcccgcgacgctttcgccccagtggaagggccacttgctgcggccgctaattctgcagatcgggatccggcatgggcctctccaccgtgcctgacctgctgctgccgctggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccaagcttaggatccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctagctcgagcatgcatctaggcggccgcatggcagaaattcgcgaattcgctagcgttaacggatcctctagacgagatccgaacttgtttattgcagcttataatggttacaaataaagcaatagcatcacaaatttcacaaataaagcatttttttcactgcattctagttgtggtttgtccaaactcatcaatgtatcttatcatgtctagatctgtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagatgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaatttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgctttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaacgcaatagatgccatgaaccacccaactttccccgcgcccgctatgcttccactgcaacaagttgttgccggcggctttgtcccagccaatcagcctcgcccaccttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 20 - DNA, 19 bp, Artificial Sequence
  cis-regulatory element
ggtgactttgcttctggag

SEQ ID NO: 21 - DNA, 19 bp, Artificial Sequence
  cis-regulatory element
ctccagaagcaaagtcacc

SEQ ID NO: 22 - DNA, 19 bp, Artificial Sequence
  cis-regulatory element
gtacttcatacttttcatt

SEQ ID NO: 23 - DNA, 19 bp, Artificial Sequence
  cis-regulatory element
aatgaaaagtatgaagtac

SEQ ID NO: 24 - DNA, 6 bp, Artificial Sequence
  Hypoxia Response element
gcacgt

SEQ ID NO: 25 - protein, 40 aa, Artificial Sequence
  peptide linker
GGGSGGGSGGGSGGGSGGGSGGGSGGGSGGGSGGGSGGGS

(19) United States
(12) Patent Application Publication
(10) Pub. No.: US 2022/0185891 A1
(43) Pub. Date: Jun. 16, 2022

(54) METHODS OF ANTI-TUMOR THERAPY

(71) Applicant: VASCULAR BIOGENICS LTD., Modi'in (IL)

(72) Inventors: Tamar RACHMILEWITZ MINEI, Modi'in (IL); Itzhak MENDEL, Modi'in (IL); Niva YACOV, Modi'in (IL); Eyal BREITBART, Modi'in (IL)

(73) Assignee: VASCULAR BIOGENICS LTD., Modi'in (IL)

(21) Appl. No.: 17/602,963

(22) PCT Filed: Apr. 13, 2020
(86) PCT No.: PCT/IB2020/053477

§ 371 Date: Oct. 11, 2021

Related U.S. Application Data
(60) Provisional application No. 62/833,402, filed on Apr. 12, 2019.

(51) Int. Cl. C07K 16/28; A61K 38/19; A61K 45/06; A61P 35/00

(52) U.S. Cl. CPC C07K 16/2818; A61K 38/191; A61K 2039/505; A61P 35/00; C07K 16/2827; A61K 45/06

(57) ABSTRACT

The disclosure provides methods of treating a tumor in a subject in need thereof comprising administering to the subject the combination of an effective dose of a vector comprising a Fas-chimera gene operably linked to an endothelial cell-specific promoter and an effective dose of an immune checkpoint inhibitor. In some aspects of the disclosure, the immune checkpoint inhibitor is a PD-1 antagonist or PD-L1 antagonist.

### REFERENCE TO RELATED APPLICATION

This application claims the benefit of U.S. Provisional Patent Application No. 62/833,402, filed on Apr. 12, 2019, incorporated herein by reference in its entirety.

### REFERENCE TO SEQUENCE LISTING SUBMITTED ELECTRONICALLY

The content of the electronically submitted sequence listing in ASCII text file (Name: 3182_091PC01_Seqlisting_ST25; Size: 72,084 bytes; and Date of Creation: Mar. 24, 2020) filed with the application is incorporated herein by reference in its entirety

### BACKGROUND OF THE DISCLOSURE

Angiogenesis is a common and major feature of several pathologies. Among these are diseases in which the angiogenesis can improve the disease condition (such as ischemic heart disease) and diseases in which the excessive angiogenesis is a part of the pathology and thus should be eliminated. These latter diseases include diabetes (diabetic retinopathy), cardiovascular diseases (atherosclerosis), chronic inflammation (rheumatoid arthritis), and cancer. Angiogenesis occurs in tumors and permits their growth, invasion and metastasis. In 1971, Folkman proposed that tumor growth and metastases are angiogenesis dependent, and thus inhibiting angiogenesis can be a strategy to arrest tumor growth.

There are several molecules involved in angiogenesis, from transcription factors to growth factors. Hypoxia is an important environmental factor that leads to neovascularization, and it induces release of several cytokines that are pro-angiogenic factors. Among them are vascular endothelial growth factors (VEGF) and their receptors, members of the angiopoietin family, basic fibroblast growth factor, and endothelin-1 (ET-1). These factors are involved in induction of angiogenesis through activation, proliferation and migration of endothelial cells.

Recombinant forms of endogenous inhibitors of angiogenesis were tested for the treatment of cancer. The potential pharmacokinetic, biotechnological and economic drawbacks of chronic delivery of these recombinant inhibitors have led scientists to develop other approaches.

The development of the anti-VEGF monoclonal antibody bevacizumab has validated an antiangiogenic approach as a complementary therapeutic modality to chemotherapy. Several small molecule inhibitors, including second-generation multi-targeted tyrosine kinase inhibitors, have also shown promise as antiangiogenic agents for cancer.

Immune checkpoints also play a role in tumor growth and development. For example, by naturally stimulating immune checkpoints through receptor/ligand interaction, tumor cells are able to evade the host immune system. Thus, molecules blocking immune checkpoints (e.g., immune checkpoint inhibitors) were tested for treatment of cancer. However, these inhibitors worked in only a small percentage of patients with only a few types of tumors. Further, patient response to immune checkpoint therapy is often followed by relapse and disease progression.

The potential pharmacokinetic and economic drawbacks of chronic delivery of recombinant inhibitors, antibodies, and small molecules, as well as the limited activity manifested when applied as monotherapy have led scientists to evaluate gene therapy. However, there are also obstacles limiting successful gene therapy, including duration of expression, induction of the immune response, cytotoxicity of the vectors and tissue specificity. Two general strategies for the cancer gene therapy were proposed: tumor directed or systemic gene therapy. The lack of success in targeting gene therapy products to cancerous cells or their environment by systemic treatments caused most therapies to be administered to the tumor itself.

### BRIEF SUMMARY OF THE DISCLOSURE

The present disclosure also provides a method of reducing or inhibiting the size of a tumor or eliminating a tumor in a subject in need thereof comprising (a) administering to the subject an effective dose of a vector comprising a Fas-chimera gene operably linked to an endothelial cell-specific promoter and (b) administering to the subject an effective dose of an immune checkpoint inhibitor. The disclosure also provides a method of treating a tumor or a metastasis thereof in a subject in need thereof, the method comprising (a) administering to the subject an effective dose of a vector comprising a Fas-chimera gene operably linked to an endothelial cell-specific promoter and (b) administering to the subject an effective dose of an immune checkpoint inhibitor. The disclosure also provides a method of inducing or improving T cell activation in a subject having a tumor comprising (a) administering to the subject an effective dose of a vector comprising a Fas-chimera gene operably linked to an endothelial cell-specific promoter and (b) administering to the subject an effective dose of an immune checkpoint inhibitor. The disclosure also provides a method of inducing or improving the efficacy of an immune checkpoint inhibitor in a subject having a tumor, comprising administering to the subject an effective dose of a vector comprising a Fas-chimera gene operably linked to an endothelial cell-specific promoter and (b) administering to the subject an effective dose of an immune checkpoint inhibitor. The disclosure also provides a method of converting a cold tumor to a hot tumor in a subject in need thereof, comprising (a) administering to the subject an effective dose of a vector comprising a Fas-chimera gene operably linked to an endothelial cell-specific promoter and (b) administering to the subject an effective dose of an immune checkpoint inhibitor.

In some aspects, tumor is derived from or associated with Leukemia, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, non-small cell lung cancer, primary brain tumors (including glioblastima multiforme), gastrointestinal (GI) cancers (including but not limited to cancers of the esophagus, gallbladder, biliary tract, liver, pancreas, stomach, small intestine, large intestine, colon, rectum, and anus), malignant pancreatic insulanoma, malignant carcinoid, urinary bladder cancer, premalignant skin lesions, testicular cancer, lymphomas, thyroid cancer, papillary thyroid cancer, neuroblastoma, neuroendocrine cancer, genitourinary tract cancer, malignant hypercalcemia, cervical cancer, endometrial cancer, adrenal cortical cancer, prostate cancer, Müllerian cancer, ovarian cancer, peritoneal cancer, fallopian tube cancer, or uterine papillary serous carcinoma.

In some aspects, the Fas-chimera gene encodes a polypeptide comprising an extracellular domain of a TNF Receptor 1 (TNFR1) polypeptide fused to a trans-membrane domain and an intracellular domain of a Fas polypeptide. In some aspects, the extracellular domain of the TNFR1 comprises an amino acid sequence at least about 70%, 80%, 90%, 95%, 96%, 97%, 98%, 99%, or 100% identical to SEQ ID NO: 4, wherein the extracellular domain of the TNFR1 is capable of binding to TNF-α. In some aspects, the trans-membrane domain and the intracellular domain of the Fas polypeptide comprises an amino acid sequence at least about 70%, 80%, 90%, 95%, 96%, 97%, 98%, 99%, or 100% identical to SEQ ID NO: 8, wherein the trans-membrane domain and the intracellular domain of the Fas polypeptide is capable of inducing Fas mediated apoptosis. In some aspects, the Fas-chimera gene comprises a first nucleotide sequence, which is at least 70%, 80%, 90%, 95%, 96%, 97%, 98%, 99%, or 100% identical to SEQ ID NO: 3, and a second nucleotide sequence, which is at least about 70%, 80%, 90%, 95%, 96%, 97%, 98%, 99%, or 100% identical to SEQ ID NO: 7.

In some aspects of the disclosure, the endothelial cell-specific promoter comprises a PPE-1 promoter. In some aspects, the endothelial cell-specific promoter further comprises a cis-acting regulatory element. In some aspects, the cis-acting regulatory element comprises a nucleotide sequence at least about 70%, 80%, 90%, 95%, 96%, 97%, 98%, 99%, or 100% identical to SEQ ID NO: 15 or SEQ ID NO: 16. In particular aspects of the disclosure, the cis-acting regulatory element comprises SEQ ID NO: 11 or SEQ ID NO: 12. In some aspects, the cis-acting regulatory element further comprises SEQ ID NO: 13 or SEQ ID NO: 14.

In some aspects of the disclosure, the endothelial cell-specific promoter is a PPE-1-3X promoter. In some aspects, the PPE-1-3X promoter comprises a nucleotide sequence at least about 70%, 80%, 90%, 95%, 96%, 97%, 98%, 99%, or 100% identical to SEQ ID NO: 18, wherein the PPE-1-3X promoter is capable of directing the Fas-chimera gene expression in endothelial cells.

In some aspects, the effective dose of the vector is administered in an amount of about 1×10¹⁰ to about 1×10¹⁶, about 1×10¹¹ to about 1×10¹⁵, about 1×10¹¹ to about 1×10¹⁶, about 1×10¹² to about 1×10¹⁵, about 1×10¹² to about 1×10¹⁶, about 1×10¹² to about 1×10¹⁴, about 5×10¹² to about 1×10¹⁶, about 5×10¹² to about 1×10¹⁵, about 5×10¹² to about 1×10¹⁴, about 1×10¹² to about 1×10¹³, or about 1×10¹³ to about 1×10¹⁴ virus particles. In some aspects, the effective dose of the vector is administered in an amount of about 1×10¹⁶, 1×10¹⁵ 1×10¹⁴ 5×10¹³ 4×10¹³ 3×10¹³ 2×10¹³ 1×10¹³, 9×10¹², 8×10¹², 7×10¹², 6×10¹², 5×10¹², 4×10¹², 3×10¹², 2×10¹², 1×10¹², 9×10¹¹, 8×10¹¹, 7×10¹¹, 6×10¹¹, 5×10¹¹, 4×10¹¹, 3×10¹¹, 2×10¹¹, 1×10¹¹, 9×10¹⁰, 8×10¹⁰, 7×10¹⁰, 6×10¹⁰, 5×10¹⁰, 4×10¹⁰, 3×10¹⁰, 2×10¹⁰, or 1×10¹⁰ virus particles.

In some aspects of the disclosure, the vector and the immune checkpoint inhibitor are administered sequentially. In some aspects, the vector is administered prior to the immune checkpoint inhibitor. In a particular aspect, the vector is administered prior to the immune checkpoint inhibitor and the immune checkpoint is administered upon tumor progression. In other aspects, the immune checkpoint inhibitor is administered prior to the vector.

In some aspects, the vector is repeatedly administered. In some aspects, the vector is repeatedly administered every day, once in about 2 days, once in about 3 days, once in about 4 days, once in about 5 days, once in about 6 days, once in about 7 days, once in about 2 weeks, once in about 3 weeks, once in about 4 weeks, once in about 5 weeks, once in about 6 weeks, once in about 7 weeks, once in about 2 months, or once in about 6 months.

In some aspects of the disclosure, the immune checkpoint inhibitor is repeatedly administered. In some aspects, the immune checkpoint inhibitor is repeatedly administered once in about 7 days, once in about 2 weeks, once in about 3 weeks, once in about 4 weeks, once in about 2 months, once in about 3 months, once in about 4 months, once in about 5 months, or once in about 6 months

In some aspects of the disclosure, the immune checkpoint inhibitor is a PD-1 antagonist. In some aspects, the PD-1 antagonist is administered at an effective amount of less than about 15 mg/kg, less than about 14 mg/kg, less than about 13 mg/kg, less than about 12 mg/kg, less than about 11 mg/kg, less than about 10 mg/kg, less than about 9 mg/kg, less than about 8 mg/kg, less than about 7 mg/kg, less than about 6 mg/kg, less than about 5 mg/kg, less than about 4 mg/kg, less than about 3 mg/kg, less than about 2 mg/kg, or less than about 1 mg/kg. In other aspects, the PD-1 antagonist is administered at an effective amount of a flat dose between about 100 mg to about 600 mg, about 120 mg to about 500 mg, about 140 mg to about 460 mg, about 180 mg to about 420 mg, about 200 mg to about 380 mg, about 220 mg, to about 340 mg, about 230 mg to about 300 mg, or about 230 mg to about 260 mg. In some aspects, the PD-1 antagonist is administered at an effective amount of a flat dose between about 400 mg to about 600 mg, about 450 mg to about 520 mg, about 460 mg to about 510 mg, or about 470 mg to about 500 mg. In some aspects, the PD-1 antagonist is administered at an effective amount of a flat dose of about 60 mg, about 80 mg, about 100 mg, about 120 mg, about 140 mg, about 160 mg, about 180 mg, about 200 mg, about 220 mg, about 240 mg, about 260 mg, about 280 mg, about 300 mg, about 320 mg, about 340 mg, about 360 mg, about 380 mg, about 400 mg, about 420 mg, about 440 mg, about 460 mg, about 480 mg, about 500 mg, about 520 mg, about 540 mg, about 560 mg, about 580 mg, or about 600 mg.

In particular aspects, the PD-1 antagonist is an antibody that binds to PD-1. In some aspects, the antibody is a monoclonal antibody, a humanized antibody, a human antibody, a single chain antibody, or a chimeric antibody. In more particular aspects, the antibody is selected from the group consisting of nivolumab, pembrolizumab, camrelizumab, cemiplimab, sintilimab, and PDR001. In a particular aspect, the PD-1 antagonist is nivolumab.

In some aspects of the disclosure, the vector is administered at an effective amount of 3×10¹² to 3×10¹³ virus particles and the nivolumab is administered at an effective amount of 2 mg/kg to 12 mg/kg. In other aspects, the vector is administered at an effective amount of 3×10¹² to 3×10¹³ virus particles and the nivolumab is administered at a flat dose of 460 mg to 500 mg.

In some aspects, the vector is administered every 2 months and the nivolumab is administered every 2 weeks. In other aspects, the vector is administered every 2 months and the nivolumab is administered every two months. In some aspects, the nivolumab is administered one month after each administration of the vector

In some aspects of the disclosure, the PD-1 antagonist is an antibody that binds to PD-L1. In some aspects, the antibody is a monoclonal antibody, a humanized antibody, a human antibody, a single chain antibody, or a chimeric antibody. In particular aspects, the antibody is selected from the group consisting of atezolizumab, avelumab, durvalumab, and BMS-936559.

Some aspects of the present disclosure comprise further administering to the subject an effective dose of one or more chemotherapeutic agents. In some aspects, the one or more chemotherapeutic agents is selected from the group consisting of Acivicin; Aclarubicin; Acodazole Hydrochloride; Acronine; Adriamycin; Adozelesin; Aldesleukin; Alimta; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bevacizumab, Bizelesin; Bleomycin Sulfate; Brequinar Sodium; Bropirimine; Busulfan; Cactinomycin; Calusterone; Caracemide; Carbetimer; Carboplatin; Carmustine (BiCNU); Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Gliadel® wafer; Hydroxyurea; Idarubicin Hydrochloride; Ifosfamide; Ilmofosine; Interferon Alfa-2a; Interferon Alfa-2b; Interferon Alfa-nl; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine (CCNU); Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; pazotinib; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sorafinib; Sparfosate Sodium; Sparsomycin; Spirogermanium Hydrochloride; Spiromustine; Spiroplatin; Streptonigrin; Streptozocin; Sulofenur; Sunitinib; Talisomycin; Taxol; Tecogalan Sodium; Tegafur; Teloxantrone Hydrochloride; Temoporfin; Temozolomide; Teniposide; Teroxirone; Testolactone; Thiamiprine; Thioguanine; Thiotepa; Tiazofuirin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; and Zorubicin Hydrochloride.

In particular aspects of the disclosure, the vector comprises, consists of, or consists essentially of SEQ ID NO: 19. In some aspects, the vector is an isolated virus having European Collection of Cell Cultures (ECACC) Accession Number 13021201.

### BRIEF DESCRIPTION OF THE DRAWINGS/FIGURES

FIG. 1 shows a study design for combination therapy of an anti-PD-L1 antibody and an Ad5-PPE-1-3X-Fas-c vector, VB-111. 3 days acclimation: mice were allowed to acclimate in their cages for three days prior to disease induction; Disease Induction: the mouse footpad was inoculated with D122 cells (metastatic lung tumor model) and monitored for tumor growth until tumors reached 7 mm³; Amputation Day 0: when tumors reached the target size, the tumors were removed by amputation, starting “Day 0”; VB-111 I.V.: 5 days after tumor amputation, mice were treated with intravenous injection of VB-111 vector; Anti-PD-L1 antibody: some groups of mice were also given anti-PD-L1 antibody intraperitoneally at days 5, 8, and 11 while VB-111 is given intravenously at day 5.

FIG. 2 shows mouse lung weight (in grams) following treatment with saline (control) VB-111 alone (1×10¹¹ or 1×10⁹ viral particles), an anti-PD-L1 antibody alone (200 μg). or VB-111 (1×10¹¹ viral particles) combined with the anti-PD-L1 antibody (200 μg).

FIG. 3 shows mouse lung tumor burden (in grams) following treatment with saline (control) VB-111 alone (1×10¹¹ or 1×10⁹ viral particles), an anti-PD-L1 antibody alone (200 μg), or VB-111 (1×10¹¹ viral particles) combined with the anti-PD-L1 antibody (200 μg).

FIG. 4 shows mouse melanoma tumor volume (in mm³) following treatment with saline (squares), VB-111 alone at 1×10¹¹ viral particles (circles), an anti-PD-L1 antibody alone at 200 μg (triangles), or VB-111 at 1×10¹¹ viral particles combined with the anti-PD-L1 antibody at 200 μg (stars). Arrows indicate treatment days 9, 12, and 14. I.V., intravenous; I.P., intraperitoneal.

FIG. 5 shows the study design for the first segment of a phase I/II clinical trial of trial of VB-111 therapy combined with an anti-PD-1 antibody, e.g., nivolumab. In this design, subjects will be administered VB-111 at 3×10¹² viral particles or 1×10¹³ viral particles combined with nivolumab at 3 mg/kg. DLT: dose limiting toxicity.

FIG. 6 shows the study design for the second segment of a phase I/II clinical trial of VB-111 therapy combined with an anti-PD-1 antibody, e.g., nivolumab. In the second segment, subjects will be administered VB-111 at 1×10¹³ viral particles combined with nivolumab at 3 mg/kg (Arm 1) or nivolumab at 3 mg/kg (Arm 2). DLT: dose limiting toxicity.

FIG. 7 shows the study design for open label, single-arm phase II study of VB-111 in combination with anti-PD1 antibody, nivolumab, in patients with advanced, refractory Metastatic Colorectal Cancer. Patients will undergo pre-treatment biopsy and one post-treatment biopsy at Day 1 of Cycle 2 or Day 1 of Cycle 4.

### EMBODIMENTS

1. A method of reducing the size or inhibiting the growth of a tumor or eliminating a tumor in a subject in need thereof comprising (a) administering to the subject an effective dose of a vector comprising a Fas-chimera gene operably linked to an endothelial cell-specific promoter and (b) administering to the subject an effective dose of an immune checkpoint inhibitor.

2. A method of treating a tumor or a metastasis thereof in a subject in need thereof, the method comprising (a) administering to the subject an effective dose of a vector comprising a Fas-chimera gene operably linked to an endothelial cell-specific promoter and (b) administering to the subject an effective dose of an immune checkpoint inhibitor.

3. A method of inducing or improving T cell activation in a subject having a tumor comprising (a) administering to the subject an effective dose of a vector comprising a Fas-chimera gene operably linked to an endothelial cell-specific promoter and (b) administering to the subject an effective dose of an immune checkpoint inhibitor.

4. A method of inducing or improving the efficacy of an immune checkpoint inhibitor in a subject having a tumor, comprising administering to the subject an effective dose of a vector comprising a Fas-chimera gene operably linked to an endothelial cell-specific promoter and (b) administering to the subject an effective dose of an immune checkpoint inhibitor.

5. A method of converting a cold tumor to a hot tumor in a subject in need thereof, comprising (a) administering to the subject an effective dose of a vector comprising a Fas-chimera gene operably linked to an endothelial cell-specific promoter and (b) administering to the subject an effective dose of an immune checkpoint inhibitor.

6. The method of any one of embodiments 1-5, wherein the tumor is derived from or associated with Leukemia, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, non-small cell lung cancer, primary brain tumors (including glioblastoma multiforme), gastrointestinal (GI) cancers (including but not limited to cancers of the esophagus, gallbladder, biliary tract, liver, pancreas, stomach, small intestine, large intestine, colon, rectum, and anus), malignant pancreatic insulanoma, malignant carcinoid, urinary bladder cancer, premalignant skin lesions, testicular cancer, lymphomas, thyroid cancer, papillary thyroid cancer, neuroblastoma, glioblastima multiforme, neuroendocrine cancer, genitourinary tract cancer, malignant hypercalcemia, cervical cancer, endometrial cancer, adrenal cortical cancer, prostate cancer, Mullerian cancer, ovarian cancer, peritoneal cancer, fallopian tube cancer, or uterine papillary serous carcinoma.

7. The method of any one of embodiments 1-6, wherein the Fas-chimera gene encodes a polypeptide comprising an extracellular domain of a TNF Receptor 1 (TNFR1) polypeptide fused to a trans-membrane domain and an intracellular domain of a Fas polypeptide.

8. The method of embodiment 7, wherein the extracellular domain of the TNFR1 comprises an amino acid sequence at least about 70%, 80%, 90%, 95%, 96%, 97%, 98%, 99%, or 100% identical to SEQ ID NO: 4, wherein the extracellular domain of the TNFR1 is capable of binding to TNF-α.

9. The method of embodiment 8, wherein the trans-membrane domain and the intracellular domain of the Fas polypeptide comprises an amino acid sequence at least about 70%, 80%, 90%, 95%, 96%, 97%, 98%, 99%, or 100% identical to SEQ ID NO: 8, wherein the trans-membrane domain and the intracellular domain of the Fas polypeptide is capable of inducing Fas mediated apoptosis.

10. The method of any of embodiments 1-9, wherein the Fas-chimera gene comprises a first nucleotide sequence, which is at least 70%, 80%, 90%, 95%, 96%, 97%, 98%, 99%, or 100% identical to SEQ ID NO: 3, and a second nucleotide sequence, which is at least about 70%, 80%, 90%, 95%, 96%, 97%, 98%, 99%, or 100% identical to SEQ ID NO: 7.

11. The method of any one of embodiments 1-10, wherein the endothelial cell-specific promoter comprises a PPE-1 promoter.

12. The method of any one of embodiments 1-11, wherein the endothelial cell-specific promoter further comprises a cis-acting regulatory element.

13. The method of embodiment 12, wherein the cis-acting regulatory element comprises a nucleotide sequence at least about 70%, 80%, 90%, 95%, 96%, 97%, 98%, 99%, or 100% identical to SEQ ID NO: 15 or SEQ ID NO: 16.

14. The method of embodiment 13, wherein the cis-acting regulatory element comprises SEQ ID NO: 11 or SEQ ID NO: 12.

15. The method of embodiment 14, wherein the cis-acting regulatory element further comprises SEQ ID NO: 13 or SEQ ID NO: 14.

16. The method of any one of embodiments 1-15, wherein the endothelial cell-specific promoter is a PPE-1-3X promoter.

17. The method of embodiment 16, wherein the PPE-1-3X promoter comprises a nucleotide sequence at least about 70%, 80%, 90%, 95%, 96%, 97%, 98%, 99%, or 100% identical to SEQ ID NO: 18, wherein the PPE-1-3X promoter is capable of directing the Fas-chimera gene expression in endothelial cells.

18. The method of any one of embodiments 1-17, wherein the effective dose of the vector is administered in an amount of about 1×10¹⁰ to about 1×10¹⁶, about 1×10¹¹ to about 1×10¹⁵, about 1×10¹¹ to about 1×10¹⁶, about 1×10¹² to about 1×10¹⁵, about 1×10¹² to about 1×10¹⁶, about 1×10¹² to about 1×10¹⁴, about 5×10¹² to about 1×10¹⁶, about 5×10¹² to about 1×10¹⁵, about 5×10¹² to about 1×10¹⁴, about 1×10¹² to about 1×10¹³, or about 1×10¹³ to about 1×10¹⁴ virus particles.

19. The method of any one of embodiments 1-18, wherein the effective dose of the vector is administered in an amount of about 1×10¹⁶, 1×10¹⁵, 1×10¹⁴, 5×10¹³, 4×10¹³, 3×10¹³, 2×10¹³, 1×10¹³, 9×10¹², 8×10¹², 7×10¹², 6×10¹², 5×10¹², 4×10¹², 3×10¹², 2×10¹², 1×10¹², 9×10¹¹, 8×10¹¹, 7×10¹¹, 6×10¹¹, 5×10¹¹, 4×10¹¹, 3×10¹¹, 2×10¹¹, 1×10¹¹, 9×10¹⁰, 8×10¹⁰, 7×10¹⁰, 6×10¹⁰, 5×10¹⁰, 4×10¹⁰, 3×10¹⁰, 2×10¹⁰, or 1×10¹⁰ virus particles.

20. The method of any one of embodiments 1-19, wherein the vector and the immune checkpoint inhibitor are administered sequentially.

21. The method of embodiment 20, wherein the vector is administered prior to the immune checkpoint inhibitor.

22. The method of embodiment 21, wherein the immune checkpoint is administered upon tumor progression.

23. The method of embodiment 20, wherein the immune checkpoint inhibitor is administered prior to the vector.

24. The method of any one of embodiments 1-23, wherein the vector is repeatedly administered.

25. The method of embodiment 24, wherein the vector is repeatedly administered every day, once in about 2 days, once in about 3 days, once in about 4 days, once in about 5 days, once in about 6 days, once in about 7 days, once in about 2 weeks, once in about 3 weeks, once in about 4 weeks, once in about 5 weeks, once in about 6 weeks, once in about 7 weeks, once in about 2 months, or once in about 6 months.

26. The method of any one of embodiments 1 to 25, wherein the immune checkpoint inhibitor is repeatedly administered.

27. The method of embodiment 26, wherein the immune checkpoint inhibitor is repeatedly administered once in about 7 days, once in about 2 weeks, once in about 3 weeks, once in about 4 weeks, once in about 2 months, once in about 3 months, once in about 4 months, once in about 5 months, or once in about 6 months.

28. The method of any one of embodiments 1-27, wherein the immune checkpoint inhibitor is a PD-1 antagonist.

29. The method of embodiment 28, wherein the PD-1 antagonist is administered at an effective amount of less than about 15 mg/kg, less than about 14 mg/kg, less than about 13 mg/kg, less than about 12 mg/kg, less than about 11 mg/kg, less than about 10 mg/kg, less than about 9 mg/kg, less than about 8 mg/kg, less than about 7 mg/kg, less than about 6 mg/kg, less than about 5 mg/kg, less than about 4 mg/kg, less than about 3 mg/kg, less than about 2 mg/kg, or less than about 1 mg/kg.

30. The method of embodiment 28, wherein the PD-1 antagonist is administered at an effective amount of a flat dose between about 100 mg to about 600 mg, about 120 mg to about 500 mg, about 140 mg to about 460 mg, about 180 mg to about 420 mg, about 200 mg to about 380 mg, about 220 mg, to about 340 mg, about 230 mg to about 300 mg, or about 230 mg to about 260 mg.

31. The method of embodiment 28, wherein the PD-1 antagonist is administered at an effective amount of a flat dose between about 400 mg to about 600 mg, about 450 mg to about 520 mg, about 460 mg to about 510 mg, or about 470 mg to about 500 mg.

32. The method of embodiment 28, wherein the PD-1 antagonist is administered at an effective amount of a flat dose of about 60 mg, about 80 mg, about 100 mg, about 120 mg, about 140 mg, about 160 mg, about 180 mg, about 200 mg, about 220 mg, about 240 mg, about 260 mg, about 280 mg, about 300 mg, about 320 mg, about 340 mg, about 360 mg, about 380 mg, about 400 mg, about 420 mg, about 440 mg, about 460 mg, about 480 mg, about 500 mg, about 520 mg, about 540 mg, about 560 mg, about 580 mg, or about 600 mg.

33. The method of any one of embodiments 28-32, wherein the PD-1 antagonist is an antibody that binds to PD-1.

34. The method of embodiment 33, wherein the antibody is a monoclonal antibody, a humanized antibody, a human antibody, a single chain antibody, or a chimeric antibody.

35. The method of embodiment 33 or 34, wherein the antibody is selected from the group consisting of nivolumab, pembrolizumab, camrelizumab, cemiplimab, sintilimab, and PDR001.

36. The method of any one of embodiments 28-35, wherein the PD-1 antagonist is nivolumab.

37. The method of any one of embodiments 28-32, wherein the PD-1 antagonist is an antibody that binds to PD-L1.

38. The method of embodiment 37, wherein the antibody is a monoclonal antibody, a humanized antibody, a human antibody, a single chain antibody, or a chimeric antibody.

39. The method of embodiment 37 or 38, wherein the antibody is selected from the group consisting of atezolizumab, avelumab, durvalumab, and BMS-936559.

40. The method of any one of embodiments 1-39, further comprising administering to the subject an effective dose of one or more chemotherapeutic agents.

41. The method of embodiment 40, wherein the one or more chemotherapeutic agents is selected from the group consisting of Acivicin; Aclarubicin; Acodazole Hydrochloride; Acronine; Adriamycin; Adozelesin; Aldesleukin; Alimta; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bevacizumab, Bizelesin; Bleomycin Sulfate; Brequinar Sodium; Bropirimine; Busulfan; Cactinomycin; Calusterone; Caracemide; Carbetimer; Carboplatin; Carmustine (BiCNU); Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Gliadel® wafer; Hydroxyurea; Idarubicin Hydrochloride; Ifosfamide; Ilmofosine; Interferon Alfa-2a; Interferon Alfa-2b; Interferon Alfa-nl; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine (CCNU); Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; pazotinib; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sorafinib; Sparfosate Sodium; Sparsomycin; Spirogermanium Hydrochloride; Spiromustine; Spiroplatin; Streptonigrin; Streptozocin; Sulofenur; Sunitinib; Talisomycin; Taxol; Tecogalan Sodium; Tegafur; Teloxantrone Hydrochloride; Temoporfin; Temozolomide; Teniposide; Teroxirone; Testolactone; Thiamiprine; Thioguanine; Thiotepa; Tiazofuirin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; and Zorubicin Hydrochloride.

42. The method of any one of embodiments 1-41, wherein the vector comprises, consists of, or consists essentially of SEQ ID NO: 19.

43. The method of any one of embodiments 1-42, wherein the vector is an isolated virus having European Collection of Cell Cultures (ECACC) Accession Number 13021201.

### DETAILED DESCRIPTION OF THE DISCLOSURE

### I. Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided.

Throughout this disclosure, the term “a” or “an” entity refers to one or more of that entity; for example, “a polynucleotide,” is understood to represent one or more polynucleotides. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

As used herein, “antibody” means an intact immunoglobulin, an antigen-binding fragment thereof, or an antigen-binding molecule. Antibodies of this disclosure can be of any isotype or class (e.g., M, D, G, E and A) or any subclass (e.g., G1-4, A1-2) and can have either a kappa (κ) or lambda (λ) light chain.

The term “effective amount” as used herein refers to an amount effective, at dosages and for periods of time necessary, to achieve a desired result. A desired result can be, for example, reduction or inhibition of neo-vascularization or angiogenesis in vitro or in vivo; reduction or inhibition of the size of a tumor; or inducing or improving T cell activation. An effective amount need not be a “cure” or complete removal of neo-vascularization or angiogenesis. In some embodiments, an effective amount can reduce a size or volume of a tumor. In some embodiments, an effective amount can reduce or ameliorate one or more symptoms of a cancer.

As used herein, the phrase “treating a tumor” refers to inhibiting the growth of a tumor, reducing the size of a tumor, eliminating a tumor, preventing the recurrence of a tumor, and combinations thereof.

The term “polynucleotide” or “nucleotide” is intended to encompass a singular nucleic acid as well as plural nucleic acids, and refers to an isolated nucleic acid molecule or construct, e.g., messenger RNA (mRNA) or plasmid DNA (pDNA). In certain embodiments, a polynucleotide comprises a conventional phosphodiester bond or a non-conventional bond (e.g., an amide bond, such as found in peptide nucleic acids (PNA)).

As used herein, a “polynucleotide,” “nucleotide,” or “nucleic acid” can be used interchangeably and contain the nucleotide sequence of the full-length cDNA sequence, including the untranslated 5′ and 3′ sequences, the coding sequences, as well as fragments, epitopes, domains, and variants of the nucleic acid sequence. The polynucleotide can be composed of any polyribonucleotide or polydeoxyribonucleotide, which can be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, the polynucleotides can be composed of triple-stranded regions comprising RNA or DNA or both RNA and DNA. Polynucleotides can also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

In the present disclosure, a polypeptide can be composed of amino acids joined to each other by peptide bonds or modified peptide bonds, i.e., peptide isosteres, and can contain amino acids other than the 20 gene-encoded amino acids (e.g. non-naturally occurring amino acids). The polypeptides of the present disclosure can be modified by either natural process, such as posttranslational processing, or by chemical modification techniques which are well known in the art. Such modifications are well described in basic texts and in more detailed monographs, as well as in a voluminous research literature. Modifications can occur anywhere in the polypeptide, including the peptide backbone, the amino acid side-chains and the amino or carboxyl termini. It will be appreciated that the same type of modification can be present in the same or varying degrees at several sites in a given polypeptide. Also, a given polypeptide can contain many types of modifications. Polypeptides can be branched, for example, as a result of ubiquitination, and they can be cyclic, with or without branching. Cyclic, branched, and branched cyclic polypeptides can result from post-translation natural processes or can be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins—Structure And Molecular Properties, 2nd Ed., T.E. Creighton, W.H. Freeman and Company, New York (1993); Posttranslational Covalent Modification of Proteins, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992).)

The terms “fragment,” “variant,” “derivative” and “analog” when referring to any polypeptide or polynucleotide of the present disclosure include any polypeptides or polynucleotides which retain at least some activities, i.e., the ability to function as any naturally-occurring function of the polypeptide or polynucleotide. For example, a “fragment,” “variant,” “derivative” and “analog” of Tumor necrosis factor Receptor 1 (TNFR1) has some activities of the naturally occurring full-length TNFR1, e.g., the ability to bind to TNFR1 ligand, i.e., TNF-alpha or lymphotoxin. In another example, a “fragment,” “variant,” “derivative” and “analog” of a Fas polypeptide have some activities of a naturally-occurring full-length Fas polypeptide, e.g., the ability to induce apoptosis. In other examples, a “fragment,” “variant,” “derivative” and “analog” of an endothelial cell-specific promoter can induce endothelial cell-specific expression of a gene operably linked to the promoter. Additional non-limiting examples of the various fragments, variants, analogues, or derivatives of the TNFR1, Fas polypeptide, and endothelial cell-specific promoters are described below.

The term “percent sequence identity” between two polynucleotide or polypeptide sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence.

The percentage of sequence identity is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences can be accomplished using readily available software both for online use and for download. Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

One skilled in the art will appreciate that the generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2, available from www.clustal.org. Another suitable program is MUSCLE, available from www.drive5.com/muscle/. ClustalW2 and MUSCLE are alternatively available, e.g., from the EBI.

It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

As used herein, the terms “linked,” “fused,” “fusion,” “chimeric,” and “chimera” are used interchangeably. These terms refer to the joining together of two more elements or components, by whatever means including chemical conjugation or recombinant means. An “in-frame fusion” refers to the joining of two or more open reading frames (ORFs) to form a continuous longer ORF, in a manner that maintains the correct reading frame of the original ORFs. Thus, the resulting recombinant fusion or chimeric protein is a single protein containing two or more segments that correspond to polypeptides encoded by the original ORFs (which segments are not normally so joined in nature.) Although the reading frame is thus made continuous throughout the fused segments, the segments can be physically or spatially separated by, for example, in-frame linker sequence.

The term “heterologous nucleotide sequence” means that a polynucleotide is derived from a distinct entity from that of the entity to which it is being compared. For instance, a heterologous polynucleotide can be synthetic, or derived from a different species, different cell type of an individual, or the same or different type of cell of distinct individuals. In one aspect, a heterologous nucleotide sequence can be a polynucleotide operably linked to another polynucleotide to produce a fusion polynucleotide. In some aspects, a heterologous nucleotide sequence can encode a polypeptide. For example, a heterologous nucleotide sequence can be a promoter element operably linked to a gene encoding a polypetide. A heterologous nucleotide sequence can also include other cis-regulatory elements operably linked to a gene encoding a polypeptide. In other aspects, a heterologous nucleotide sequence does not encode a polypeptide.

The term “expression” as used herein refers to a process by which a gene produces a biochemical, for example, an RNA or polypeptide. The process includes any manifestation of the functional presence of the gene within the cell including, without limitation, gene knockdown as well as both transient expression and stable expression. It includes without limitation transcription of the gene into messenger RNA (mRNA), transfer RNA (tRNA), small hairpin RNA (shRNA), small interfering RNA (siRNA) or any other RNA product and the translation of such mRNA into polypeptide(s). If the final desired product is biochemical, expression includes the creation of that biochemical and any precursors.

The term “complementarity determining region” (CDR) as used herein refers to the amino acid residues of an antibody which are responsible for binding to an antigen. The CDR regions of an antibody are found within the hypervariable region of both heavy and light chains of the antibody. Full length antibodies comprise three CDR regions in the heavy chain variable domain and three CDR regions in the light chain variable domain.

The term “anti-tumor response” as used herein refers to a subject's bodily response against the presence of a tumor. For example, in some aspects the anti-tumor response in the present disclosure can be an anti-tumor immune response. In some aspects, an anti-tumor immune response is characterized by the presence of tumor-infiltrating CD8⁺ lymphocytes within the tumor bed. In some aspects, an anti-tumor immune response is characterized by a particular cytokine profile in the subject. In some aspects, an anti-tumor immune response is characterized by the presence of circulating anti-tumor antibodies in the subject directed against tumor markers or tumor tissue. The term “cold tumor” as used herein refers to a tumor with little or no immune cells present within the tumor. For example, a cold tumor may have little or no tumor infiltrating lymphocytes (e.g., T cells and B cells), Natural Killer (NK) cells, or macrophage cells present within the tumor microenvironment. A tumor need not be completely void of immune cells to be a cold tumor.

The term “hot tumor” as used herein refers to a tumor with increased presence of immune cells within the tumor compared to a cold tumor. For example, a hot tumor may have increased presence of tumor infiltrating lymphocytes (e.g., T cells and B cells), Natural Killer (NK) cells, or macrophage cells within the tumor microenvironment compared to a cold tumor.

The term “immune checkpoint” as used herein refers to biological molecules that serve as positive or negative regulators of the immune system. Immune checkpoints play roles in maintaining self-tolerance, preventing autoimmunity and protecting tissues from immune collateral damage. Immune checkpoint molecules can include, but are not limited to CD27, CD28, CD40, CD122, CD137, OX40, glucocorticoid-induced TNFR family related gene (GITR), inducible T cell costimulator (ICOS), A2AR, B7-H3, B7-H4, BTLA, CTLA-4, IDO, KIR, LAG3, NOX2, PD-1, PD-L1, PD-L2, TIM-3, VISTA, and SIGLEC7.

The term “repeatedly administered” as used herein refers to administration of a therapeutic agent on a repeated basis at defined, fixed intervals. The intervals of time between each administration can be altered during the course of the repeated administration and can be as long as 1 day, 2 days, 3 days, 4 days, 5 days, 6 days, 7 days, 2 weeks, 3 weeks, 4 weeks, 5 weeks, 6 weeks, 7 weeks, 2 months, 3 months, 4 months, 5 months, 6 months, 7 months, 8 months, 9 months, 10 months, 11 months, 1 year, or more.

The term “combination therapy” as used herein refers to the administration of two or more therapeutic modalities to treat a disease or condition. In one aspect of the present disclosure, combination therapy refers to the administration of a vector and an immune checkpoint inhibitor to a subject in need thereof. In some embodiments, the combination therapy comprises administering the checkpoint inhibitor prior to administering the vector. In another embodiment, the combination therapy comprises administering the checkpoint inhibitor concurrently with administration of the vector. In another embodiment, the combination therapy comprises administering the checkpoint inhibitor after administering the vector.

The term “adenovirus” as used herein refers to a human adenovirus of the Adenoviridae family. An adenovirus of the present disclosure can include, for example, an adenovirus from any one of seven species and 57 serotypes, including species A (serotypes 12, 18, and 31), species B (serotypes 3, 7, 11, 14, 16, 21, 34, 35, 50, and 55), species C (serotypes 1, 2, 5, 6, and 57), species D (8, 9, 10, 13, 15, 17, 19, 20, 22-30, 32, 33, 36-39, 42-49, 51, 53, 54, and 56), species E (serotype 4), species F (serotype 40 and 41), or species G (serotype 52). The term “adenovirus vector” as used herein refers to an adenovirus that has been genetically modified to behave differently from the natural wildtype virus. For example, an adenovirus vector may be modified so that it is unable to replicate outside of a particular packaging cell line. In some aspects, an adenovirus vector is genetically modified to carry one or more genes encoding non-adenoviral proteins.

### II. Treatment Methods of the Disclosure

Although a wide variety of chemotherapeutic drugs are available for treating different tumor types, cancer remains among the leading causes of death worldwide. Chemotherapetuic drugs often have undesirable toxicity due to their indiscriminant targeting of rapidly dividing cells, killing both tumor cells and rapidly divding healthy cells. Other agents are limited to treating specific tumor types with specific genetic mutations. These problems are compounded when a primary tumor metastasizes to other regions of the body, making effectice treatment more difficult.

Immune checkpoints play a role in tumor growth and development. By naturally stimulating immune checkpoints through receptor/ligand interaction, tumor cells are able to evade the host immune system. Thus, molecules blocking immune checkpoints (e.g., immune checkpoint inhibitors) were tested for treatment of cancer. However, these inhibitors worked in only a small percentage of patients with only a few types of tumors. Further, patient response to immune checkpoint therapy is often followed by relapse and disease progression.

The present disclosure provides a method of reducing or inhibiting the size of a tumor or eliminating a tumor in a subject in need thereof comprising (a) administering to the subject an effective dose of a vector comprising a Fas-chimera gene operably linked to an endothelial cell-specific promoter and (b) administering to the subject an effective dose of an immune checkpoint inhibitor. In particular aspects, the tumor size in the subject is reduced or inhibited, or the tumor is eliminated compared to a tumor in a subject without administration of the vector.

The present disclosure also provides a method of treating a tumor or a metastasis thereof in a subject in need thereof comprising (a) administering to the subject an effective dose of a vector comprising a Fas-chimera gene operably linked to an endothelial cell-specific promoter and (b) administering to the subject an effective dose of an immune checkpoint inhibitor. In particular aspects, the tumor or metastasis thereof in the subject is treated compared to a tumor in a subject without administration of the vector. By naturally stimulating immune checkpoints, tumor cells can downregulate anti-tumor T cell activity and evade the host's anti-tumor immune response. This results in tumor-induced T cell tolerance, and allows the tumor to continue to grow unchecked by the host immune system. Thus, immune checkpoint inhibitors are studied as cancer treatment agents. However, immune checkpoint inhibitors are not effective therapeutic agents against “cold tumors”—tumors with little or no immune cells present within the tumor. A cold tumor may have little or no tumor infiltrating lymphocytes (e.g., T cells and B cells), Natural Killer (NK) cells, or macrophage cells present within the tumor microenvironment. In contrast a hot tumor is a tumor with increased presence of immune cells within the tumor compared to a cold tumor. For example, a hot tumor may have increased presence of tumor infiltrating lymphocytes (e.g., T cells and B cells), Natural Killer (NK) cells, or macrophage cells within the tumor microenvironment compared to a cold tumor.

The present disclosure also provides a method of inducing or improving T cell activation in a subject having a tumor comprising (a) administering to the subject an effective dose of a vector comprising a Fas-chimera gene operably linked to an endothelial cell-specific promoter and (b) administering to the subject an effective dose of an immune checkpoint inhibitor. In particular aspects, the T cell activation is induced or improved in the subject compared to T cell activation in a subject without administration of the vector.

The present disclosure also provides a method of inducing or improving the efficacy of an immune checkpoint inhibitor comprising (a) administering to the subject an effective dose of a vector comprising a Fas-chimera gene operably linked to an endothelial cell-specific promoter and (b) administering to the subject an effective dose of an immune checkpoint inhibitor. In particular aspects, the efficacy of the immune checkpoint inhibitor is induced or improved in the subject compared to efficacy of the immune checkpoint inhibitor in a subject without administration of the vector.

The present disclosure also provides a method of converting a cold tumor to a hot tumor in a subject in need thereof comprising (a) administering to the subject an effective dose of a vector comprising a Fas-chimera gene operably linked to an endothelial cell-specific promoter and (b) administering to the subject an effective dose of an immune checkpoint inhibitor. In particular aspects, the cold tumor in the subject is converted to a hot tumor compared to a cold tumor in a subject without administration of the vector.

Tumor growth can be measured by techniques known in the art, including but not limited to magnetic resonance imaging (MM) scan, functional MM (fMRI) scan, computerized tomography (CT) scan, or positron emission tomography (PET) scan. In a particular aspect, the growth of the tumor is measured by MRI. In some aspects, the tumor of the subject is a recurrent tumor that arose during treatment with the vector. In yet other embodiments, the tumor of the subject is a metastatic tumor that arose during treatment with the vector.

In some aspects, the methods of the present disclosure increase overall survival of the subject. In some aspects, the methods of the present disclosure increase progression-free survival in the subject.

The term “subject” or “individual” or “animal” or “patient” or “mammal,” is meant any subject, particularly a mammalian subject, having or being expected to have a positive response to the methods of the disclosure. In some aspects, the subject is a human. In some aspects, the subject is a cancer patient.

A. Immune Checkpoint Inhibitors

The methods of the present disclosure comprise administering to a subject an effective dose of a vector comprising a Fas-chimera gene operably linked to an endothelial cell-specific promoter and also administering to the subject an effective dose of an immune checkpoint inhibitor.

Immune checkpoints are biological molecules involved in stimulating or inhibiting an immune response. The immune system naturally attempts to eliminate tumor cells by activating an anti-tumor immune response directed against cells harboring tumor antigens. The anti-tumor immune response may include tumor-specific CD8⁺ lymphocytes (cytotoxic T-cells), natural killer (NK) cells, macrophages, and other immune cells, which migrate to the tumor site, infiltrate the tumor, and kill the tumor cells. Throughout the immune response process, various signaling checkpoints are in place to stimulate or inhibit T-cell activation, thereby regulating the extent and duration of the anti-tumor response.

Some immune checkpoints contribute to stimulating an immune response (e.g., stimulating T-cell activation). Stimulatory immune checkpoints include, but are not limited to, CD27, CD28, CD40, CD4OL (CD154), CD58, CD80, CD86, CD122, CD137 (4-1BB), CD134 (OX40), CD252 (OX4OL), and CD278 (ICOS). Other immune checkpoints exert inhibitory effects on an immune response (e.g., suppressing T-cell activation). Inhibitory immune checkpoints include, but are not limited to adenosine A2A receptor (A2AR), CD152 (CTLA-4), CD272 (BTLA), CD276 (B7-H3), IDO, TDO, killer cell immunoglobulin-like receptor (KIR), lymphocyte activation gene 3 (LAG3), NOX2, VTCN1 (B7-H4), PD-1, PD-L1, PD-L2, T-cell immunoglobulin and mucin domain-3 (TIM3), CD328 (SIGLEC7), CD329 (SIGLEC9), and T-cell immunoreceptor with Ig and ITIM domains (TIGIT).

Tumors can evade a host anti-tumor response by engaging inhibitory immune checkpoints and downregulating the anti-tumor response. Thus, molecules blocking immune checkpoints (e.g., immune checkpoint inhibitors) are studied for treatment of cancer. However, these inhibitors worked in only a small percentage of patients with only a few types of tumors. Further, patient response to immune checkpoint inhibitor therapy is often followed by relapse and disease progression.

In some aspects, the immune checkpoint inhibitor useful in the methods of the present disclosure is a molecule that binds to an immune checkpoint receptor or immune checkpoint receptor ligand. In some aspects, the immune checkpoint inhibitor is an antibody. In some aspects, the antibody is a monoclonal antibody, a humanized antibody, a human antibody, a single chain antibody, or a chimeric antibody. In another aspect, the immune checkpoint inhibitor comprises Fab, F(ab)2, Fv, or scFv.

In some aspects, the immune checkpoint inhibitor binds to an immune checkpoint receptor or immune checkpoint receptor ligand involved in suppressing T-cell activation. In particular aspects, the immune checkpoint inhibitor is a molecule that inhibits T-cell stimulating activity of A2A receptor (A2AR), CD152 (CTLA-4), CD272 (BTLA), CD276 (B7-H3), IDO, TDO, killer cell immunoglobulin-like receptor (KIR), lymphocyte activation gene 3 (LAG3), NOX2, VTCN1 (B7-H4), PD-1, PD-L1, PD-L2, T-cell immunoglobulin and mucin domain-3 (TIM3), CD328 (SIGLEC7), CD329 (SIGLEC9), and T-cell immunoreceptor with Ig and ITIM domains (TIGIT).

B. PD-1 Antagonists

In particular aspects of the present disclosure, the immune checkpoint inhibitor is a PD-1 antagonist. PD-1, programmed cell death 1, is a cell surface receptor that plays a role in suppressing T cell responses in vivo. A B7 homolog known as programmed death ligand 1 (PD-L1) is a natural ligand for PD-1 and delivers its T cell suppression signal by binding to the PD-1 receptor. Most normal human tissues do not express PD-L1 on the cell surface. Human cancers, however, express large amounts of PD-L1 on the cell surface. Engagement of PD-L1 expressed on the surface of tumor cells with PD-1 on the surface of T cells can result in T cell apoptosis, T cell exhaustion, T cell anergy, T cell IL-10 production, and dendritic cell suppression. These signals result in suppressing anti-tumor T cell activity and act as an immunological shield and aid the tumor cell in evading the antitumor immune response.

PD-1 antagonists can prevent PD-1 signaling by binding directly to PD-1 and inhibiting its interaction with PD-L1. This reduces signaling from the PD-1 receptor and blocks PD-1 mediated T cell suppression. In one embodiment, a PD-1 antagonist useful for the disclosure is an anti-PD-1 antibody. In another embodiment, an anti-PD-1 antibody is a monoclonal antibody, a humanized antibody, a human antibody, a single chain antibody, or a chimeric antibody. In another embodiment, an anti-PD-1 antibody for the therapy comprises Fab, F(ab)2, Fv, or scFv.

In some aspects, the PD-1 antagonist is an anti-PD-1 antibody selected from the group consisting of nivolumab (OPDIVO®; see, e.g., U.S. Pat. No. 8,008,449, and Wang et al., 2014, Cancer Immunol Res. 2(9):846-56)); pembrolizumab (KEYTRUDA®; see, e.g., U.S. Pat. Nos. 8,354,509 and 8,900,587); camrelizumab (SHR-1210; see, e.g., Huang et al., Clin Cancer Res. 2018, PMID: 29358502); cemiplimab (LIBTAYO®; see, e.g., US Patent Application Publication No. 2015/0203579A1), sintilimab (TYVYT; see, e.g., Liu et al., J Hematol Oncol., 2017;10(1):136) and PDR001 (see, e.g., Naing et al., J Clin Oncol. 2016).

In some embodiments, the immune checkpoint inhibitor is an anti-PD-1 antibody comprising 3 CDRs of the V_{H} of nivolumab. In some embodiments, the immune checkpoint inhibitor is an anti-PD-1 antibody comprising 3 CDRs of the V_{L} of nivolumab. In other embodiments, the immune checkpoint inhibitor is an anti-PD-1 antibody comprising 3 CDRs of the V_{H} of nivolumab and 3 CDRs of the V_{L} of nivolumab: V_{H} CDR1, V_{H} CDR2, V_{H} CDR3, V_{L} CDR1, V_{L} CDR2, and V_{L} CDR3. In other embodiments, the immune checkpoint inhibitor is an anti-PD-1 antibody comprising a V_{H} comprising the amino acid sequence of the V_{H} of nivolumab and a V_{L} comprising the amino acid sequence of the V_{L} of nivolumab. In some embodiments, the immune checkpoint inhibitor is nivolumab.

Another type of PD-1 antagonist that can reduce or inhibit PD-1 signaling is a molecule binding to PD-L1 and thus blocking PD-L1 interaction with PD-1. This interference of receptor/ligand binding reduces signaling from the PD-1 receptor and blocks PD-1 mediated T cell suppression. In one embodiment, the PD-1 antagonist useful in the present disclosure is an anti-PD-L1 antibody. In another embodiment, the anti-PD-L1 antibody is a monoclonal antibody, a humanized antibody, a human antibody, a single chain antibody, or a chimeric antibody. In another embodiment, the anti-PD-L1 antibody comprises Fab, F(ab)₂, Fv, or scFv.

In some aspects, the PD-1 antagonist is an anti-PD-L1 antibody selected from the group consisting of atezolizumab (TECENTRIQ®; see, e.g., U.S. Pat. No. 8,217,149), avelumab (BAVENCIO®; see, e.g., US 2014/0341917A1), durvalumab (IMFINZI ; see, e.g., US 2013/0034559 A1), and BMS-936559 (see, e.g., U.S. Pat. No. 7,943,743; WO 2013/173223).

C. Combination therapy comprising a Fas-chimera vector and a PD-1 antagonist

The present disclosure provides a method of reducing or inhibiting the size of a tumor or eliminating a tumor in a subject in need thereof comprising (a) administering to the subject an effective dose of a vector comprising a Fas-chimera gene operably linked to an endothelial cell-specific promoter and (b) administering to the subject an effective dose of a PD-1 antagonist. In some aspects, the subject's tumor size is reduced or inhibited, or the tumor in the subject is eliminated, compared to a tumor in a subject without administration of the vector.

The present disclosure also provides a method of treating a tumor or a metastasis thereof in a subject in need thereof comprising (a) administering to the subject an effective dose of a vector comprising a Fas-chimera gene operably linked to an endothelial cell-specific promoter and (b) administering to the subject an effective dose of a PD-1 antagonist. In some aspects, the tumor or metastasis thereof in the subject is treated compared to a tumor or metastasis thereof in a subject without administration of the vector.

The present disclosure also provides a method of inducing or improving T cell activation in a subject having a tumor comprising (a) administering to the subject an effective dose of a vector comprising a Fas-chimera gene operably linked to an endothelial cell-specific promoter and (b) administering to the subject an effective dose of a PD-1 antagonist. In some aspects, T cell activation in the subject is induced or improved compared to T cell activation in a subject without administration of the vector.

The present disclosure also provides a method of inducing or improving the efficacy of a PD-1 antagonist in a subject having a tumor comprising (a) administering to the subject an effective dose of a vector comprising a Fas-chimera gene operably linked to an endothelial cell-specific promoter and (b) administering to the subject an effective dose of a PD-1 antagonist. In some aspects, the efficacy of the PD-1 antagonist is induced or improved in the subject compared to the efficacy of the PD-1 antagonist in a subject without administration of the vector.

The present disclosure also provides a method of converting a cold tumor to a hot tumor in a subject in need thereof, comprising (a) administering to the subject an effective dose of a vector comprising a Fas-chimera gene operably linked to an endothelial cell-specific promoter and (b) administering to the subject an effective dose of a PD-1 antagonist. In some aspects, the cold tumor in the subject is converted to a hot tumor compared to a cold tumor in a subject without administration of the vector. For the methods of the present disclosure, an effective dose of the vector is 0.5×10¹³-2×10¹³ virus particles administered every six weeks, and an effective dose of a PD-1 antagonist, e.g., an anti-PD-1 antibody, e.g., nivolumab, is about 240 mg every two weeks or about 480 mg every four weeks. In some aspects, an anti-PD-1 antibody, e.g., pembrolizumab, is administered at a dose of about 200 mg every three weeks.

Tumor growth can be measured by techniques known in the art, including but not limited to magnetic resonance imaging (MM) scan, functional MM (fMRI) scan, computerized tomography (CT) scan, or positron emission tomography (PET) scan. In a particular aspect, the growth of the tumor is measured by MRI. In some aspects, the tumor of the subject is a recurrent tumor that arose during treatment with the vector. In yet other embodiments, the tumor of the subject is a metastatic tumor that arose during treatment with the vector.

The term “subject” or “individual” or “animal” or “patient” or “mammal,” is meant any subject, particularly a mammalian subject, having or being expected to have an increased or improved anti-tumor response as a result of the present disclosure. In some aspects, the term “subject” or “individual” or “animal” or “patient” or “mammal,” is meant any subject, particularly a mammalian subject, having been administered a combination regimen comprising a vector expressing a Fas chimera protein and a PD-1 antagonist. In one embodiment, the subject is a human. In another embodiment, the subject is a cancer patient.

In certain aspects, the PD-1 antagonist is administered prior to administering the vector, concomitantly with administration of a vector, or after administration of a vector. In other aspects, the vector is administered prior to the PD-1 antagonist for at least one day earlier, at least two days earlier, at least three days earlier, at least four days earlier, at least five days earlier, at least six days earlier, at least seven days earlier, at least nine days earlier, at least 10 days earlier, at least two weeks earlier, at least three weeks earlier, at least four weeks earlier, at least one month earlier, at least two months earlier, or more. In other aspects, the PD-1 antagonist is administered prior to the vector for at least one day earlier, at least two days earlier, at least three days earlier, at least four days earlier, at least five days earlier, at least six days earlier, at least seven days earlier, at least nine days earlier, at least 10 days earlier, at least two weeks earlier, at least three weeks earlier, at least four weeks earlier, at least one month earlier, at least two months earlier, or more.

In some aspects, the tumor of the subject is a recurrent tumor that arose during treatment with the vector. In yet other embodiments, the tumor of the subject is a metastatic tumor that arose during treatment with the vector. In some aspects, the vector is administered prior to the PD-1 antagonist, and the PD-1 antagonist is administerd upon tumor progression. In some aspects, the vector is administered prior to the PD-1 antagonist, and the PD-1 antagonist is administerd upon tumor recurrence.

The effective dose or doses of the vector administered as part of the present disclosure can be measured in virus particles (VPs). In some embodiments, the effective dose of the vector includes but is not limited to equal to or less than about 1×10¹⁶, 1×10¹⁵, 1×10¹⁴, 5×10¹¹, 4×10¹¹, 3×10¹¹, 2×10¹¹, 1×10¹¹, 9×10¹², 8×10¹², 7×10¹², 6×10¹², 5×10¹², 4×10¹², 3×10¹², 2×10¹², 1×10¹², 9×10¹¹, 8×10¹¹, 7×10¹¹, 6×10¹¹, 5×10¹¹, 4×10¹¹, 3×10¹¹, 2×10¹¹, 1×10¹¹, 9×10¹⁰, 8×10¹⁰, 7×10¹⁰, 6×10¹⁰, 5×10¹⁰, 4×10¹⁰, 3×10¹⁰, 2×10¹⁰, or 1×10¹⁰ virus particles. In other embodiments, an effective dose of the vector is about 1×10¹⁰ to about 1×10¹⁶, about 1×10¹¹ to about 1×10¹⁵, about 1×10¹¹ to about 1×10¹⁶, about 1×10¹² to about 1×10¹⁵, about 1×10¹² to about 1×10¹⁶, about 1×10¹² to about 1×10¹⁴, about 5×10¹² to about 1×10¹⁶, about 5×10¹² to about 1×10¹⁵, about 5×10¹² to about 1×10¹⁴, about 1×10¹² to about 1×10¹³, about 1×10¹³ to about 1×10¹⁴ virus particles.

In some aspects, the vector is administered at an effective dose of at least about 1×10¹¹ virus particles. In some aspects, the vector is administered at an effective dose of at least about 1×10¹² virus particles. In some aspects, the vector is administered at an effective dose of at least about 1×10¹³ virus particles. In some aspects, the vector is administered at an effective dose of at least about 1×10¹⁴ virus particles. In some aspects, the vector is administered at an effective dose of at least about 1×10¹⁵ virus particles. In some aspects, the vector is administered at an effective dose of at least about 1×10¹⁶ virus particles. In some aspects, the vector is administered at an effective dose of at least about 1×10⁷, 1×10⁸, 1×10⁹, 1×10¹⁰, or 5×10¹⁰ virus particles.

In some aspects of the disclosure, an effective dose of the PD-1 antagonist is administered as a flat dose. The use of the term “flat dose” with regard to the present disclosure means a dose that is administered to a patient without regard for the weight or body surface area (BSA) of the patient. The flat dose is therefore not provided as a mg/kg dose, but rather as an absolute amount of the agent (e.g., the anti-PD-1 antibody). For example, a 60 kg person and a 100 kg person would receive the same dose of the composition (e.g., 240 mg of an anti-PD-1 antibody).

In some aspects, the PD-1 antagonist is an anti-PD-1 or an anti-PD-L1 antibody. In some aspects, the effective dose of the anti-PD-1 or anti-PD-L1 antibody is a dose (e.g., flat dose) of between about 100 mg to about 600 mg. In some aspects, the effective dose of the anti-PD-1 or PD-L1 antibody is a flat dose of about 100-300 mg, such as, about 200-300 mg, about 220-260 mg, about 230-250 mg or about 240 mg. In some aspects, the effective dose of the anti-PD-1 or anti-PD-L1 antibody is a flat dose of about 400-600 mg, such as about 450-520 mg, about 460-510 mg, about 470-500 mg, or about 480 mg. In some aspects, the effective dose of the anti-PD-1 or PD-L1 antibody is a dose (e.g., flat dose), such as about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, or about 600 mg.

In some aspects, the effective dose of the anti-PD-1 antibody or anti-PD-L1 antibody is a dose (e.g., flat dose) of between about 60-100 mg, about 60-200 mg, about 60-300 mg, about 60-400 mg, about 60-500 mg, or about 60-600 mg. In some aspects, the effective dose of the anti-PD-1 antibody or anti-PD-L1 antibody is a dose (e.g., flat dose) of between about 100-200 mg, about 100-300 mg, about 100-400 mg, or about 100-500 mg. In some aspects, the effective dose of the anti-PD-1 antibody or anti-PD-L1 antibody is a dose (e.g., flat dose) of between about 300-400 mg or about 300-500 mg. In some aspects, the effective dose of the anti-PD-1 antibody or anti-PD-L1 antibody is a dose (e.g., flat dose) of between about 400-500 mg. In a particular aspect, the effective dose of the anti-PD-1 antibody or anti-PD-L1 antibody is a dose (e.g., flat dose) of about 480 mg.

The term “weight based dose” as referred to herein means that a dose that is administered to a patient is calculated based on the weight of the patient. For example, when a patient with 60 kg body weight requires 3 mg/kg of an anti-PD-1 antibody, one can draw the appropriate amounts of the anti-PD-1 antibody (i.e., 180 mg).

In some aspects, the effective dose of the PD-1 antagonist is a weight-based dose equal to or less than about 15 mg/kg, 14 mg/kg, 13 mg/kg, 12 mg/kg, 11 mg/kg, 10 mg/kg, 9 mg/kg, 8 mg/kg, 7 mg/kg, 6 mg/kg, 5 mg/kg, 4 mg/kg, 3 mg/kg, 2 mg/kg, or 1 mg/kg. In a particular embodiment, the effective dose of the PD-1 antagonist is about 3 mg/kg.

In some aspects, the PD-1 antagonist is nivolumab. In some aspects, the vector is administered at an effective dose of 3×10¹² to 3×10¹³ VPs, and nivolumab is administered at an effective dose (weight based dose) of 2 mg/kg to 12 mg/kg. In other aspects, the vector is administered at an effective dose of 3×10¹² to 3×10¹³ VPs, and nivolumab is administered at an effective dose (flat dose) of 460 mg to 500 mg.

In a particular aspect, the effective dose of the vector is administered at an amount of 1×10¹³ VPs, and the effective dose of nivolumab is administered at an amount (weight based dose) of 3 mg/kg. In another aspect, the effective dose of the vector is administered at an amount of 3×10¹² to 1×10¹³ VPs, and the effective dose of nivolumab is administered at an amount (flat dose) of 200 mg to 260 mg. In a particular aspect, the effective dose of the vector is administered at an amount of 1×10¹³ VPs, and nivolumab is administered at an effective amount (flat dose) of 240 mg.

In another aspect, the effective dose of the vector is administered at an amount of 1×10¹³ VPs, and the effective dose of nivolumab is administered at an amount (weight based dose) of 3 mg/kg to 12 mg/kg. In another aspect, the effective dose of the vector is administered at an amount of 3×10¹² to 1×10¹³ VPs, and the effective dose of nivolumab is administered at an amount (flat dose) of 460 mg to 500 mg. In a particular aspect, the effective dose of the vector is administered at an amount of 1×10¹³ VPs, and the effective dose of nivolumab is administered at an amount (flat dose) of 480 mg.

In some aspects, the PD-1 antagonist is pembrolizumab. In some aspects, the vector is administered at an effective dose of 3×10¹² to 3×10¹³ VPs, and pembrolizumab is administered at an effective dose (flat dose) of 150 mg to 250 mg.

In another aspect, the effective dose of the vector is administered at an amount of 3×10¹² to 1×10¹³ VPs, and the effective dose of pembrolizumab is administered at an amount (flat dose) of 180 mg to 220 mg. In a particular aspect, the effective dose of the vector is administered at an amount of 1×10¹³ VPs, and pembrolizumab is administered at an effective amount (flat dose) of 200 mg.

The methods of the present disclosure comprise administering at least one effective dose of the vector, and a PD-1 antagonist. The regimen used for administering the vector and the PD-1 antagonist comprises repeated administration of the vector and the PD-1 antagonist. In one embodiment, the vector is repeatedly administered every day, once in about 2 days, once in about 3 days, once in about 4 days, once in about 5 days, once in about 6 days, once in about 7 days, once in about 2 weeks, once in about 3 weeks, once in about 4 weeks, once in about 5 weeks, once in about 6 weeks, once in about 7 weeks, once in about 2 months, or once in about 6 months. In another embodiment the PD-1 antagonist is repeatedly administered once in about 7 days, once in about 2 weeks, once in about 3 weeks, once in about 4 weeks, once in about 2 months, once in about 3 months, once in about 4 months, once in about 5 months, or once in about 6 months. In a particular embodiment, the vector is administered every 2 months and the PD-1 antagonist is administered every 2 weeks. In a specific embodiment, the PD-1 antagonist is nivolumab.

D. Diseases and Conditions

The methods of the present disclosure are useful for reducing or inhibiting the size of a tumor or eliminating a tumor in a subject in need thereof. In some aspects of the present disclosure, the tumor is derived from or associated with metastatic colorectal cancer (mCRC), advanced nonsquamous non-small cell lung cancer (NSCLC), metastatic renal cell carcinoma (mRCC), glioblastoma multiforme (GBM), Mullerian cancer, ovarian cancer, peritoneal cancer, fallopian tube cancer, or uterine papillary serous carcinoma. In some aspects, the methods of the present disclosure reduce the volume of malignant peritoneal fluid, e.g., ascites, reduces pain to the subject, prolongs survival of the subject, or any combinations thereof. The tumor that can be reduced, inhibited, or treated with the combination of the vector and the PD-1 antagonist. The tumor can be a solid tumor, a primary tumor, a metastatic tumor, or any combination thereof. The term “metastatic” or “metastasis” refers to tumor cells that are able to establish secondary tumor lesions in another parts or organ.

In the methods of the present disclosure, a “solid tumor” includes, but is not limited to, sarcoma, melanoma, carcinoma, or other solid tumor cancer. “Sarcoma” refers to a tumor which is made up of a substance like the embryonic connective tissue and is generally composed of closely packed cells embedded in a fibrillar or homogeneous substance. Sarcomas include, but are not limited to, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” refers to a tumor arising from the melanocytic system of the skin and other organs. Melanomas include, for example, acra-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, metastatic melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidernoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma viflosum.

Additional cancers that can be inhibited or treated according to the present methods include, for example, Leukemia, Hodgkin's Disease, Non-Hodgkin's Lymphoma, multiple myeloma, neuroblastoma, breast cancer, ovarian cancer, lung cancer, (including non-small cell lung cancer (NSCLC)), rhabdomyosarcoma, primary thrombocytosis, primary macroglobulinemia, small-cell lung tumors, primary brain tumors, gliomas (including glioblastoma multiforme (GBM) and recurrent GBM), gastrointestinal (GI) cancers (including but not limited to cancers of the esophagus, gallbladder, biliary tract, liver, pancreas, stomach, small intestine, large intestine, colon, rectum, and anus), malignant pancreatic insulanoma, malignant carcinoid, urinary bladder cancer, premalignant skin lesions, testicular cancer, lymphomas, thyroid cancer, papillary thyroid cancer, neuroblastoma, neuroendocrine cancer, genitourinary tract cancer, malignant hypercalcemia, cervical cancer, endometrial cancer, adrenal cortical cancer, prostate cancer, Mullerian cancer, ovarian cancer, peritoneal cancer, fallopian tube cancer, or uterine papillary serous carcinoma.

In some aspects, the tumor is a recurrent tumor. In some aspects, the tumor is a metastatic tumor.

### III. Nucleic Acid Constructs Comprising a Fas-chimera Gene and an Endothelial Cell Specific Promoter

The present disclosure provides methods of anti-tumor therapy comprising (a) administering to the subject an effective dose of a vector comprising a Fas-chimera gene operably linked to an endothelial cell-specific promoter and (b) administering to the subject an effective dose of a checkpoint inhibitor.

The gene encoding the Fas-chimera protein (or gene product), in the present disclosure can be linked to an endothelial cell-specific promoter, which directs expression of the Fas-chimera gene product in an endothelial cell. Expression of such a cytotoxic gene product is useful in a situation where excessive neo-vascularization or blood vessel growth is not desirable, e.g., in a tumor.

A. Fas-Chimera

A Fas-chimera protein expressed by the nucleic acid construct of the disclosure comprises at least two “death receptor” polypeptides, each of the polypeptides is derived from a different protein. The first polypeptide of the Fas -chimera protein comprises a ligand binding domain of Tumor Necrosis Factor Receptor 1 (TNFR1). The second polypeptide of the Fas-chimera protein comprises an effector domain of a Fas polypeptide.

The ligand binding domain of TNFR1 can be any domain that binds to a TNFR1 ligand. In one embodiment, the TNFR1 ligand is TNF-α. In another embodiment, the TNFR1 ligand is lymphotoxin-a. The ligand binding domain of TNFR1 can be an extracellular domain of TNFR1 or any fragments, variants, derivatives, or analogues thereof. Non-limiting examples of the TNFR1 ligand binding domain are described below.

The effector domain of a Fas polypeptide useful for the disclosure comprises any Fas domains that form death-inducing signaling complex (DISC), thereby inducing apoptosis. In one embodiment, an effector domain of a Fas polypeptide comprises an intracellular domain, a trans-membrane domain, or both. Non-limiting examples of Fas polypeptide effector domains are described below.

The TNFR1 and the Fas polypeptide can be linked by a peptide bond or by a linker. The linker connecting the TNFR1 ligand binding domain with the Fas effector domain can be a polypeptide linker or a non-peptide linker. For example, a linker for the Fas-chimera protein can comprise one or more glycine, serine, leucine, or any combinations thereof. In one embodiment, a linker useful for the disclosure comprises Ser-Leu. In another embodiment, a linker useful for the disclosure comprises (GGGS)n, (Denise et al. J. Biol. Chem. 277:35035-35043 (2002)), wherein n can be 0, 1, 2, 3, 4, 5, 6, 7, 8, 9, or 10 or more (SEQ ID NO: 25).

1. Tumor Necrosis Factor Receptor 1

The full-length human TNFR1 polypeptide is 455 amino acids in length and is also known as TNF-R1, Tumor necrosis factor receptor type I (TNFRI), TNFR-I, TNFRSF1A, TNFAR, p55, P60, or CD120a. Naturally-occurring human TNFR1 polypeptide is known to bind to TNF-α or homotrimeric lymphotoxin-α. Binding of TNF-α to the extracellular domain leads to homotrimerization of TNFR1, which then interacts specifically with the death domain of Tumor Necrosis Factor Receptor Type 1-Associated Death Domain Protein (TRADD). Various TRADD-interacting proteins such as TNF Receptor Associated Factors (TRAFS), Receptor-Interacting Serine/Threonine-Protein Kinase 1 (RIPK1), and Fas-Associated Protein with Death Domain (FADD) are recruited to the complex by their association with TRADD. The complex activates at least two distinct signaling cascades, apoptosis and NF-kappa-B signaling.

A 455 aa polypeptide sequence reported as a human TNFR1 polypeptide sequence has the identifier number P19438-1 in the UniProtKB database. This human TNFR1 polypeptide sequence is designated herein as isoform A and SEQ ID NO: 2. SEQ ID NO: 1 is a nucleotide sequence encoding SEQ ID NO: 2. A polypeptide sequence of 108 aa was reported as an isoform of the human TNFR1 polypeptide sequence and has the identifier number P19438-2 in the UniProtKB database. The 108 aa polypeptide corresponds to amino acids 1 to 108 of isoform A (SEQ ID NO: 2) and is designated herein as isoform B. Another variant of the human TNFR1 polypeptide having 232 aa was reported as the identifier number P19438-3 in the UniProtKB database. The 232 aa polypeptide corresponds to amino acids 1 to 232 of isoform A (SEQ ID NO: 2) and is designated herein as isoform C. Additional natural variants of human TNFR1 include, but are not limited to, the TNFR1 polypeptide of isoforms A, B, and C comprising one or more mutations selected from the group consisting of H51Q, C59R, C59S, C62G, C62Y, P75L, T79M, C81F, C99S, S115G, C117R, C117Y, R121P, R121Q, P305T, and any combinations thereof. Other known TNFR1 variants include the TNFR1 polypeptide of isoforms A, B, and C comprising L13LILPQ, K255E, S286G, R394L, 412:Missing, GPAA443-446APP, or any combinations thereof.

Table 1 shows the human wild-type TNFR1 amino acid sequence and a nucleotide sequence encoding the wild-type TNFR1.

TABLE 1
TNFR1 Sequences

| SEQ ID No. | Sequences |
|---|---|
| Amino acid | MGLSTVPDLLLPLVLLELLVGIYPSGVIGLVPHLGDREKRDSVCPQGKYIHPQNNSICCT |
| sequence of TNFR1 | KCHKGTYLYNDCPGPGQDTDCRECESGSFTASENHLRHCLSCSKCRKEMGQVEISSCTVD |
| (SEQ ID NO: 2) | RDTVCGCRKNQYRHYWSENLFQCFNCSLCLNGTVHLSCQEKQNTVCTCHAGEFLRENECV |
| | SCSNCKKSLECTKLCLPQIENVKGTEDSGTTVLLPLVIFFGLCLLSLLFIGLMYRYQRWK |
| | SKLYSIVCGKSTPEKEGELEGTTTKPLAPNPSFSPTPGFTPTLGFSPVPSSTFTSSSTYT |
| | PGDCPNFAAPRREVAPPYQGADPILATALASDPIPNPLQKWEDSAHKPQSLDTDDPATLY |
| | AVVENVPPLRWKEFVRRLGLSDHEIDRLELQNGRCLREAQYSMLATWRRRTPRREATLEL |
| | LGRVLRDMDLLGCLEDIEEALCGPAALPPAPSLLR |
| Nucleotide Sequence | atgggcctctccaccgtgcctgacctgctgctgccgctggtgctcctggagctgttggtg |
| encoding TNFR1 | ggaatatacccctcaggggttattggactggtccctcacctaggggacagggagaagaga |
| (SEQ ID NO: 1) | gatagtgtgtgtccccaaggaaaatatatccaccctcaaaataattcgatttgctgtacc |
| | aagtgccacaaaggaacctacttgtacaatgactgtccaggcccggggcaggatacggac |
| | tgcagggagtgtgagagcggctccttcaccgcttcagaaaaccacctcagacactgcctc |
| | agctgctccaaatgccgaaaggaaatgggtcaggtggagatctcttcttgcacagtggac |
| | cgggacaccgtgtgtggctgcaggaagaaccagtaccggcattattggagtgaaaacctt |
| | ttccagtgcttcaattgcagcctctgcctcaatgggaccgtgcacctctcctgccaggag |
| | aaacagaacaccgtgtgcacctgccatgcaggtttctttctaagagaaaacgagtgtgtc |
| | tcctgtagtaactgtaagaaaagcctggagtgcacgaagttgtgcctaccccagattgag |
| | aatgttaagggcactgaggactcaggcaccacagtgctgttgcccctggtcattttcttt |
| | ggtctttgccttttatccctcctcttcattggtttaatgtatcgctaccaacggtggaag |
| | tccaagctctactccattgtttgtgggaaatcgacacctgaaaaagagggggagcttgaa |
| | ggaactactactaagcccctggccccaaacccaagcttcagtcccactccaggcttcacc |
| | cccaccctgggcttcagtcccgtgcccagttccaccttcacctccagctccacctatacc |
| | cccggtgactgtcccaactttgcggctccccgcagagaggtggcaccaccctatcagggg |
| | gctgaccccatccttgcgacagccctcgcctccgaccccatccccaacccccttcagaag |
| | tgggaggacagcgcccacaagccacagagcctagacactgatgaccccgcgacgctgtac |
| | gccgtggtggagaacgtgcccccgttgcgctggaaggaattcgtgcggcgcctagggctg |
| | agcgaccacgagatcgatcggctggagctgcagaacgggcgctgcctgcgcgaggcgcaa |
| | tacagcatgctggcgacctggaggcggcgcacgccgcggcgcgaggccacgctggagctg |
| | ctgggacgcgtgctccgcgacatggacctgctgggctgcctggaggacatcgaggaggcg |
| | ctttgcggccccgccgccctcccgcccgcgcccagtcttctcaga |
| Amino acid | MGLSTVPDLLLPLVLLELLVGIYPSGVIGLVPHLGDREKRDSVCPQGKYIHPQNNSICCT |
| sequence of a | KCHKGTYLYNDCPGPGQDTDCRECESGSFTASENHLRHCLSCSKCRKEMGQVEISSCTVD |
| Ligand Binding | RDTVCGCRKNQYRHYWSENLFQCFNCSLCLNGTVHLSCQEKQNTVCTCHAGEFLRENECV |
| Domain of TNFR1 | SCSNCKKSLECTKLCLP |
| (SEQ ID NO: 4) | |
| Nucleotide sequence | atgggcctct ccaccgtgcc tgacctgctg ctgccgctgg tgctcctgga |
| encoding a Ligand | gctgttggtg ggaatatacc cctcaggggt tattggactg gtccctcacc |
| Binding Domain of | taggggacag ggagaagaga gatagtgtgt gtccccaagg aaaatatatc |
| TNFR1 | caccctcaaa ataattcgat ttgctgtacc aagtgccaca aaggaaccta |
| (SEQ ID NO: 3) | cttgtacaat gactgtccag gcccggggca ggatacggac tgcagggagt |
| | gtgagagcgg ctccttcacc gcttcagaaa accacctcag acactgcctc |
| | agctgctcca aatgccgaaa ggaaatgggt caggtggaga tctcttcttg |
| | cacagtggac cgggacaccg tgtgtggctg caggaagaac cagtaccggc |
| | attattggag tgaaaacctt ttccagtgct tcaattgcag cctctgcctc |
| | aatgggaccg tgcacctctc ctgccaggag aaacagaaca ccgtgtgcac |
| | ctgccatgca ggtttctttc taagagaaaa cgagtgtgtc tcctgtagta |
| | actgtaagaa aagcctggag tgcacgaagt tgtgcctacc a |

The mouse TNFR1 polypeptide sequence and its variants are also reported. The 454 aa mouse TNFR1 polypeptide has the identifier number P25118 in UniProtKB database. TNFR1 polypeptides known in other animals include, but are not limited to, rat (e.g., P22934 in the UniProtKB database), cow (e.g., O19131 in the UniProtKB database), pig (e.g., P50555 in the UniProtKB database), or horse (e.g., D1MH71 in the UniProtKB database).

The full-length TNFR1 can be cleaved into two chains, (1) TNF Receptor Superfamily Member 1A, membrane form (i.e., amino acids 22 to 455 corresponding to full-length TNFR1) and (2) TNF-binding protein 1 (TBPI) (i.e., amino acids 41 to 291 corresponding to full-length TNFR1). The full-length human TNFR1 polypeptide consists of a signal sequence (amino acids 1 to 21 of SEQ ID NO: 2), an extracellular domain (amino acids 22 to 211 of SEQ ID NO: 2), a trans-membrane domain (amino acids 212 to 234 of SEQ ID NO: 2), and a cytoplasmic domain (amino acids 235 to 455 of SEQ ID NO: 2). The TNFR1 extracellular domain comprises four cysteine repeat regions, TNFR-Cys1 (amino acids 43 to 82 corresponding to SEQ ID NO: 2), TNFR-Cys2 (amino acids 83 to 125 corresponding to SEQ ID NO: 2), TNFR-Cys3 (amino acids 126 to 166 corresponding to SEQ ID NO: 2), and TNFR-Cys4 (amino acids 167 to 196 corresponding to SEQ ID NO: 2).

As one of skill in the art will appreciate, the beginning and ending residues of the domains listed above can vary depending upon the computer modeling program used or the method used for determining the domain. As such, various functional domains of TNFR1 can vary from those defined above.

In one embodiment, a ligand binding domain of TNFR1 useful for the Fas-chimera protein comprises, consists essentially of, or consists of an extracellular domain of TNFR1, or any fragment, variant, derivative, or analogue thereof, wherein the extracellular domain of TNFR1, or any fragment, variant, derivative, or analogue thereof binds to TNF-α. In another embodiment, a ligand binding domain of TNFR1 comprises TNFR-Cys1; TNFR-Cys2; TNFR-Cys3; TNFR-Cys4; TNFR-Cys1 and TNFR-Cys2; TNFR-Cys1 and TNFR-Cys3; TNFR-Cys1 and TNFR-Cys4; TNFR-Cys2 and TNFR-Cys3; TNFR-Cys2 and TNFR-Cys4; TNFR-Cys3 and TNFR-Cys4; TNFR-Cysl, TNFR-Cys2, and TNFR-Cys3; TNFR-Cysl, TNFR-Cys2, and TNFR-Cys4; TNFR-Cys2, TNFR-Cys3, and TNFR-Cys4; or TNFR-Cysl, TNFR-Cys2, TNFR-Cys3, and TNFR-Cys4. In other embodiments, a ligand binding domain of TNFR1 in the Fas-chimera protein comprises TNF binding protein I. In yet other embodiments, a TNFR1 ligand binding domain of the Fas-chimera protein comprises, consists essentially of, or consists of an amino acid sequence at least 60%, 70%, 80%, 90%, 95%, 96%, 97%, 98%, 99%, or 100% identical to amino acids 22 to 190, amino acids 22 to 191, amino acids 22 to 192, amino acids 22 to 193, amino acids 22 to 194, amino acids 22 to 195, amino acids 22 to 196, amino acids 22 to 197, amino acids 22 to 198, amino acids 22 to 199, amino acids 22 to 200, amino acids 22 to 201, amino acids 22 to 202, amino acids 22 to 203, amino acids 22 to 204, amino acids 22 to 205, amino acids 22 to 206, amino acids 22 to 207, amino acids 22 to 208, amino acids 22 to 209, amino acids 22 to 210, or amino acids 22 to 211 of SEQ ID NO: 2, wherein the ligand binding domain binds to a TNFR1 ligand, e.g., TNF-α.

In other embodiments, the ligand binding domain of TNFR1 further comprises a signal peptide. One example of the suitable signal peptides is the signal peptide of TNFR1, e.g., amino acids 1 to 21 of SEQ ID NO: 2. In yet other embodiments, a ligand binding domain of the Fas-chimera gene product comprises, consists essentially of, or consists of an amino acid sequence at least 60%, 70%, 80%, 90%, 95%, 96%, 97%, 98%, 99%, or 100% identical to amino acids 1 to 190, amino acids 1 to 191, amino acids 1 to 192, amino acids 1 to 193, amino acids 1 to 194, amino acids 1 to 195, amino acids 1 to 196, amino acids 1 to 197, amino acids 1 to 198, amino acids 1 to 199, amino acids 1 to 200, amino acids 1 to 201, amino acids 1 to 202, amino acids 1 to 203, amino acids 1 to 204, amino acids 1 to 205, amino acids 1 to 206, amino acids 1 to 207, amino acids 1 to 208, amino acids 1 to 209, amino acids 1 to 210, or amino acids 1 to 211 of SEQ ID NO: 2, wherein the ligand binding domain binds to a TNFR1 ligand, e.g., TNF-α. In a specific embodiment, a TNFR1 ligand binding domain of the Fas-chimera protein comprises, consists essentially of, or consists of an amino acid sequence at least 60%, 70%, 80%, 90%, 95%, 96%, 97%, 98%, 99%, or 100% identical to SEQ ID NO: 4, wherein the ligand binding domain binds to a TNFR1 ligand, e.g., TNF-α.

In yet other embodiments, the ligand binding domain of TNFR1 is encoded by a nucleotide sequence at least 60%, 70%, 80%, 90%, 95%, 96%, 97%, 98%, 99%, or 100% identical to SEQ ID NO: 3.

In still other embodiments, a TNFR1 ligand binding domain of the Fas-chimera protein comprises, consists essentially of, or consists of an amino acid sequence at least 60%, 70%, 80%, 90%, 95%, 96%, 97%, 98%, 99%, or 100% identical to amino acids 22 to 108 of SEQ ID NO: 2 (TNFR1 isoform B), amino acids 22 to 232 of SEQ ID NO: 2 (TNFR1 isoform C), or amino acids 44 to 291 of SEQ ID NO: 2 (TBP1), wherein the ligand binding domain binds to a TNFR1 ligand, e.g., TNF-α.

2. Fas Polypeptide

The full-length human Fas polypeptide is 335 amino acids in length and is also known as Tumor Necrosis Factor Receptor Superfamily Member 6, Apo-1 antigen, Apoptosis-mediating surface antigen Fas, FasLG receptor, or CD95. Naturally occurring Fas polypeptide is a receptor for TNFSF6/FasLG. When the Fas polypeptide binds to the Fas ligand (FasL), the interaction between Fas and FasL results in the formation of the death-inducing signaling complex (DISC), which contains the FADD, caspase-8 and caspase-10. In some types of cells (type I), processed caspase-8 directly activates other members of the caspase family, and triggers the execution of apoptosis of the cell. In other types of cells (type II), the Fas-DISC starts a feedback loop that spirals into increasing release of proapoptotic factors from mitochondria and the amplified activation of caspase-8. Fas-mediated apoptosis can have a role in the induction of peripheral tolerance, in the antigen-stimulated suicide of mature cells or both.

A 335 aa polypeptide sequence reported as a human Fas polypeptide sequence has the identifier number P25445-1 in the UniProtKB database. This human Fas polypeptide sequence is designated herein as SEQ ID NO: 6. SEQ ID NO: 5 is a nucleotide sequence encoding SEQ ID NO: 6. The nucleotide sequence encoding the Fas polypeptide is also known as APT1, FAS1, or TNFRSF6. The full-length Fas polypeptide contains a signal peptide (amino acids 1 to 25 corresponding to SEQ ID NO: 6), an extracellular domain (amino acids 26 to 173 corresponding to SEQ ID NO: 6), a trans-membrane domain (amino acids 174 to 190 corresponding to SEQ ID NO: 6), and an intracellular (or cytoplasmic) domain (amino acids 191 to 335 corresponding to SEQ ID NO: 6). The intracellular domain contains a death domain (e.g., amino acids 230 to 314 corresponding to SEQ ID NO: 6).

As one of skill in the art will appreciate, the beginning and ending residues of the domains listed above can vary depending upon the computer modeling program used or the method used for determining the domain. As such, various functional domains of Fas can vary from those defined above. Table 2 shows the wild-type human Fas amino acid sequence and a nucleotide sequence encoding the Fas protein.

TABLE 2
Fas Sequences

| | Sequences |
|---|---|
| Amino acid | MLGIWTLLPLVLTSVARLSSKSVNAQVTDINSKGLELRKTVTTVETQNLEGLHHDGQFCH |
| Sequence of | KPCPPGERKARDCTVNGDEPDCVPCQEGKEYTDKAHFSSKCRRCRLCDEGHGLEVEINCT |
| Human Fas | RTQNTKCRCKPNFFCNSTVCEHCDPCTKCEHGIIKECTLTSNTKCKEEGSRSNLGWLCLL |
| protein | LLPIPLIVWVKRKEVQKTCRKHRKENQGSHESPTLNPETVAINLSDVDLSKYITTIAGVM |
| (SEQ ID NO: 6) | TLSQVKGFVRKNGVNEAKIDEIKNDNVQDTAEQKVQLLRNWHQLHGKKEAYDTLIKDLKK |
| | ANLCTLAEKIQTIILKDITSDSENSNFRNEIQSLV |
| Nucleotide | atgctgggcatctggaccctcctacctctggttcttacgtctgttgctagattatcgtcc |
| sequence | aaaagtgttaatgcccaagtgactgacatcaactccaagggattggaattgaggaagact |
| encoding human | gttactacagttgagactcagaacttggaaggcctgcatcatgatggccaattctgccat |
| Fas sequence | aagccctgtcctccaggtgaaaggaaagctagggactgcacagtcaatggggatgaacca |
| (SEQ ID NO: 5) | gactgcgtgccctgccaagaagggaaggagtacacagacaaagcccatttttcttccaaa |
| | tgcagaagatgtagattgtgtgatgaaggacatggcttagaagtggaaataaactgcacc |
| | cggacccagaataccaagtgcagatgtaaaccaaactttttttgtaactctactgtatgt |
| | gaacactgtgacccttgcaccaaatgtgaacatggaatcatcaaggaatgcacactcacc |
| | agcaacaccaagtgcaaagaggaaggatccagatctaacttggggtggctttgtcttctt |
| | cttttgccaattccactaattgtttgggtgaagagaaaggaagtacagaaaacatgcaga |
| | aagcacagaaaggaaaaccaaggttctcatgaatctccaactttaaatcctgaaacagtg |
| | gcaataaatttatctgatgttgacttgagtaaatatatcaccactattgctggagtcatg |
| | acactaagtcaagttaaaggctttgttcgaaagaatggtgtcaatgaagccaaaatagat |
| | gagatcaagaatgacaatgtccaagacacagcagaacagaaagttcaactgcttcgtaat |
| | tggcatcaacttcatggaaagaaagaagcgtatgacacattgattaaagatctcaaaaaa |
| | gccaatctttgtactcttgcagagaaaattcagactatcatcctcaaggacattactagt |
| | gactcagaaaattcaaacttcagaaatgaaatccaaagcttggtctag |
| Amino acid | GSRSNLGWLCLLLLPIPLIVWVKRKEVQKTCRKHRKENQGS |
| Sequence of an | HESPTLNPETVAINLSDVDLSKYITTIAGVMTLSQVKGFVR |
| Effect or Domain | KNGVNEAKIDEIKNDNVQDTAEQKVQLLRNWHQLHGKKEAY |
| of Fas (SEQ ID | DTLIKDLKKANLCTLAEKIQTIILKDITSDSENSNFRNEIQ |
| NO: 8) | SLV |
| Nucleotide | aggatccagatctaacttggggtggctttgtcttcttcttttgccaattccactaatt |
| sequence | gtttgggtgaagagaaaggaagtacagaaaacatgcagaaagcacagaaaggaaaacc |
| encoding an | aaggttctcatgaatctccaaccttaaatcctgaaacagtggcaataaatttatctga |
| Effector Domain | tgttgacttgagtaaatatatcaccactattgctggagtcatgacactaagtcaagtt |
| of Fas (SEQ ID | aaaggctttgttcgaaagaatggtgtcaatgaagccaaaatagatgagatcaagaatg |
| NO: 7) | acaatgtccaagacacagcagaacagaaagttcaactgcttcgtaattggcatcaact |
| | tcatggaaagaaagaagcgtatgacacattgattaaagatctcaaaaaagccaatctt |
| | tgtactcttgcagagaaaattcagactatcatcctcaaggacattactagtgactcag |
| | aaaattcaaacttcagaaatgaaatccaaagcttggtctag |

The mouse Fas polypeptide sequence and its variants are also reported. The 327 aa mouse Fas polypeptide has the identifier number P25446 in UniProtKB database. Fas polypeptides known in other animals include, but are not limited to, Old World monkey (e.g., Q9BDN4in the UniProtKB database), Rhesus monkey (e.g., Q9BDP2in the UniProtKB database), rat (e.g., Q63199in the UniProtKB database), or cow (e.g., P51867in the UniProtKB database).

Based on the sequence variation in the Fas polypeptide, a person of ordinary skill in the art can identify sequence variations in the effector domain of the Fas polypeptide. For example, natural variants of the Fas effector domains can include one or more substitutions or mutations of C178R, L180F, P183L, I184V, T198I, Y232C, T241K, T241P, V249L, R250P, R250Q, G253D, G253S, N255D, A257D, I259R, D260G, D260V, D260Y, I262S, N264K, T270I, T270K, E272G, E272K, L278F, K299N, T305I, I310S, or any combinations thereof.

In one embodiment, an effector domain of the Fas polypeptide useful for the disclosure comprises a death domain of the Fas polypeptide. In another embodiment, an effector domain of the Fas polypeptide comprises, consists essentially of, or consists of an amino acid sequence at least 60%, 70%, 80%, 90%, 95%, 96%, 97%, 98%, 99%, or 100% identical to amino acids 230 to 314 of SEQ ID NO: 6. In other embodiments, an effector domain of the Fas polypeptide comprises an intracellular domain of the Fas polypeptide. In yet other embodiments, an effector domain of the Fas polypeptide comprises an amino acid sequence at least 60%, 70%, 80%, 90%, 95%, 96%, 97%, 98%, 99%, or 100% identical to amino acids 185 to 335, amino acids 186 to 335, amino acids 187 to 335, amino acids 188 to 335, amino acids 189 to 335, amino acids 190 to 335, amino acids 191 to 335, amino acids 192 to 335, amino acids 193 to 335, amino acids 194 to 335, amino acids 195 to 335, amino acids 196 to 335, amino acids 197 to 335, amino acids 198 to 335, or amino acids 199 to 335 of SEQ ID NO: 6.

In still other embodiments, the effector domain of the Fas polypeptide further comprises a trans-membrane domain of the Fas polypeptide. In yet other embodiments, an effector domain of the Fas polypeptide comprises an amino acid sequence at least about 60%, 70%, 80%, 90%, 95%, 96%, 97%, 98%, 99%, or 100% identical to amino acids 174 to 335 of SEQ ID NO: 6. In some embodiments, an effector domain of the Fas polypeptide further comprises about ten, about nine, about eight, about seven, about six, about five, about four, about three, about two, or about one amino acid from the C-terminal portion of the Fas extracellular domain. In certain embodiments, an effector domain of the Fas polypeptide comprises an amino acid sequence at least 60%, 70%, 80%, 90%, 95%, 96%, 97%, 98%, 99%, or 100% identical to amino acids 179 to 335, amino acids 178 to 335, amino acids 177 to 335, amino acids 176 to 335, amino acids 175 to 335, amino acids 174 to 335, amino acids 173 to 335, amino acids 172 to 335, amino acids 171 to 335, amino acids 170 to 335, amino acids 169 to 335, amino acids 168 to 335, amino acids 167 to 335, amino acids 166 to 335, amino acids 165 to 335, amino acids 164 to 335, or amino acids 163 to 335 of SEQ ID NO: 6, wherein the effector domain forms a death-inducing signaling complex (DISC), activates caspase 8, or induces apoptosis.

In some embodiments, an effector domain of the Fas polypeptide comprises, consists essentially of, or consists of an amino acid sequence at least 70%, 80%, 90%, 95%, 96%, 97%, 98%, 99%, or 100% identical to SEQ ID NO: 8, wherein the effector domain forms a death-inducing signaling complex (DISC), activates caspase 8, or induces apoptosis.

In other embodiments, an effector domain of the Fas polypeptide is encoded by a nucleotide sequence at least 60%, 70%, 80%, 90%, 95%, 96%, 97%, 98%, 99%, or 100% identical to SEQ ID NO: 7.

In one embodiment, the Fas-chimera gene product for the disclosure comprises, consists essentially of, or consists of an amino acid sequence at least 70%, 80%, 90%, 95%, 96%, 97%, 98%, 99%, or 100% identical to SEQ ID NO: 10, wherein the Fas-chimera gene product induces apoptosis. In another embodiment, the Fas-chimera gene product is encoded by a nucleotide sequence at least 60%, 70%, 80%, 90%, 95%, 96%, 97%, 98%, 99%, or 100% identical to SEQ ID NO: 9, wherein the Fas-chimera gene product induces apoptosis.

B. Endothelial Cell-Specific Promoter

The nucleic acid construct comprising a Fas-chimera gene further comprises one or more expression control elements useful for regulating the expression of an operably linked Fas-chimera gene. The expression control elements include, but are not limited to, promoters, secretion signals, and other regulatory elements.

The nucleic acid construct useful for the present disclosure utilizes an endothelial cell-specific promoter to direct expression of the Fas-chimera protein in an endothelial cell, thereby inducing apoptosis of the endothelial cell.

For the purpose of the present disclosure, an endothelial cell-specific promoter can contain one or more cis-regulatory elements, which improve the endothelial cell-specificity of the promoters compared to the promoter without the cis-regulatory elements. In some aspects, the cis-regulatory element comprises a hypoxia response element.

In one embodiment, a cis-regulatory element useful for the disclosure comprises a nucleotide sequence at least 60%, 70%, 80%, 90%, 95%, 96%, 97%, 98%, 99%, or 100% identical to SEQ ID NO: 11 or SEQ ID NO: 12 (the complementary sequence of SEQ ID NO: 11), wherein the cis-regulatory element improves endothelial cell specificity of a promoter compared to a promoter without the cis-regulatory element. The cis-regulatory element can further comprise an additional nucleotide sequence at least 60%, 70%, 80%, 90%, 95%, 96%, 97%, 98%, 99%, or 100% identical to SEQ ID NO: 13 or SEQ ID NO: 14 (the complementary sequence of SEQ ID NO: 13).

In another embodiment, a cis-regulatory element for the disclosure comprises a nucleotide sequence at least 60%, 70%, 80%, 90%, 95%, 96%, 97%, 98%, 99%, or 100% identical to SEQ ID NO: 13 or SEQ ID NO: 14 (the complementary sequence of SEQ ID NO: 13), wherein the cis-regulatory element improves endothelial cell specificity of a promoter compared to a promoter without the cis-regulatory element. The cis-regulatory element can further comprise an additional nucleotide sequence at least 60%, 70%, 80%, 90%, 95%, 96%, 97%, 98%, 99%, or 100% identical to SEQ ID NO: 11 or SEQ ID NO: 12 (the complementary sequence of SEQ ID NO: 11).

In other embodiments, a cis-regulatory element for the disclosure comprises a nucleotide sequence at least 60%, 70%, 80%, 90%, 95%, 96%, 97%, 98%, 99%, or 100% identical to SEQ ID NO: 15 or SEQ ID NO: 16 (the complementary sequence of SEQ ID NO: 15), wherein the cis-regulatory element improves endothelial cell specificity of a promoter compared to a promoter without the cis-regulatory element. In yet other embodiments, a cis-regulatory element for the nucleic acid construct comprises SEQ ID NO: 15 or SEQ ID NO: 16 or any fragments, variants, derivatives, or analogs thereof, wherein the fragments, variants, derivatives, or analogs improve endothelial cell specificity of a promoter compared to a promoter without the cis-regulatory element.

In some embodiments, a cis-regulatory element for the disclosure comprises a nucleotide sequence at least 60%, 70%, 80%, 90%, 95%, 96%, 97%, 98%, 99%, or 100% identical to SEQ ID NO: 20 or SEQ ID NO: 21, wherein the cis-regulatory element improves endothelial cell specificity of a promoter compared to a promoter without the cis-regulatory element. In yet other embodiments, a cis-regulatory element for the nucleic acid construct comprises SEQ ID NO: 20 or SEQ ID NO: 21 or any fragments, variants, derivatives, or analogs thereof, wherein the fragments, variants, derivatives, or analogs improve endothelial cell specificity of a promoter compared to a promoter without the cis-regulatory element.

In other embodiments, a cis-regulatory element for the disclosure comprises a nucleotide sequence at least 60%, 70%, 80%, 90%, 95%, 96%, 97%, 98%, 99%, or 100% identical to SEQ ID NO: 22 or SEQ ID NO: 23, wherein the cis-regulatory element improves endothelial cell specificity of a promoter compared to a promoter without the cis-regulatory element. In yet other embodiments, a cis-regulatory element for the nucleic acid construct comprises SEQ ID NO: 22 or SEQ ID NO: 23 or any fragments, variants, derivatives, or analogs thereof, wherein the fragments, variants, derivatives, or analogs improve endothelial cell specificity of a promoter compared to a promoter without the cis-regulatory element.

Table 3 shows various cis-regulatory element sequences useful for the disclosure.

TABLE 3
Endothelial Cell-Specific Cis-regulatory Elements and Promoters

| SEQ ID NOs | Sequences |
|---|---|
| SEQ ID NO: | ctggagggtg actttgcttc tggagccagt acttcatact tttcatt |
| 11 | |
| SEQ ID NO: | aatgaaaagt atgaagtact ggctccagaa gcaaagtcac cctccag |
| 12 | |
| SEQ ID NO: | gtacttcata cttttcattc caatggggtg actttgcttc tgga |
| 13 | |
| SEQ ID NO: | tccagaagca aagtcacccc attggaatga aaagtatgaa gtac |
| 14 | |
| SEQ ID NO: | 3X element |
| 15 | ctccagaagcaaagtcaccccattggaatgaaaagtatgaagtacaatgaaaagtatgaagt |
| | actggctccagaagcaaagtcaccctccagaagcaaagtcaccccattggaatgaaaagtat |
| | gaagtac |
| SEQ ID NO: | 3x element (Complementary Sequence of SEQ ID NO: 15) |
| 16 | gtacttcatacttttcattccaatggggtgactttgcttctggagggtgactttgcttctgg |
| | agccagtacttcatacttttcattgtacttcatacttttcattccaatggggtgactttgct |
| | tctggag |
| SEQ ID NO: | PPE-1 Promoter |
| 17 | gtacgtgtacttctgatcggcgatactagggagataaggatgtgcctgacaaaaccacattg |
| | ttgttgttatcattattatttagttttccttccttgctaactcctgacggaatctttctcac |
| | ctcaaatgcgaagtactttagtttagaaaagacttggtggaaggggtggtggtggaaaagta |
| | gggtgatcttccaaactaatctggttccccgcccgccccagtagctgggattcaagagcgaa |
| | gagtggggatcgtccccttgtttgatcagaaagacataaaaggaaaatcaagtgaacaatga |
| | tcagccccacctccaccccacccccctgcgcgcgcacaatacaatctatttaattgtacttc |
| | atacttttcattccaatggggtgactttgcttctggagaaactcttgattcttgaactctgg |
| | ggctggcagctagcaaaaggggaagcgggctgctgctctctgcaggttctgcagcggtctct |
| | gtctagtgggtgttttctttttcttagccctgcccctggattgtcagacggcgggcgtctgc |
| | ctctgaagttagccgtgatttcctctagagccgggtcttatctctggctgcacgttgcctgt |
| | gggtgactaatcacacaataacattgtttagggctggaatgaagtcagagctgtttaccccc |
| | actctataggggttcaatataaaaaggcggcggagaactgtccgagtcagaagcgttcctgc |
| | accggcgctgagagcctgacccggtctgctccgctgtccttgcgcgctgcctcccggctgcc |
| | cgcgacgctttcgccccagtggaagggccacttgctgcggccgc |
| SEQ ID NO: | PPE-1-3X promoter |
| 18 | gtacgtgtacttctgatcggcgatactagggagataaggatgtgcctgacaaaaccacattg |
| | ttgttgttatcattattatttagttttccttccttgctaactcctgacggaatctttctcac |
| | ctcaaatgcgaagtactttagtttagaaaagacttggtggaaggggtggtggtggaaaagta |
| | gggtgatcttccaaactaatctggttccccgcccgccccagtagctgggattcaagagcgaa |
| | gagtggggatcgtccccttgtttgatcagaaagacataaaaggaaaatcaagtgaacaatga |
| | tcagccccacctccaccccacccccctgcgcgcgcacaatacaatctatttaattgtacttc |
| | atacttttcattccaatggggtgactttgcttctggagaaactcttgattcttgaactctgg |
| | ggctggcagctagcctccagaagcaaagtcaccccattggaatgaaaagtatgaagtacaat |
| | gaaaagtatgaagtactggctccagaagcaaagtcaccctccagaagcaaagtcaccccatt |
| | ggaatgaaaagtatgaagtacgctagcaaaaggggaagcgggctgctgctctctgcaggttc |
| | tgcagcggtctctgtctagtgggtgttttctttttcttagccctgcccctggattgtcagac |
| | ggcgggcgtctgcctctgaagttagccgtgatttcctctagagccgggtcttatctctggct |
| | gcacgttgcctgtgggtgactaatcacacaataacattgtttagggctggaatgaagtcaga |
| | gctgtttacccccactctataggggttcaatataaaaaggcggcggagaactgtccgagtca |
| | gaagcgttcctgcaccggcgctgagagcctgacccggtctgctccgctgtccttgcgcgctg |
| | cctcccggctgcccgcgacgctttcgccccagtggaagggccacttgctgcggccgc |
| SEQ ID NO: | ggtgactttg cttctggag |
| 20 | |
| SEQ ID NO: | ctccagaagcaaagtcacc |
| 21 | |
| SEQ ID NO: | gtacttcata cttttcatt |
| 22 | |
| SEQ ID NO: | aatgaaaagtatgaagtac |
| 23 | |
| SEQ ID NO: | Hypoxia Response element |
| 24 | gcacgt |

A cis-regulatory element for the present disclosure can be linked to a promoter upstream or downstream of the promoter or inserted between the two nucleotides in the promoter. The endothelial cell-specific promoter for the present disclosure can utilize any promoters known in the art. For example, suitable promoters which can be utilized for the present disclosure include the endothelial-specific promoters: preproendothelin-1 (PPE-1 promoter), US 2010/0282634, published Nov. 11, 2010; and WO 2011/083464, published Jul. 14, 2011); the PPE-1-3X promoter (U.S. Pat. Nos. 7,579,327, 8,071,740, 8,039,261, US2010/0282634, US 2007/0286845, WO 2011/083464, and WO2011/083466); the TIE-1 (S79347, S79346) and the TIE-2 (U53603) promoters [Sato T N, Proc Natl Acad Sci U S A 1993 Oct. 15; 90(20):9355-8], the Endoglin promoter [Y11653; Rius C, Blood 1998 Dec. 15; 92(12):4677-90], the von Willerbrand factor [AF152417; Collins C J Proc Natl Acad Sci U S A 1987 July; 84(13):4393-7], the KDR/flk-1 promoter [X89777, X89776; Ronicke V, Circ Res 1996 August; 79(2):277-85],The FLT-1 promoter [D64016 AJ224863; Morishita K,: J Biol Chem 1995 Nov. 17; 270(46):27948-53], the Egr-1 promoter [AJ245926; Sukhatme V P, Oncogene Res 1987 September-October; 1(4):343-55], the E-selectin promoter [Y12462;Collins T J Biol Chem 1991 Feb. 5; 266(4):2466-73], The endothelial adhesion molecules promoters: ICAM-1 [X84737; Horley K J EMBO J 1989 October; 8(10):2889-96], VCAM-1 [M92431; Iademarco M F , J Biol Chem 1992 Aug. 15; 267(23): 16323-9], PECAM-1 [AJ313330 X96849; CD31, Newman P J, Science 1990 Mar. 9; 247(4947): 1219-22], the vascular smooth-muscle-specific elements: CArG box X53154 and aortic carboxypeptidase-like protein (ACLP) promoter [AF332596;Layne M D, Circ Res. 2002; 90: 728-736] and Aortic Preferentially Expressed Gene-1 [Yen-Hsu Chen J. Biol. Chem, Vol. 276, Issue 50, 47658-47663, Dec. 14, 2001], all of which are incorporated herein by reference in their entireties.

In one embodiment, a promoter linked to the endothelial cell-specific element comprises a nucleotide sequence at least 60%, 70%, 80%, 90%, 95%, 96%, 97%, 98%, 99%, or 100% of SEQ ID NO: 17, wherein the promoter linked to the element induces endothelial cell-specificity to the gene operably linked to the promoter. In another embodiment, a promoter linked to the endothelial cell-specific element comprises a fragment, a variant, a derivative, or an analog of a wild-type PPE-1 promoter, wherein said fragment, variant, derivative, or analog thereof induces endothelial cell-specificity to the gene operably linked to the promoter. In one example, the endothelial cell-specific element can be inserted between nucleotide residues 442 and 449 corresponding to SEQ ID NO: 17.

In further embodiments, an endothelial cell-specific promoter comprises a hypoxia responsive element. A hypoxia responsive element (HRE) is located on the antisense strand of the endothelin-1 promoter. This element is a hypoxia-inducible factor-1 binding site that is required for positive regulation of the endothelin-1 promoter (of the human, rat and murine gene) by hypoxia. Hypoxia is a potent signal, inducing the expression of several genes including erythropoietin (Epo), VEGF, and various glycolytic enzymes. The core sequence (8 base pairs) is conserved in all genes that respond to hypoxic conditions and the flanking regions are different from other genes. The ET-I hypoxia responsive element is located between the GAT A-2 and the AP-1 binding sites. In one example, a hypoxia response element comprises SEQ ID NO: 24, a fragment, a variant, a derivative, or an analog thereof.

In other embodiments, an endothelial cell-specific promoter useful for the disclosure comprises, consists essentially of, or consists of a nucleotide sequence at least 60%, 70%, 80%, 90%, 95%, 96%, 97%, 98%, 99%, or 100% of SEQ ID NO: 18, wherein the promoter linked to the cis-regulatory element induces endothelial cell-specificity to the gene operably linked to the promoter. In another embodiment, an endothelial cell-specific promoter comprises a fragment, a variant, a derivative, or an analog of SEQ ID NO: 18, wherein said fragment, variant, derivative, or analog thereof induces endothelial cell-specificity to the gene operably linked to the promoter.

Additional variations of the endothelial cell-specific promoters can be found at WO2011/083464, WO2011/083466, and WO2012/052423, which are incorporated herein by reference in their entireties.

The present disclosure also provides a novel promoter sequence comprising a nucleotide sequence SEQ ID NO: 17. In one example, the promoter further comprises an endothelial cell-specific cis-regulatory element. In one example, the endothelial cell-specific cis-regulatory element comprises SEQ ID NO: 11, SEQ ID NO: 12, SEQ ID NO: 13, SEQ ID NO: 14, SEQ ID NO: 15, SEQ ID NO: 16, SEQ ID NO: 20, SEQ ID NO: 21, SEQ ID NO: 22, SEQ ID NO: 23, SEQ ID NO: 24 or any fragments, derivatives, variants, or analogs thereof, wherein the fragments, derivatives, variants, or analogs thereof improve endothelial cell-specificity of the promoter compared to a promoter without the cis-regulatory element. In another example, the promoter comprises a nucleotide sequence of SEQ ID NO: 18. The disclosure includes a nucleic acid construct comprising the novel promoter and a heterologous nucleotide sequence. In one embodiment, the heterologous nucleic acid sequence comprises a nucleotide sequence encoding a Fas-chimera protein described herein. In another embodiment, the heterologous nucleotide sequence comprises an adenovirus sequence.

C. Vector

The present disclosure also provides a vector comprising the nucleic acid construct, which comprises a Fas-chimera gene operably linked to an endothelial cell-specific promoter. For the purposes of this disclosure, numerous vector systems can be employed. For example, various viral gene delivery systems that can be used in the practice of this aspect of the disclosure include, but are not limited to, an adenoviral vector, an alphavirus vector, an enterovirus vector, a pestivirus vector, a lentiviral vector, a baculoviral vector, a herpesvirus vector, an Epstein Barr viral vector, a papovaviral vector, a poxvirus vector, a vaccinia viral vector, an adeno-associated viral vector and a herpes simplex viral vector.

In another embodiment, a vector comprising a Fas-chimera gene operably linked to an endothelial cell-specific promoter is an adenovirus. For example, the adenovirus can be any one or more of human adenovirus species A (serotypes 12, 18, and 31), B (serotpyes 3, 7, 11, 14, 16, 21, 34, 35, 50, and 55), C (serotypes 1, 2, 5, 6, and 57), D (8, 9, 10, 13, 15, 17, 19, 20, 22-30, 32, 33, 36-39, 42-49, 51, 53, 54, and 56), E (serotype 4), F (serotype 40 and 41), or G (serotype 52). In a particular embodiment, the adenovirus for the disclosure is human adenovirus serotype 5. In some embodiments, the adenovirus useful for gene therapy is a recombinant non-replicating adenovirus, which does not contain an E1 region and an E3 region.

In a particular aspect, the vector is an Ad5-PPE-1-3X-Fas-c vector. In a more particular aspect, the vector is an Ad5-PPE-1-3X-Fas-c vector that comprises, consists essentially of, or consists of SEQ ID NO: 19. In another embodiment, the adenovirus vector is an isolated virus having European Collection of Cell Cultures (ECACC) Accession Number 13021201.

### IV. Treatment Further Comprising One or More Chemotherapeutic Agents

In some aspects, the methods of the present disclosure further comprise administering to the subject one or more chemotherapeutic agents.

One or more chemotherapeutic agents that can be administered using the methods of the present disclosure include, but are not limited to, Acivicin; Aclarubicin; Acodazole Hydrochloride; Acronine; Adriamycin; Adozelesin; Aldesleukin; Alimta; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bevacizumab, Bizelesin; Bleomycin Sulfate; Brequinar Sodium; Bropirimine; Busulfan; Cactinomycin; Calusterone; Caracemide; Carbetimer; Carboplatin; Carmustine (BiCNU); Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Gliadel® wafer; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine (CCNU); Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; pazotinib; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sorafinib; Sparfosate Sodium; Sparsomycin; Spirogermanium Hydrochloride; Spiromustine; Spiroplatin; Streptonigrin; Streptozocin; Sulofenur; Sunitinib; Talisomycin; Taxol; Tecogalan Sodium; Tegafur; Teloxantrone Hydrochloride; Temoporfin; Temozolomide; Teniposide; Teroxirone; Testolactone; Thiamiprine; Thioguanine; Thiotepa; Tiazofuirin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; or Zorubicin Hydrochloride. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc.

In some aspects of the disclosure, the one or more chemotherapeutic agents are selected from the group consisting of altretamine, raltritrexed, topotecan, paclitaxel, docetaxel, cisplatin, carboplatin, oxaliplatin, liposomal doxorubicin, gemcitabine, cyclophosphamide, vinorelbine, ifosfamide, etoposide, altretamine, capecitabine, irinotecan, melphalan, pemetrexed, bevacizumab, and albumin bound paclitaxel.

In some aspects, the subject has had up to three, up to two, or up to one previous line of chemotherapy. In other aspects, the subject has not had more than 3 prior lines of chemotherapy for recurrent cancer.

An effective dose of the chemotherapeutic agents is available in the art.

In some aspects, the one or more chemotherapeutic agents are repeatedly administered. In particular aspects, the one or more chemotherapeutic agents are repeatedly administered once in about 7 days, once in about 2 weeks, once in about 3 weeks, once in about 4 weeks, once in about 2 months, once in about 3 months, once in about 4 months, once in about 5 months, or once in about 6 months.

### V. Pharmaceutical Compositions

Also provided in the disclosure is a pharmaceutical composition comprising a vector expressing a Fas-chimera protein used in the methods of the disclosure. The pharmaceutical composition can be formulated for administration to mammals, including humans. The pharmaceutical compositions used in the methods of this disclosure comprise pharmaceutically acceptable carriers, including, e.g., ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. In one embodiment, the composition is formulated by adding saline.

The compositions of the present disclosure can be administered by any suitable method, e.g., parenterally (e.g., includes subcutaneous, intravenous, intramuscular, intra-articular, intra-synovial, intrasternal, intrathecal, intrahepatic, intralesional and intracranial injection or infusion techniques), intraventricularly, orally, by inhalation spray, topically, rectally, nasally, buccally, vaginally or via an implanted reservoir. In one embodiment, the combination therapy is delivered systemically or locally. For systemic or local delivery, the pharmaceutical formulation can be administered using a mechanical device such as a needle, cannula, or surgical instruments.

Sterile injectable forms of the compositions used in the methods of this disclosure can be aqueous or oleaginous suspension. These suspensions can be formulated according to techniques known in the art using suitable dispersing or wetting agents and suspending agents. The sterile, injectable preparation can also be a sterile, injectable solution or suspension in a non-toxic parenterally acceptable diluent or solvent, for example as a suspension in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms can also be used for the purposes of formulation.

Parenteral formulations can be a single bolus dose, an infusion or a loading bolus dose followed with a maintenance dose. These compositions can be administered at specific fixed or variable intervals, e.g., once a day, or on an “as needed” basis.

Certain pharmaceutical compositions used in the methods of this disclosure can be orally administered in an acceptable dosage form including, e.g., capsules, tablets, aqueous suspensions or solutions. Certain pharmaceutical compositions also can be administered by nasal aerosol or inhalation. Such compositions can be prepared as solutions in saline, employing benzyl alcohol or other suitable preservatives, absorption promoters to enhance bioavailability, and/or other conventional solubilizing or dispersing agents.

### EXAMPLES

### Example 1

Evaluating the efficacy and safety of anti-PD-L1 antibody in Combination with VB-111 in Mice with metastatic Lewis Lung Carcinoma

Objective: The objective of this study was to explore the effectiveness and safety of a combined treatment with Ad5-PPE-1-3X-Fas-c and an anti PD-L1 monoclonal antibody in the metastatic Lewis Lung Carcinoma mouse model.

Materials: The following materials were used in this study:

Purified anti-mouse CD274 (B7-H1, PD-L1)
Catalog no: BLG-124328, BLG-124329 Hamster IgG
Concentration: 2.03 mg/ml
Supplied by: BIOLEGEND (by ENCO)
Physical state: Liquid
Lot No.: 8210782
Size: One vial of 25 mg and another vial of 5 mg
Vehicle: PBS
Preparation: Anti PD-L1 antibody was dissolved in PBS to a concentration of 1 mg/ml (200 μl per mouse gave a dose of 200 μg/mouse). Ad5-PPE-1-3X-Fas-c (VB-111) 10⁹ virus particles per mouse
Concentration: 1×10¹² virus particles (vp)/ml
Physical state: liquid
Storage conditions: ≤−65° C. , in cryogenic vials
Vehicle: Saline
Preparation: Vial was thawed on the day of treatment and mixed by inversions. VB-111 (1×10¹²/ml) was diluted by 100 fold. (By collecting 10 μl of VB-111 1×10¹²/ml and adding 990 μl saline in order to get a concentration of VB-111 1×10¹¹/ml ), mice had been administered 100 μl/mouse IV in order to achieve (1×10⁹ VP/mouse).

Ad5-PPE-1-3X-Fas-c (VB-111) 10¹¹ virus particles per mouse
Concentration: 1×10¹² virus particles (vp)/ml
Physical state: liquid
Storage conditions: ≤−65° C. , in cryogenic vials
Vehicle: Saline
Preparation: Vial was thawed on the day of treatment and mixed by inversions. VB-111 (1×10¹²/ml ) was administered at 100 μl/mouse in order to achieve 1×10¹¹ virus particles/mouse.

Negative control/vehicle
Name: Saline
Supplied by: LIFE
Physical state: Liquid
Lot number: G172446
Size: Each vial contains 5 m1
Storage conditions: room temperature

### Animals

Male C57BL/6 mice, 12-14 weeks old, were used in this study. Care of mice and handling procedures were in accordance with the Guide for the Care and Use of Laboratory Animals printed by the Institute of Laboratory Animals, National Academy Press (Washington, D.C.).

### Methods:

Male C57BL/6 mice (12-14 weeks) were injected in the left footpad with 5×10^{5 D}122 cells in 50 μl with the exception of group F (healthy untreated group).

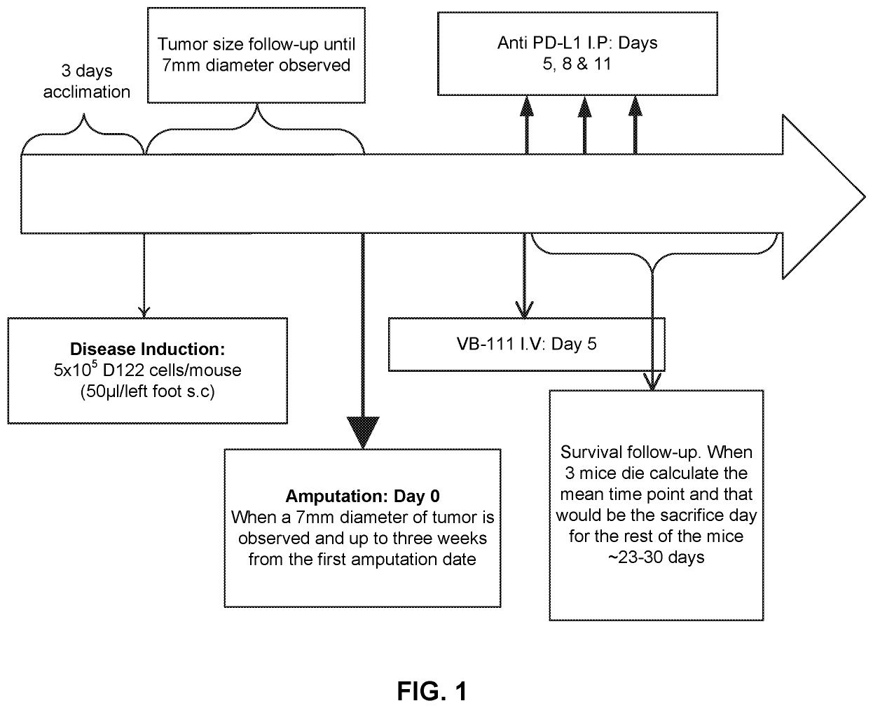

Mice were monitored for tumor diameter every 5 days. When the tumor diameter reached 5 mm, mice were followed daily until the tumor diameter reached 7 mm. The day in which the tumor diameter reached 7 mm was determined as day 0 and the primary tumor was removed by amputation. Upon amputation, mice were randomly divided to the different treatment groups (FIG. 1 and Table 4). Treatment began 5 days following amputation. Each animal received:
A. Saline IV 100 μl;
B. VB-111 (1×10¹¹ VP/mouse, IV in 100 μl) treatment once, on day 5;
C. VB-111 (1×10⁹ VP/mouse, IV in 100 μl) treatment once, on day 5;
D. VB-111 (1×10⁹ VP/mouse, IV in 100 μl) treatment once, on day 5 and anti PD-L1 antibody (200 μg/mouse, IP) treatment, once every 3 days on days 5, 8 and 11;
E. Anti PD-L1 antibody (200 μg/mouse, IP) treatment, once every 3 days on days 5, 8 and 11; or
F. No-Treatment (Healthy group).

The mean number of days passed from amputation to death for first three consecutive deaths determined the number of days for sacrifice from amputation for the remaining animals.

The treatment cohorts are summarized below in Table 4.

TABLE 4
Test groups and dose levels

| | | | Number |
|---|---|---|---|
| Cohort | Disease induction | Treatment | (n) |
| A | Metastases in the lung | Saline IV | 25 |
| B | were developed | VB-111, lx10¹¹, IV once on day | 19 |
| | following injection of | 5 | |
| C | D122 5x10⁵ cells/Mouse | VB-111, 1x10⁹, IV once on day | 19 |
| | in the left footpad and | 5 | |
| D | resection of the primary | VB-111, lx10⁹, IV once on day | 17 |
| | tumor when the primary | 5, and anti-PD-Ll antibody 200 | |
| | tumor reaches a | μg/mouse, IPR, 3 times, once | |
| | diameter of 7mm (Day0) | every 3 days 5, 8, 11 | |
| E | | anti-PD-Ll antibody 200 | 19 |
| | | μg/mouse, IPR, 3 times, once | |
| | | every 3 days 5, 8, 11 | |
| F | None | None | 5 |

Efficacy evaluation: Lung weight was recorded, photographed and collected in formaldehyde 4%. Tumor burden was calculated by subtraction of normal lung weight (0.1526 g). Each parameter tested was analyzed by T test.

### Results:

### Body Weight

There was no significant difference in mean body weight between all treated groups. A significant difference was observed between the saline group and the healthy group (p<0.05).

### Lung Weight

Mean lung weight of the negative control, saline treated group, was increased by 6 fold over the healthy group.

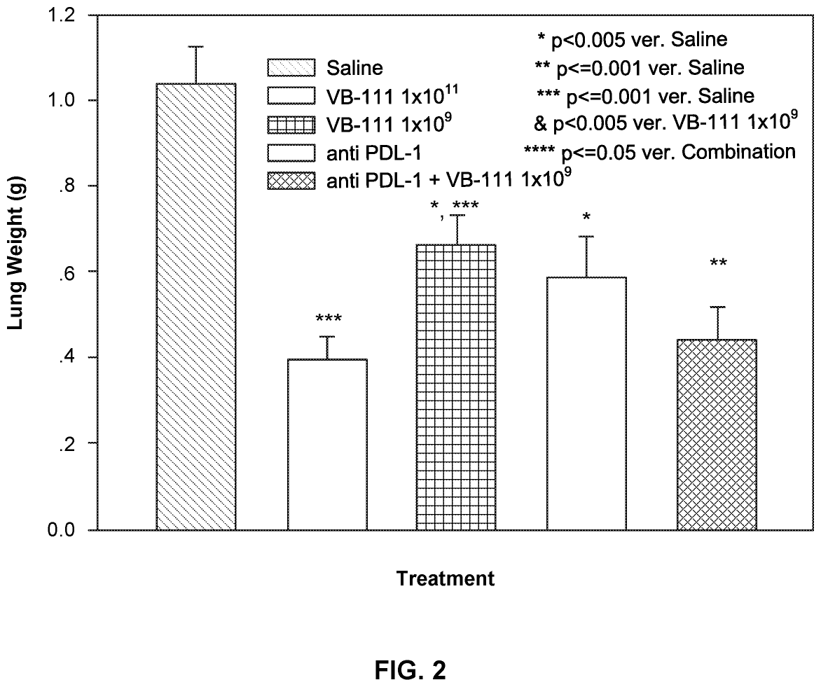

Treatment with VB-111 1×10¹¹ VP/mouse demonstrated the most robust effect, significantly (p≤0.001) reducing mean lung weight by 62%. VB-111 at a lower dose of 1×10⁹ VP/mouse significantly reduced lung weight by 36% (p<0.005), similar to the 44% reduction observed following anti PD-L1 treatment (p≤0.001). However, combined treatment of VB-111 1×10⁹ VP/mouse with anti PD-L1 resulted in a reduction rate in mean lung weight of 58% (p≤0.001), which was similar to the rate observed following treatment with the high dose VB-111 at 1×10¹¹ VP/mouse. Combined treatment of VB-111 at 1×10⁹ VP/mouse and anti PD-L1 was more effective significantly than monotherapy with VB-111 at 1×10⁹ VP/mouse (p<0.05) (FIG. 2).

### Tumor Burden

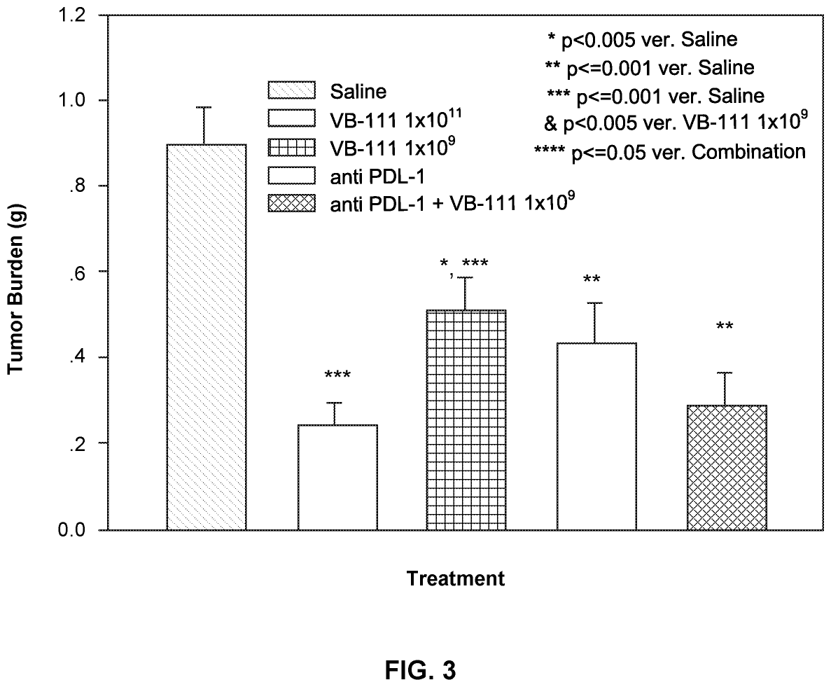

Treatment with VB-111 at 1×10¹¹ VP/mouse demonstrated the most robust effect, significantly (p≤0.001) reducing mean tumor burden by 72%. VB-111 at a lower dose of 1×10⁹ VP/mouse significantly reduced mean tumor burden by 42% (p<0.005), and a reduction of 51% was observed following anti PD-L1 treatment (p≤0.001). However, combined treatment of VB-111 at 1×10⁹ VP/mouse with anti PD-L1 resulted in a profound reduction of 67% (p≤0.001), similar to the reduction observed following treatment with the high dose of VB-111 at 1×10¹¹ VP/mouse. Combined treatment of VB-111 at 1×10⁹ VP/mouse and anti PD-L1 was significantly more effective than monotherapy with VB-111 at 1×10⁹ VP/mouse (p<0.05S) (FIG. 3).

### Conclusion

In this study, treatment with high dose of VB-111 (1×10¹¹ VP/mouse) significantly reduced mean lung weight and tumor burden in LLC mouse model. Treatment with a lower dose of VB-111 (1×10⁹ VP/mouse) in combination with anti PD-L1 showed superiority in reducing mean tumor lung burden over monotherapy with anti PD-L1 or VB-111 at 1×10⁹ VP/mouse and similar effect to monotherapy with the high dose of VB-111 at 1×10¹¹ VP/mouse.

### Example 2

Evaluating the Efficacy and Safety of Anti-PD-L1 Antibody in Combination with VB-111 in Mice with B16F10 Melanoma Tumors

Objective: The objective of this study was to explore the effectiveness and safety of a combined treatment with Ad5-PPE-1-3X-Fas-c and an anti PD-L1 monoclonal antibody in a melanoma tumor mouse model.

Materials: The following materials were used in this study:

Purified anti-mouse CD274 (B7-H1, PD-L1)
Catalog no: BLG-124329, Hamster IgG
Concentration: 2.0 mg/ml
Supplied by: BIOLEGEND (by ENCO)
Physical state: Liquid
Lot No.: B214210
Size: One vial of 25 mg
Vehicle: PBS
Preparation: Anti PD-L1 antibody was dissolved in PBS to a concentration of 1 mg/ml (200 μl per mouse gave a dose of 200 μg/mouse).

Ad5-PPE-1-3X-Fas-c (VB-111) 10¹¹ virus particles per mouse
Concentration: 1×10¹² virus particles (vp)/ml
Physical state: liquid
Storage conditions: ≤−65° C. , in cryogenic vials
Vehicle: Saline
Preparation: Vial was thawed on the day of treatment and mixed by inversions. VB-111 (1×10¹²/ml ) was administered 100 μl/mouse in order to achieve 1×10¹¹ VP/mouse.

Negative control/vehicle
Name: Saline
Supplied by: LIFE
Physical state: Liquid
Lot number: G172446
Size: Each vial contains 5 ml
Storage conditions: room temperature s

Animals

Male C57BL/6 mice, 12-14 weeks old, were used in this study. The animals were 12-14 weeks of age at the initiation of the study. Care of mice and handling procedures were in accordance with the Guide for the Care and Use of Laboratory Animals printed by the Institute of Laboratory Animals, National Academy Press (Washington, D.C.).

### Methods:

Male C57BL/6 mice (12-14 weeks) were injected with 2×10⁵ B16F10 cells in 50 μl of PBS+50 μl MATRIGEL to the left flank subcutaneously.

Mice were monitored for tumor volume three to six times per week. Treatment began on day 9 (“assignment day”), when animal developed tumors that reach approximately 100 mm³ Mice were randomly assigned to the different groups based on tumor volume and body weight (at that time point mice that did not show any measurable tumor or mice bearing a fluid tumor were excluded). Data on mice body weight was recorded 3 times a week and clinical signs were recorded 3-6 times a week.

Each animal received:
A. Saline IV;
B. VB-111 (1×10¹¹ VP/mouse, IV in 100 μl) treatment once, on day 9;
C. Anti PD-L1 antibody (200 μg/mouse, IP) treatment, once every 2-3 days on days 9, 12, and 14; or
D. VB-111 (1×10¹¹ VP/mouse, IV in 100 μl) treatment once, on day 9 and anti-PD-L1 antibody (200 μg/mouse, IP) treatment, once every 2-3 days on days 9, 12, and 14.
The treatment cohorts are summarized below in Table 5.

TABLE 5
Test groups and dose levels

| Co- | Disease induction | | Number |
|---|---|---|---|
| hort | (Day 0) | Treatment | (n) |
| A | B16F10 50 μ1 4 x 10⁶ | Saline 100 μ1 IV | 12 |
| B | cells/ml in PBS mixed | VB-111, lx10¹¹ VPs | 12 |
| | with equal volume of | (100 μ1 of VB-111 lx10¹² | |
| | MATRIGEL, total 100 | VPs/ml) IV once upon group | |
| | μ1/mouse injected to | assignment | |
| C | the left flank | anti-PD-Ll antibody 200 | 11 |
| | subcutaneously. | μg/mouse in 200 μ1 PBS, IP 3 | |
| | | times, every 2-3 days starting on | |
| | | group assignment day | |
| D | | VB-111, 1x10¹¹, IV once upon | 11 |
| | | group assignment and anti-PD- | |
| | | Ll antibody 200 μg/mouse, IP, 3 | |
| | | times every 2-3 days starting on | |
| | | group assignment day | |

Efficacy evaluation: Individual tumor volume was recorded, photographed and collected in formaldehyde 4%.

### Results:

### Body Weight

There was no significant difference in mean body weight between all treated groups.

Tumor Volume (mm³)

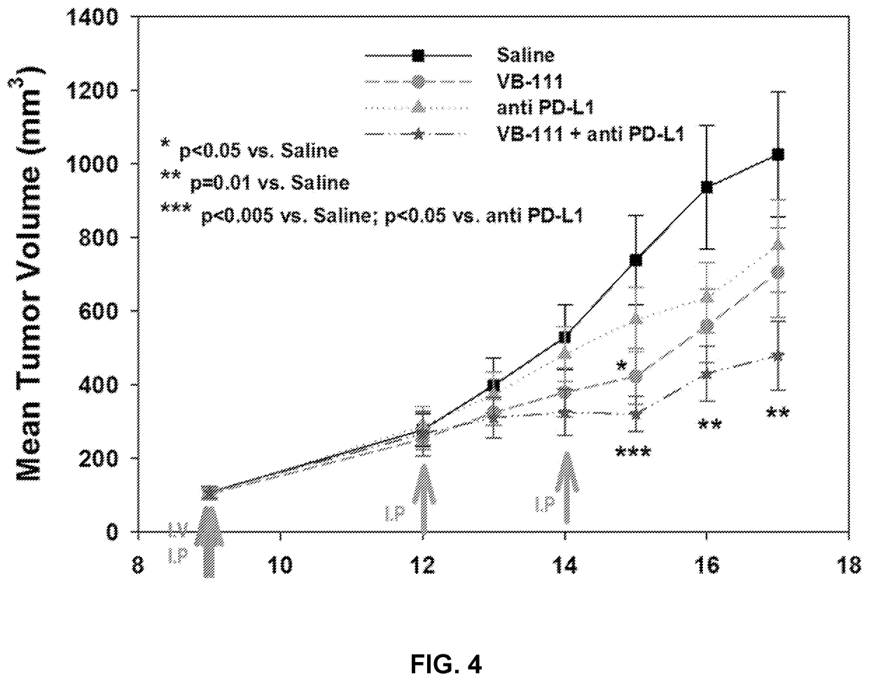

Treatment with VB-111 1×10¹¹ VP/mouse alone or with anti PD-L1 alone moderately reduced tumor volume; however the most robust effect was following combination therapy of VB-111 and anti PD-L1. The combined treatments significantly reduced mean tumor volume from day 15 until sacrifice. The value of combined treatment was most pronounced up to day 17 (FIG. 4).

### Conclusion

Treatment with VB-111 (1×10¹¹ VP/mouse) in combination with anti PD-L1 showed superiority in reducing mean tumor volume over monotherapy with anti PD-L1 or VB-111 at 1×10¹¹ VP/mouse.

### Example 3

A Phase I/II Randomized, Open-Label, Multicenter Study of VB-111 Combined with Nivolumab in Patients with Previously Treated Advanced or Metastatic Non-Squamous Cell Non-Small Cell Lung Cancer (NSCLC)

### Study Design and Treatment Plan

This open-label study aims to evaluate the safety and efficacy of VB-111, intravenously (IV) administered every two months, in combination with Nivolumab, infused as a standard of care at 3 mg/kg every two weeks, compared to Nivolumab alone, in patients with advanced or metastatic non-squamous cell NSCLC. The study will begin with a single-arm, multi-center, dose escalation, phase I component in which this combination will be given to up to 12 patients using the 3+3 dose escalation model, and if successful, further enrollment into a randomized Phase II will proceed, as detailed below.

Phase I Component: Dose Level 1 (Cohort 1): VB-111 3×10¹² viral particles (VPs)+Nivolumab 3 mg/kg.

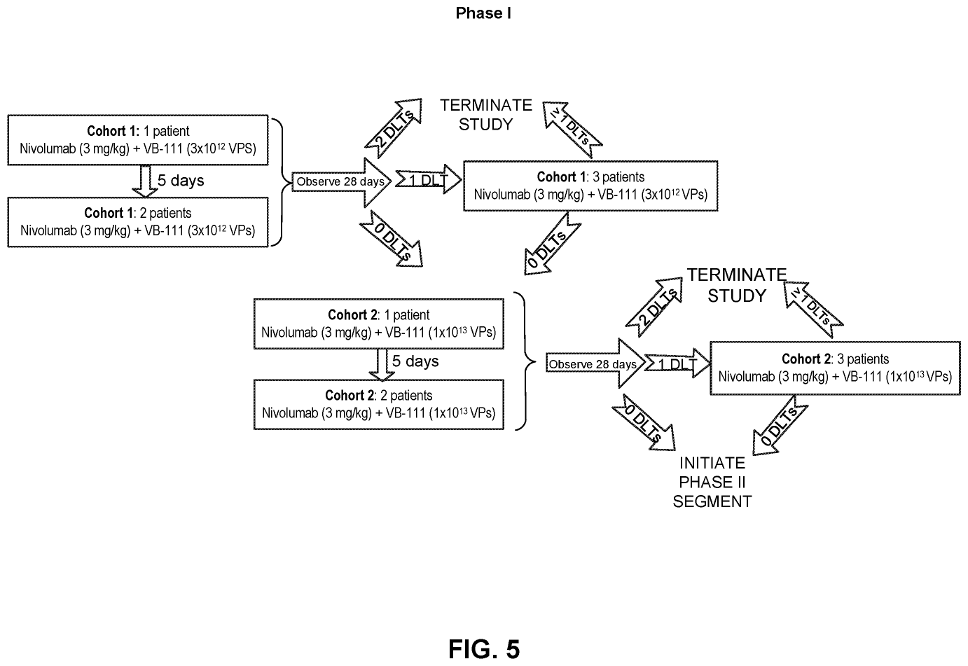

The Phase I components are depicted in FIG. 5. At least 3 patients will be treated with IV infusion of Nivolumab (3 mg/kg), followed by IV infusion of VB-111 (3×10¹² viral particles (VPs)) and observed for the occurrence of dose-limiting toxicities (DLTs) for 28 days. At first, only one patient will be enrolled and start treatment while the two additional patients will be enrolled at least 5 days after patient 1 start of treatment day. If no DLTs are recorded in the first set of 3 patients during the 28 days period, then, Cohort 2 will be opened for recruitment. However, if two DLTs are recorded in the first set of 3 patients, the trial will be terminated. If only one DLT is observed, three additional patients will be administered with the same dose level 1 and DLTs will be assessed for up to 28 days. If one DLT is observed in this second set of patients (i.e., 2/6 patients experience a DLT), the trial will be terminated. Otherwise, Cohort 2 will be opened for recruitment.

Phase I Component: Dose Level 2 (Cohort 2): VB-111 1×10¹³ VPs+Nivolumab 3 mg/kg.

Initiation of Cohort 2 enrollment will only be authorized after all patients of Cohort 1 have completed a 28-day observation period and fewer than 2 DLTs are reported. See FIG. 5. In this Cohort 2, at least 3 patients will be treated with IV infusion of Nivolumab (3 mg/kg), followed by IV infusion of VB-111 (1×10¹³ VPs) and observed for the occurrence of DLTs for 28 days. Cohort 2 enrollment scheduling will be similar to that of Cohort 1: at first, only one patient will be enrolled and start treatment while the two additional patients will be enrolled at least 5 days after patient 1 start of treatment day. If no DLTs are recorded in the first set of 3 patients during the 28 days period, than, this dose will be determined as safe for the combination treatment and used as the recommended phase II dose (RP2D). However, if two DLTs are recorded in the first set of 3 patients, the trial will be terminated. If only one DLT is observed, three additional patients will be administered with the same dose level 2 and DLTs will be assessed for up to 28 days. If one DLT is observed in this second set of patients (i.e., 2/6 patients experience a DLT), the trial will be terminated. Otherwise, enrollment into Phase II component of this study will be authorized.

At that time, intra-patient dose escalation is allowed: i.e., patients treated at Dose Level 1 may be escalated to receive subsequent treatment at Dose Level 2. All of the patients entering Phase I will be evaluated for efficacy in Phase II analysis of the trial.

Definition of DLT: Any drug-related (either VB-111 or Nivolumab) grade >3 toxicity occurring during the first 28 days of treatment, excluding the following:
Grade≥3 hepatic or hematologic toxicities.
Grade≥3 nausea or vomiting that can be controlled medically (If nausea and/or vomiting cannot be controlled medically and occurs during the 28 days observation period , it will be considered a DLT).
Grade≥3 hypokalemia, hyponatremia, hypophosphatemia, hypomagnesemia, and hypocalcemia, if they can be easily corrected, are clinically asymptomatic, and not accompanied by medically significant complications (e.g., ECG changes).
Events of Grade 3-4 fever that occur within 24 hours post-dosing with VB-111 shall not be considered DLT if they respond to symptomatic therapy.
Events of Grade 3-4 which are commonly expected by Nivolumab treatment (for example Rash, thyroiditis, diarrhea, hepatitis, nephritis, pneumonitis) and considered by the Investigator as related to Nivolumab will not be considered as DLTs unless they are more severe in grade, extent, duration or onset time compared to Nivolumab monotherapy toxicity, as determined by the Investigator.

Patients who complete the 28 days observation period with no DLTs will continue to be treated with VB-111 on Day 1 of every fourth 14-day cycle (every 56±5 days) and Nivolumab on Day 1 of every 14-day cycle. On days when both drugs are administered, Nivolumab will be administered first. Those patients will continue all assessments and evaluations according to the schedule of events. Reductions in the VB-111 and/or the Nivolumab doses are not permitted (doses can be only delayed or discontinued as per treatment delay or discontinuation guidelines given in this protocol).

In case that DLTs are reported in this study, patients for whom the DLT is reported will discontinue study treatment. Efficacy FU should be performed for the DLT patients as for the rest of the patients who discontinued (continue FU for further anti-cancer treatments and CT scan collections). Further anti-cancer treatment for those patients will be given as per investigator's discretion.

Patients withdrawn during the 28-day observation period for any reason other than DLT will be replaced by a patient who will be treated with the same dose regimen. AEs occurring after the 28-day observation period will be recorded as AEs, even if they meet the DLT criteria. In the event of AEs meeting the DLT criteria after the 28-day observation period, the investigator will consult with the medical monitor for specific safety assessment guidelines.

### Phase II Component:

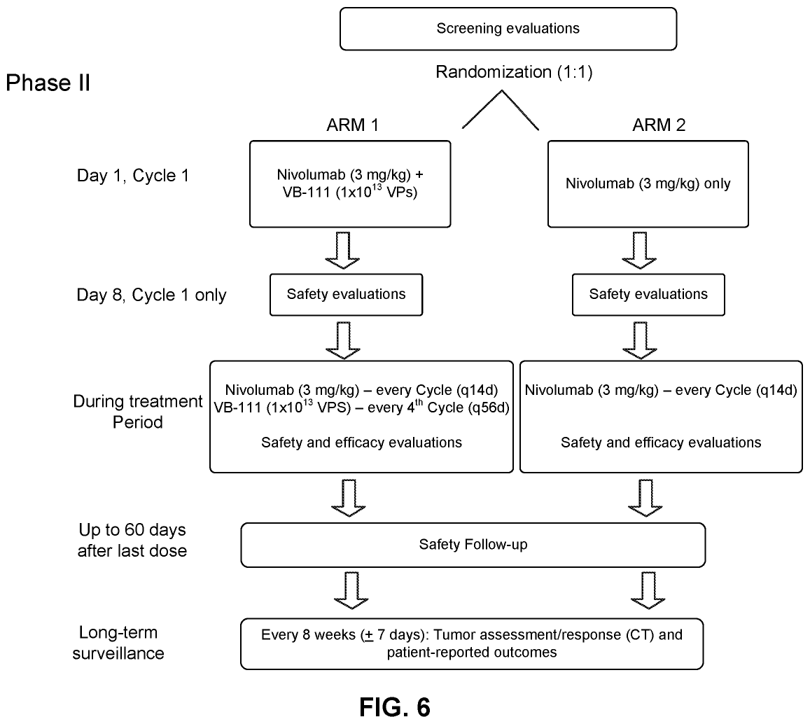

The Phase II component is depicted in FIG. 6. If fewer than 2 DLTs are reported in Cohort 2 patients, the phase II component will be initiated and opened for recruitment of new patients. In this part of the study, patients will be randomized into one of two treatment arms in a 1:1 ratio (investigative arm or control arm), using a centralized randomization procedure, to be treated with either:

Arm 1 (VB-111 Combined with Nivolumab):
VB-111 at a dose of lx10¹³ VPs (RP2D) given as an IV infusion on day 1 and every 4th 14-day cycle (every 56 days±5 days).
Nivolumab given as a standard of care at 3 mg/kg as an IV infusion on day 1 of each 14-day cycle.

Arm 2 (Nivolumab alone):
Nivolumab given as a standard of care at 3 mg/kg as an IV infusion on day 1 of each 14-day cycle.

In this study, one cycle length will be 14 days. First dose of study drug should be given within 48 hours after randomization. On days when both drugs are administered, Nivolumab will be administered first. Reductions in the VB-111 and/or the Nivolumab doses are not permitted (doses can be only delayed or discontinued as per treatment delay or discontinuation guidelines given in this protocol). Also, there will be no cross-over from control arm to combination arm. See FIG. 6.

Randomization will be stratified by the following stratification factors:
PD-L1 expression level <1% versus ≥1%
Smoking status: former light-smoker or non-smoker versus smoker
   Non-smoker, defined as patients who smoked ≤100 cigarettes in their lifetime;
   Former light smoker, defined as patients who smoked between >100 cigarettes AND ≤10 pack-years AND quit ≥1 year prior to enrollment;
Gender: male versus female.

### Study Treatment Duration

In both phases of the study (Phase I and II), treatment will continue until patients experience unacceptable treatment related toxicities, until confirmed disease progression (PD), as defined by irRECIST, or other reasons (e.g., withdrawn consent, investigator's discretion, disease progression that does not meet the discontinuation criteria as per investigator's discretion). Study treatment will be considered completed in patients discontinuing treatment due to confirmed PD. Discontinuation for any other reason will be considered incomplete treatment and will be recorded as “discontinued.”

Upon completion or discontinuation of study treatment, patients will be treated according to the physician's discretion. Every effort will be made to collect post-treatment scans (done as per standard of care), information on subsequent anti-cancer therapies and patient-reported outcome measures until death, withdrawal of consent or loss to follow up, at intervals defined by standard of care. For patients who discontinue study treatment for a reason other than PD, follow-up scans should be performed every 8 weeks (±7 days) until PD, withdrawal of consent, death, lost to follow up.

### Study Enrollment and Withdrawal

Deviations from any inclusion or exclusion criteria are not allowed because deviations can potentially jeopardize the scientific integrity of the study, regulatory acceptability, or subject safety. Therefore, adherence to the criteria as specified in the protocol is mandatory. Any questions regarding a subject's eligibility should be discussed with the Sponsor prior to enrollment.

Subject Inclusion Criteria. In order to be eligible to participate in this study, subjects must meet all of the following criteria:

Signed informed consent obtained prior to initiation of any study-specific procedures and treatment, as confirmation of the patient's awareness and willingness to comply with the study requirements.

### Female or Male Patients ≥18 Years of Age.

Patients with Stage IV or stage III histologically-documented non-squamous cell non-small cell lung cancer (NSCLC), or with recurrent disease and not a candidate for curative treatment but a candidate for second-line Nivolumab for advanced disease as standard of care.

Disease recurrence or progression during/after one prior platinum doublet-based chemotherapy regimen for advanced or metastatic NSCLC. Prior treatment for early disease (adjuvant or neo-adjuvant) can be counted as first-line treatment for stage IV, if disease recurs within 6 months after last platinum treatment.

Measurable disease by computed tomography (CT) per response evaluation criteria in solid tumors (RECIST) 1.1, performed within 28 days prior to first dose of study drug. Target lesions may be located in a previously irradiated field, if there is documented disease progression in that site.

Eastern Cooperative Oncology Group (ECOG) performance status <1.

Adequate renal, liver, and bone marrow function according to the following criteria:
Absolute neutrophil count ≥1500 cells/μl
Hemoglobin ≥9.0 g/dL
Platelets≥100,000 cells/μl
Total bilirubin ≤1.5-times upper limit of normal (ULN), except patients with Gilbert Syndrome who must have total bilirubin <3.0 mg/dL,
Aspartate aminotransferase (AST) and Alanine transaminase (ALT) ≤2.5 X ULN.
Serum creatinine level ≤1.5 ULN or creatinine clearance ≥40 ml/min for patients with creatinine levels above normal limits (creatinine clearance calculated by the Cockcroft-Gault formula)
Prothrombin Time (PT), Partial Thromboplastin Time (PTT) (in seconds) not to be prolonged beyond >20% of ULN, unless due to anticoagulant use;

Life expectancy of ≥12 weeks.

Radiotherapy including significant lung volume (e.g., V20 of 10% or larger) must be completed at least 4 weeks prior to first dose of study drug. Radiotherapy that does not include significant lung volume must be completed at least 2 weeks prior to first dose of study drug.

Prior chemotherapy and/or investigational drugs must have been administered at least 4 weeks prior to first dose of study drug.

Sexually active women of childbearing potential (WOCBP) or men who are sexually active with WOCBP must use an effective method of birth control, as defined in section 10.6.3, during the course of the study, in a manner such that risk of failure is minimized. Prior to study enrollment, women of childbearing potential must be advised of the importance of avoiding pregnancy during trial participation and the potential risk factors for an unintentional pregnancy. All women of childbearing potential MUST have a negative pregnancy test within 7 days prior to first dose.

Subject Exclusion Criteria: Subjects who meet any of the following criteria will be excluded from participation in this study:

Patients with active or recent history of known or suspected autoimmune disease that required systemic treatment within the 12 months prior to first dose of study treatment. Subjects with Type 1 diabetes mellitus, residual hypothyroidism due to autoimmune thyroiditis only, requiring hormone replacement, or skin disorders (vitiligo, psoriasis, or alopecia) not requiring systemic treatment, are permitted to enroll.

Patients with a condition requiring systemic treatment with either corticosteroids (>10 mg daily prednisone equivalent) or other immunosuppressive medications within 14 days prior to first dose of study treatment. Inhaled or topical steroids are permitted in the absence of active autoimmune disease.

The presence of an activating EGFR mutation or ALK gene rearrangement, based on patient's medical records.

NSCLC mixed with small cell lung cancer, by pathology.

Prior therapy with anti-programmed death-1 (PD-1), anti-programmed cell death ligand 1 (PD-L1), anti-cytotoxic T lymphocyte-associated antigen 4 (CTLA-4) antibody, or any other antibody or drug specifically targeting T-cell co-stimulation or checkpoint pathways.

All toxicities attributed to prior anti-cancer therapy (other than alopecia, fatigue and Grade 2 peripheral neuropathy) must be resolved to grade 1 (NCT CTCAE version 4) or baseline before administration of study drug.

Patients with a diagnosis of clinically relevant interstitial lung disease.

Known history of testing positive for human immunodeficiency virus (HIV) or known acquired immunodeficiency syndrome (AIDS).

Positive test for hepatitis B virus surface antigen (HBV sAg) or hepatitis C virus ribonucleic acid (HCV RNA), indicating acute or chronic active infection, within 6 months prior to screening NOTE: patients with serology positive for HBV indicating past exposure but without evidence for active infection (e.g. negative PCR) are eligible.

History of severe hypersensitivity reactions to other monoclonal antibodies.

History of other clinically active malignancy within 5 years prior to enrollment, except for tumors with a negligible risk for metastasis or death, such as superficial resected basal-cell carcinoma or squamous-cell carcinoma of the skin or locally ablated/resected carcinoma in situ of the cervix or breast.

Major surgery (including open biopsy) within 4 weeks prior to the start of study, or anticipation of the need for major surgery during study treatment period. Patients must have recovered from the effects of major surgery or significant traumatic injury at least 14 days before the first dose of study treatment.

Minor surgical procedures, within 24 hours prior to the first study treatment.

Breast feeding women.

New York Heart Association (NYHA) Grade II or greater congestive heart failure.

History of myocardial infarction or unstable angina within 6 months prior to first dose of study treatment.

History of stroke or transient ischemic attack within 6 months prior to first dose of study treatment.

History of hemoptysis (≥1/2 teaspoon of bright red blood per episode) within 6 months prior to first dose of study treatment.

Patient with known proliferative and/or vascular retinopathy (e.g. diabetic patients).

Known CNS disease, except for treated brain metastasis: Treated brain metastases are defined as having no evidence of progression or hemorrhage ≤grade 1 (NCT CTCAE version 4) at least four weeks after treatment, as ascertained by clinical examination and brain MM during the screening period. CNS metastases must be asymptomatic and patients have neurologically returned to baseline at least 2 weeks prior to study treatment initiation. In addition, patients must be either off corticosteroids, or on a stable or decreasing dose of <10 mg daily prednisone (or equivalent). Patients with CNS metastases treated by neurosurgical resection or brain biopsy performed within 3 months prior to day of enrollment, will be excluded.

Significant vascular disease (e.g., aortic aneurysm, requiring surgical repair or recent peripheral arterial thrombosis) within 6 months prior to first dose of study treatment. NOTE: Stable peripheral vascular disease is allowed.

Clinical evidence of bleeding diathesis or significant coagulopathy (in the absence of therapeutic anticoagulation).

History of abdominal fistula or gastrointestinal perforation within one year prior to first dose of study drug.

Serious, non-healing wound, active ulcer, or untreated bone fracture.

Patients who received anti-angiogenic therapy within the previous 4 weeks for a tyrosine kinase inhibitor (TKI) or 6 weeks for antibody-based therapy. NOTE: prior bevacizumab treatment is allowed after a 4-week washout period.

Tumor invading major blood vessels (aorta, vena cava, main pulmonary vessels, etc.) or the pericardium or the heart.

Prohibited treatments and/or restricted therapies:
Ongoing or planned administration of anti-cancer therapies other than those specified in this study.
Strong CYP3A4 inhibitors.

Prior treatment with VB-111

Any other serious or uncontrolled medical disorder, active infection, physical exam finding, laboratory finding, altered mental status, or psychiatric condition that, in the opinion of the investigator, would limit the patient's ability to comply with the requirements, substantially increase risk to the patient, or impact the interpretability of study results.

Inability to comply with study and/or follow-up procedures

### Treatment Assignment Procedures

All subjects referred for possible participation in the study must be screened by the investigator to determine the subject's eligibility. Written informed consent must be obtained prior to carrying out any screening procedure. A unique number will be assigned once the subject has signed an informed consent form (ICF). All subjects will be identified by this assigned number and their initials. The unique subject identification number is constructed from a four-digit number, with the first and second digits indicating the study site and the third and fourth digits indicating the subject's number at the site. The first included subject will be subject number 01, the second 02, etc.

Subjects who fail to meet the entrance criteria at any stage during the screening period are defined as screen failures. All screen failures will be documented on the screening log including the reason(s) for screen failure. The screening log will be kept in the Investigator's Site File as per ICH GCP guidelines. The estimated rate of screen failures in phase II part of the study is 10% and the drop-out rate is estimated at 2%. Thus, up to 112 subjects are anticipated to be screened to reach the target number of 100 enrolled subjects, ensuring a minimum of 50 evaluable subjects per arm.

The phase II segment of this open-label trial will include randomization (1:1) to treatment arms, Arm 1 or Arm 2. A centralized randomization procedure will be used.

Randomization will be stratified by the following stratification factors:
PD-L1 expression level: <1% versus ≥1%
Smoking status: former light-smoker or non-smoker versus smoker
   Non-smoker, defined as patients who smoked ≤100 cigarettes in their lifetime
   Former light smoker, defined as patients who smoked between >100 cigarettes AND ≤10 pack-years AND quit ≥1 year prior to enrollment
Gender: male versus female.

### Study Intervention

VB-111 Product Description

Ofranergene Obadenovec VB-111 Formulation: VB-111 is formulated as a sterile vector solution. The solution is supplied frozen (below 65° C.), in single use, 10 ml glass vials. Each vial contains 5 mL of vector at a viral titer of 10¹² VP/ml and vehicle (10% glycerol in Phosphate Buffered Saline). The vector solution should be thawed and maintained at 2-8° C. until dilution and at room temperature until dosing.

Ofranergene Obadenovec VB-111 supply: The study drug is packaged in a small sealed carton box: 6 vials in each box. The study site will be supplied with a sufficient quantity of VB-111 to treat the patients. The study drug will be shipped under appropriate storage conditions to a named addressee (pharmacist, or other designee, according to the regulations of the investigational center). Each delivery must be acknowledged by the addressee. The pharmacist or his designee will dispense the drug at the relevant dosing to the investigator.

A dispensing log will be kept by the pharmacist or designee, in which he/she will record the date(s) and quantity of the Investigational Product dispensed for each patient. The inventory documents will be made available to the study monitor who will verify accountability and verify dose during the course of the study. All used and unused containers will be accounted for during the study and will either be returned to the sponsor for destruction or destroyed on site, if approved by the sponsor. A written confirmation of destruction will be delivered.

Ofranergene Obadenovec VB-111 Storage and Stability: Stability studies of VB-111 are ongoing and to date support a shelf-life of 48 months below 65° C. Shelf-life will be described on the paperwork that accompanies the drug shipment for each batch prepared. VB-111 vials should be stored in closed vials frozen (below 65° C.).

Ofranergene Obadenovec VB-111 Preparation: VB-111 preparation will be as shown in the following table:

TABLE 6
Ofranergene Obadenovec VB-111 Preparation

| | | | | | Take | | | | | Injection | Injection |
|---|---|---|---|---|---|---|---|---|---|---|---|
| | | | Vol. | # vials | this vol. | Syringe | | Syringe | | vol. | vol. |
| | Dose | Conc. | VB111 | of | of | type | Vol. | type | Total | (subject | (subject |
| | VPs | VP/ml | in vial | VB111 | VB111 | VB111 | saline | saline | vol. | ≥50 kg) | <50 kg) |
| Cohort | l × 10¹³ | 10¹² | 5 ml | 2 | 2 × 5 ml | 10 ml | 40 ml | 50* | 50 ml | Entire | 35 ml** |
| 2 of | | | | | | | | | | volume | |
| Phase I | | | | | | | | | | (50 ml) | |
| and | | | | | | | | | | | |
| Arm 1 | | | | | | | | | | | |
| of | | | | | | | | | | | |
| Phase II | | | | | | | | | | | |
| Cohort | 3 × 10¹² | 10¹² | 5 ml | 2 | 2 × 5 ml | 10 ml | 40 ml | 50* | 50 ml | 16.5 ml | 11.5 ml** |
| 1 | | | | | | | | | | | |

*The pharmacist can either use a sterile empty bag and individually add 40 ml Normal Saline (NS) + 10 ml VB-111 to the bag; or use a 50 ml bag of NS and remove the excess volume then add the VB-111.
**35/11.5 ml for patients <50 kg represents a 30% reduction of VB-111.

The entire process of drug preparation shall be carried out at room temperature in the biosafety cabinet (BSC) type II. After thawing, the drug should be diluted in room temperature saline, as soon as possible. Note that if needed, the drug may be maintained on ice for up to 3 hours before the dilution. Once the drug is in its final formulation in saline, keep at room temperature.

VB-111 Dosage and Administration: VB-111 will be intravenously administered at a rate of 3 ml/min, on Day 1 of every fourth treatment cycle (56±5 days). No need for fasting prior to VB-111 dosing. An infusion pump can be used. On days and in cohorts where Nivolumab and VB-111 are both administered, Nivolumab will be administered first.

The maximum time for drug in saline is 60 minutes (plus a 30 minute window) at room temperature. Patients who weigh less than 50kg will receive VB-111 at a reduced dose as shown in Table 6.

On dosing days where the patient is treated with both VB-111 and Nivolumab, Nivolumab shall be prepared and dosed prior to VB-111. This is based on the paradigm that the investigative agent should be given last as a safety precaution. There is no anticipation that there will be sequence-dependent alteration in pharmacology of the two agents. Although this is anticipated to be immediately (within 1 hour) after Nivolumab, it may be administered later (within 24 hours), if clinically indicated, and discussed with the Sponsor's Medical Monitor if longer is required

### Nivolumab Product Description

Nivolumab formulation, dose, and administration: Nivolumab is a fully human monoclonal antibody indicated for the treatment of patients with metastatic NSCLC, with progression on or after platinum-based chemotherapy. The antibody blocks programmed death receptor-1 (PD-1) activity, resulting in decreased tumor growth.

Nivolumab is a sterile, preservative-free, non-pyrogenic, clear to opalescent, colorless to pale yellow liquid that may contain light (few) particles. Nivolumab injection for intravenous infusion is supplied in single-use vials (either 40 mg/4 mL or 100 mg/10 mL solution). Each mL of Nivolumab solution contains Nivolumab 10 mg, mannitol (30 mg), pentetic acid (0.008 mg), polysorbate 80 (0.2 mg), sodium chloride (2.92 mg), sodium citrate dihydrate (5.88 mg), and Water for Injection, USP. May contain hydrochloric acid and/or sodium hydroxide to adjust pH to 6.

A 3 mg/kg dose will be intravenously administered over 60 minutes, on Day 1 of every 14-day cycle. Infusion will be administered through an intravenous line containing a sterile, nonpyrogenic, low protein binding in-line filter (pore size of 0.2 micrometer to 1.2 micrometer). Do not coadminister other drugs through the same intravenous line. Flush the intravenous line at end of infusion.

Nivolumab acquisition: Nivolumab will be prescribed for patients as part of their standard of care treatments.

Nivolumab storage and stability: The product does not contain a preservative. After preparation, store the Nivolumab infusion either
at room temperature for no more than 4 hours from the time of preparation. This includes room temperature storage of the infusion in the IV container and time for administration of the infusion or
under refrigeration at 2° C. to 8° C. (36° F.-46° F.) for no more than 24 hours from the time of infusion preparation.

Do not freeze.

Premedication for VB-111

Anti pyretic Treatment: Acetaminophen (900-1000 mg) will be administered 1-2 hours prior to VB-111 dosing and followed by 450-500 mg acetaminophen, as needed, post-dosing for up to 36 hours.

Corticosteroid Treatment: In patients who develop a grade 3 fever following VB-111 administration, or at investigator's discretion, Dexamethasone 10 mg may be administered 30 minutes (up to 3 hours prior treatment but not sooner than 20 minutes) prior to dosing, in subsequent VB-111doses. Further corticosteroid treatment will be administered at Investigator's discretion.

No pre-medication is indicated for Nivolumab.

Population: A maximum of 112 previously-treated advanced or metastatic non-squamous cell NSCLC patients (≥18 years) who meet the eligibility criteria will be enrolled in the study. Phase I: A minimum of 6 and maximum of 12 patients will be enrolled. Phase II: 100 patients will be enrolled and randomly assigned (1:1) to one of the two treatment arms (50 patients per arm).

Number of sites: Phase I: two sites in Israel; Phase II: additional sites might be opened as deemed necessary.

Study Objectives: Safety: To examine the safety and tolerability of the combination of intravenous administration of VB-111 and Nivolumab, compared to Nivolumab alone, in patients with advanced or metastatic non-squamous cell NSCLC. Efficacy: To evaluate the efficacy of the combination of intravenous administration of VB-111 and Nivolumab, compared to Nivolumab alone, in patients with advanced or metastatic non-squamous cell NSCLC.

### Study Endpoints:

Safety Endpoints: Treatment safety and tolerability will be evaluated based on DLT, AEs, serious adverse events (SAEs), patient clinical status and standard laboratory test results collected before, at regular intervals during the treatment period and for up to 60 days after discontinuation of treatment. Safety evaluations will consist of:
Medical interviews
Monitoring and evaluation of adverse events
Physical examinations
Vital signs
ECG
Laboratory measurements
clinical chemistry, hematology, urinalysis.

The severity/intensity of adverse events will be graded using the Common Terminology Criteria for Adverse Events of the US National Cancer Institute.

Efficacy Endpoints:

Primary Endpoints:
Objective response rate (ORR) by RECIST 1.1

Secondary Endpoints:
Overall survival (OS)
OS rate (at 12 months post-randomization)
ORR as defined by irRECIST
Duration of objective response (DOR)
Time to response (TTR)
Progression-free survival (PFS)
PFS rate (at 12 months post-randomization) Exploratory and sub-study Endpoints:
OS (months) as function of pre-treatment PD-L1 expression
PFS (months) as function of pre-treatment PD-L1 expression
ORR as function of pre-treatment PD-L1 expression
Patient-reported outcomes: Lung cancer symptom scale (LCSS)
Comparison of primary and secondary efficacy endpoints between subgroup of patients who developed at least one post-treatment fever to those who did not.

The following samples will be collected from all patients, for exploratory analyses:
Archival tumor tissue for histopathology
If the Investigator determines that a biopsy is clinically warranted as part of standard of care treatment for a study patient during participation or within 3 months following study drug discontinuation, biopsy samples may be used for further testing for histopathology, for evidence of anti-tumor activity, immune-therapeutic activity and viral transgene.

### Study Procedures/Evaluations

Medical History and Subject Status: Relevant medical and medications history will be obtained by interview or based on medical records at the Screening visit and on Day 1 of each 14-day cycle, starting from Cycle 2. The data collection will confirm histological diagnosis and PD-L1 protein expression (the most recent test prior to study enrollment) and will focus on previous pertinent medical conditions and treatments, concomitant medications and concurrent illnesses. Medical records will be reviewed for documentation of contraindicated diseases. In addition, subjects will be asked to provide a list of current or planned medications (prescription and over-the-counter) and procedures. Archival tumor tissue will be collected from all eligible patients, at the Screening visit.

Physical Examination: A physical examination will be performed within 7 days prior to Day 1 of the first treatment cycle, on Day 1 of each 14-day treatment cycle and 30±7 days from the last dose of the study medication. Weight will be measured at each physical examination and height will only be measured during the screening physical examination.

Vital Signs and Oxygen Saturation: Vital signs and oxygen saturation will be measured within 7 days prior to Day 1 of the first treatment cycle and then on Days 1 and 8 of cycle 1. From cycle 2 onwards those parameters will be measured on Day 1 of each 14-day treatment cycle and 30±7 days from the last dose of the study medication.

Blood pressure, body temperature, respiration and heart rate, 02 saturation by pulse oximetry (and will monitor amount of supplemental oxygen, if applicable) will be recorded 30 minutes (+/−5 min) prior to each dosing, 30 minutes (+/−5 min) after each dosing and at 4 hours (+/−5 min) and 6 hours (+/−60 min) flowing the first VB-111 dose only. Those parameters will be also recorded at any time a patient has any new or worsening respiratory symptoms.

Archival Tumor Tissue: All efforts will be made to obtain and submit tissue from prior surgery for correlative studies. Submission of archived tissue or slides is to occur within 30 days after randomization (please see study lab reference manual). Samples will be subjected to immuno-histochemistry staining, with antibodies against CD4 and CD8 T-cells. VBL retains the option to extend the analysis for additional testing to support further elucidation of the mechanism of action and to identify subsets of patients likely to respond to VB-111

Optional Fresh Biopsy Sample: If the Investigator determines that a biopsy is clinically warranted as part of standard of care treatment for a study patient during participation or within 3 months following study drug discontinuation, those biopsy samples may be used for further tests by VBL (for evidence of anti-tumor activity and immune-therapeutic activity and viral transgene). If a sample of tissue is collected, the residual tissue will be prepared into 3 samples:
One part of the tissue (˜60 mL) will be placed immediately in 30 mL of 10% formalin and shipped ambient within one week of collection to VBL for processing, preferably after block preparation. Samples will be stored under ambient conditions at VBL or at a central lab assigned by VBL.
Two parts of the tissue will be prepared as fresh pieces, immediately snap-frozen in liquid nitrogen in two 2-mL cryovials and shipped frozen to VBL. Frozen samples will be shipped to VBL, once every three months, with dry ice, and will be stored in the central lab in a nonfrost freezer at −70 ° C. or below.

Among other tests (anti-tumor activity and immune-therapeutic activity) VBL will also explore and validate the presence and expression of viral transgene in the tumor tissue. DNA and/or RNA will be extracted from the fresh frozen tissue sample using DNA and/or RNA isolation kits. DNA samples will be tested by PCR for the presence of the sequence of the inserted viral trans-gene in the tissue. RNA samples will be tested by PCR for viral trans-gene expression in the tissue.

ECOG Performance Status: Will be evaluated within 7 days of Day 1 of the first treatment cycle, on Day 1 of each 14-day treatment cycle and 30±7 days from the last dose of the study medication.

Electrocardiogram: A 12-lead ECG will be performed within 7 days of Day 1 of Cycle 1 and 30±7 days after last dose. The Investigator will report whether the ECG is normal or abnormal and its clinical significance. All clinically significant abnormalities found at screening should be documented in the CRF as medical history.

Clinical Laboratory Evaluations: Laboratory tests for eligibility, safety and impact of treatment will be performed as per local standard of care and clinical indication, at a local laboratory and results will be recorded in the study database. Local laboratory reference ranges for all test parameters and relevant laboratory certificates should be provided to the CRO prior to receiving study medication shipment.

Hematology: Hematological assessments will be performed within 7 days of initiation of cycle 1, on Days 1 and 8 of Cycle 1, on Day 1 of every subsequent 14-day cycle and 30±7 days after last dose (always before dosing). A complete blood count (CBC) will include assessment of hemoglobin, hematocrit, white blood cells (WBCs) with complete manual or automated differential (total neutrophils, lymphocytes, monocytes, eosinophils, basophils; absolute or percentage will be acceptable), red blood cells (RBCs), platelet count and erythrocyte sedimentation rate (ESR)

Biochemistries: Biochemistry assessments will be performed within 7 days of initiation of cycle 1, on Days 1 and 8 of Cycle 1, on Day 1 of every subsequent 14-day cycle and 30±7 days after last dose (always before dosing). Assessments will include evaluation of a comprehensive metabolic panel (alanine aminotransferase (ALT), aspartate aminotransferase (AST), alkaline phosphatase (ALP), albumin, calcium, magnesium, sodium, potassium, gamma-glutamyl transferase (GGT), total bilirubin, creatinine, creatinine clearance, differential total protein, uric acid, urea (BUN), glucose and international normalized ratio (INR). Liver function test results must be obtained within 72 hours before dosing (can be done on the day of dosing but not more than 3 days before dosing).

Thyroid function: Thyroid function will be assessed on Day 1 of cycle 1, on Day 1 of every second treatment cycle (i.e. every 4 weeks) and 30±7days following last dose. TSH will be assessed and if any abnormalities are recorded, reflex to T3 and free T4 will be measured as well.

Urinalysis: General urinalysis will be assessed within 7 days of first treatment dose, on Day 1 of each treatment cycle, starting from treatment cycle 2, and 30±7days following last dose. Tests will include proteinuria testing, to be performed according to local standards. In case of new or increased proteinuria, 24 h urine collection may be required. A +2 dipstick result will require a 24-hour collection but +3 dipstick result will require holding study drug and a 24-hour collection. Pyuria in the presence of elevated creatinine will require evaluation of possible nephritis.

Coagulation function: Prothrombin time (PT) and partial thromboplastin time (PTT) (in seconds) will be assessed within 7 days of first treatment dose, on Day 1 of each treatment cycle, starting from treatment cycle 2, and 30±7days following last dose. Prolongation beyond 20% of the ULN, will require withholding of VB-111 dosing, unless the Investigator believes the increase is due to anticoagulant use.

Pregnancy test: A serum or urine hCG pregnancy test will be performed in women with child-bearing potential within 7 days prior to initiation of first treatment dose. Subsequent testing will be performed on Day 1 of every second treatment cycle (every 28 days) prior to dosing. A negative result must be available prior to administration VB-111 treatment. Pregnancy tests are not required for women unable to become pregnant for one of the following reasons:
Menopause confirmed by healthcare provider
The woman has had her uterus or both ovaries or both fallopian tubes removed

Computerized tomography (CT): A CT scan of the chest, abdomen and pelvis and any area that is being monitored at screening and during treatment and of additional sites of known or suspected disease (including CNS) will be collected within 28 days of first study dose and on Day 1 of every fourth treatment cycle (every 8 weeks±5 days). After discontinuation of study medication, every effort will be made to collect post-treatment scans until death, withdrawal of consent or lost to follow up. For patients who discontinue study treatment for reason other than PD, follow up scans will be performed every 8 weeks (±7 days), as per standard of care, until PD, withdrawal of consent, death, lost to follow-up. Tumor assessment at screening will be by institutional standards CT. Method of tumor assessment should be consistent throughout all visits and performed until disease progression. Patients will be assessed for disease response or progression, by the investigator, throughout the study according to RECIST 1.1 and according to irRECIST. CT scans will be collected for central lab review but only for patients from Phase II part of the study and only on a “collect and store” basis. CDs will need to be collected per each CT and stored at the patient's file for later analysis by the Sponsor. In the meanwhile and during the ongoing course of the study, the CTs will be read and analyzed by the Investigator. The responsibility to determine response based on scans in real time during the study will lay on the rather than on the central reader.

Patient-reported outcomes: Patients will complete the Lung Cancer Symptom Scale (LCSS) on Day 1 of Cycle 1, on Day 1 of every fourth cycle (every 8 weeks±5 days) and on Day 30±7 post last dose. Thereafter, every effort will be made to complete post-treatment LCSS every 8 weeks±7 days, until death, withdrawal of consent or lost to follow up. In general, patients will be asked to complete the LCSS prior to any study procedures and study treatment.

### Study Schedule

Screening (Day −1 to −28)

The Screening period for a particular subject commences once the subject signs the informed consent form (ICF). Written informed consent must be obtained before any protocol-specific tests or procedures may be conducted. After informed consent is obtained, the Screening assessments will be performed within 28 days of the planned initiation of treatment, except for tests to be performed within 7 days of starting treatment as indicated below. Standard of care tests, including physical examination and blood tests, that were conducted prior to ICF may be used for Screening.

A unique subject number will be assigned at the time of Screening that will be used to identify the subject throughout the clinical study and must be used on all study documentation related to that subject.

The following assessments will be performed at the Screening visit:
Obtain and document consent from potential subject on ICF
Obtain a medical history by interview and/or medical chart review, to determine eligibility
Review medications history by interview and/or medical chart to determine eligibility
Obtain archival tumor tissue from patients who meet the eligibility criteria.
Assess tumor status per RECIST 1.1 guidelines

The following tests must be performed no more than 7 days prior to first dosing session:
Perform a full body physical examination, including weight, height, vital signs and oxygen saturation
Perform a 12-Lead ECG
Assess ECOG performance status
Perform hematological, biochemical, coagulation, thyroid function and urinalysis tests.
Perform a urine pregnancy test for women of child-bearing potential when applicable.

### Study Visits

Cycle 1, Day 1—Baseline
Review and record concomitant medications to confirm eligibility
Measure vital signs and oxygen saturation
Perform hematological, biochemical, and thyroid function tests
Have patient complete LCSS questionnaire
Perform tumor biopsy, if indicated.
Confirm eligibility based on inclusion/exclusion criteria
Administer first dose of study treatment. Patients receiving both Nivolumab and VB-111, Nivolumab is infused first.
Record adverse events

Cycle 1, Day 8 (±1 day)
Review and record concomitant medications
Record adverse events
Measure vital signs and oxygen saturation
Perform hematological and biochemical, coagulation, thyroid function and urinalysis tests
Perform tumor biopsy, if indicated. Cycles 2 and above, Day 1 (±5 days)
Assess adverse events as reported by subject or observed by investigator
Review and record concomitant medications
Perform a full body physical examination, including weight, vital signs and oxygen saturation
Assess ECOG performance status
Perform hematological, biochemical, thyroid function, coagulation, urinalysis tests.
Perform serum or urine pregnancy test (every second cycle), in women of child-bearing potential.
Perform tumor biopsy, if indicated.
Administer study treatment.

To be performed every fourth cycle (Cycles 4, 8, 12, etc.) only:
Have patient complete LCSS questionnaire before performing any other tests or procedures
Collect CT scan and assess tumor response, per RECIST 1.1 and irRECIST guidelines
Record adverse events reported by subject or observed by investigator
Record concomitant medications Study completion visit −30 days (±7 days) following last dose
Record adverse events reported by subject or observed by investigator. AEs recording will continue until 60 days post last dose.
Record concomitant medications
Have patient complete LCSS questionnaire before performing any other tests or procedures
Perform a physical examination, including weight, vital signs and oxygen saturation
Assess ECOG performance status
Perform an ECG
Collect blood samples for hematological, biochemical, thyroid function, coagulation tests.
Collect urine for urinalysis test

Post-study surveillance
Every 8 weeks (±7 days) following discontinuation

Every effort will be made to continue follow-up of all treated patients, until death, withdrawal of consent or loss to follow-up.
Record adverse events as reported by subject or observed by investigator (up to 60 days after last dose only)
Record any further anti-cancer therapies
Have patient complete LCSS questionnaire
Collect CT scan and assess tumor response, per RECIST 1.1 and irRECIST guidelines
Vital status, can be collected by phone. s

Efficacy Evaluation Criteria

Parameters of Response—RECIST 1.1:

Measurable disease is defined as at least one lesion that can be accurately measured in at least one dimension (longest diameter to be recorded). Longest diameter of each lesion must be □10 mm, measured by CT scan using contrast. CT scan slice thickness should be no greater than 5 mm. Chest, abdomen and pelvic CTs should be performed at each time-point. The same method should be used for tumor assessment throughout the study.

Malignant lymph nodes should be considered as measurable disease if their short axis is >15 mm.

All measurable lesions up to a maximum of 2 lesions per organ and 5 lesions in total representative of all involved organs should be identified as target lesions and will be recorded and measured at baseline.

Target lesions should be selected on the basis of their size (lesions with the longest diameter), being representatives of all involved organs, and their suitability for accurate reproducible repetitive measurements by one consistent method of assessment (either by imaging techniques or clinically). A sum of the longest diameter (LD) for all target lesions will be calculated and reported as the baseline sum LD.

All other lesions (or sites of disease) should be identified as non-target lesions and should also be recorded at baseline. Measurements are not required and these lesions should be followed as “present”, “absent” or “unequivocal progression”.

All baseline evaluations of disease status should be performed as close as possible to the start of treatment and never more than 4 weeks before the beginning of treatment.

Response Criteria: Measurement of the longest diameter of each target lesion is required for follow-up. For lymph-nodes, measurement of the smallest diameter is required for follow-up. Change in the sum of these diameters affords some estimate of change in tumor size and hence therapeutic efficacy. All lesions must be assessed using the same technique as baseline.

Complete Response (CR): is disappearance of all target and non-target lesions and no evidence of new lesions. Any pathological lymph nodes must have reduction in short axis to <10 mm. CR must be documented by two disease assessments, at least 4 weeks apart.

Partial Response (PR): is at least a 30% decrease in the sum of longest diameters (LD) of all target measurable lesions taking as reference the baseline sum of LD. There can be no unequivocal progression of non-target lesions and no new lesions. Documentation by two disease assessments at least 4 weeks apart is required. In the case where the ONLY target lesion is a solitary pelvic mass measured by physical exam, which is not radiographically measurable, a 50% decrease in the LD is required.

Progressive Disease (PD): is at least a 20% increase in the sum of LD of target lesions taking as references the smallest sum LD. The sum must demonstrate an absolute increase of at least 5 mm. The appearance of one or more new lesion is also considered increasing disease. Unequivocal progression of existing non-target lesions, other than pleural effusions without cytological proof of neoplastic origin, in the opinion of the treating physician within 12 weeks of study entry is also considered increasing disease (in this circumstance an explanation must be provided). In the case where the ONLY target lesion is a solitary pelvic mass measured by physical exam, which is not radiographically measurable, a 50% increase in the LD is required.

Stable Disease: is any condition not meeting the above criteria.

Inevaluable for response: is defined as having no repeat tumor assessments following initiation of study therapy for reasons unrelated to symptoms or signs of disease.

Parameters of Response
irRECIST Criteria:

In addition to evaluation using RECIST 1.1 criteria, an immune response adaptation of RECIST will be applied to this trial. Both RECIST 1.1 and irRECIST should be evaluated in this study for each CT. The irRECIST will be used for decisions regarding treatment cont./discot. The essential differences between irRECIST and RECIST criteria are as follows:
New measureable lesions do not necessarily constitute progressive disease and they should be added into the total tumor burden. New non-measurable lesions do not constitute disease progression but will prevent the determination of an irCR.
Apparent disease progression should be confirmed after 4 weeks in the absence of symptoms consistent with clinical deterioration.

At baseline, the sum of the longest diameters (SumD) of all target lesions (up to 2 lesions per organ, up to total 5 lesions) is measured. At each subsequent tumor assessment (TA), the SumD of the target lesions and of new, measurable lesions (longest diameter >10 mm [lymph nodes ≥15 mm in shortest diameter]; up to 2 new lesions per organ, total 5 new lesions) are added together to provide the total measurable tumor burden (TMTB): TMTB=SumD target lesions+SumD new, measurable lesions.

Percentage changes in TMTB per assessment time point describe the size and growth kinetics of both old and new, measurable lesions as they appear. At each tumor assessment, the response in target and new, measurable lesions is defined based on the change in TMTB (after ruling out irPD) as follows:

Complete Response (irCR): complete disappearance of all target and new, measurable lesions, with the exceptions of lymph nodes which must decrease to <10 mm in short axis

Partial Response (irPR): decrease in TMTB ≥30% relative to baseline (see below)

Stable Disease (irSD): not meeting criteria for irCR or irPR, in absence of irPD

Progressive Disease (irPD): increase in TMTB ≥20% relative to nadir. Unless there is a rapid clinical deterioration, irPD should be confirmed with a second, consecutive scan obtained ≥4 weeks from the initial irPD documentation. Once confirmed, irPD date will be considered as the date of initial irPD documentation.

Overall response according to irRECIST is derived from the responses in measurable lesions (based on TMTB) and the presence of any non-measurable lesions.

Additional Parameters of Response

Overall Survival is the observed length of life from first administration of VB-111 to death or the date of last contact.

Progression-Free Survival (measurable disease studies) is the period from first administration of VB-111 until disease progression, death or date of last contact.

Overall Response Rate (ORR) is the proportion of complete response [CR] and partial response [PR]

Duration of Response (DOR) is the time from first evidence of PR or better to confirmation of PD or death due to any cause. DOR will be calculated for subjects who achieve CR or PR.

Time to Response (TTR) is the time from initiation of treatment to documented PR or better.

### Statistical Methods

All data collected will be summarized and presented. Continuous variables will be described as the mean, median, standard deviation, and range of n observations Categorical data will be described with contingency tables including frequency and percentage. Individual patient listings of all data will be generated and presented. Statistical tests comparing the treatment groups will be performed at the two-sided 5% level. Statistical descriptions and analyses will be carried out using R version 3.4.3 (R Development Core Team. Vienna, Austria).

Study Populations: The safety population will include all subjects who received at least one dose of study medication. All safety analyses will be performed on the safety population. The Modified Intent to Treat (mITT) population will include all subjects from the safety population who had at least one post baseline efficacy measurement (RECIST 1.1). Efficacy analysis will be performed on the mITT population In addition, a separate analysis will include the mITT subjects and the subjects from part I of the study.

Demographic and Baseline parameters: Demographic and baseline parameters will be summarized overall and by treatment group. All continuous variables will be summarized by descriptive statistics. All discrete variables will be summarized by frequencies and percentages.

Study Duration and Compliance: All study drug administration and compliance data will be summarized.

Prior and Concomitant Medication: All relevant prior medication and all concomitant medications will be summarized by frequencies and percentages. All medications will be coded using the World Health Organization (WHO) drug dictionary.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

### Example 4

A Phase II trial of VB-111 in Combination with Nivolumab in Patients with Metastatic Colorectal Cancer (mCRC).

### Background:

Immune based approaches in GI cancers have unfortunately—with the notable exception of immune checkpoint inhibition in microsatellite instable (MSI-H) disease and gastric cancer—been largely unsuccessful. The reasons for this are unclear but no doubt relate to the fact that in advanced disease GI cancer appears to be less immunogenic, as evidenced by the lack of infiltrating lymphocytes with advancing T stage as well as an immunosuppressive tumor micro environment.
VB-111 is an anti-angiogenic agent comprising of a nonreplicating E1 deleted adenovirus type 5 which contains a modified murine preproendothelin (PPE) promoter and Fas-chimera transgene
VB-111 has been tested and shows promise in glioblastoma, ovarian and thyroid tumors
Nivolumab is a human monoclonal antibody directed against PD-1.
The aim of this study is to study the effects of VB-111 in colorectal cancer (CRC) and to evaluate whether the antitumor immunity induced by VB-111 therapy can be enhanced by PD-1 inhibition.

### Objectives:

To determine the safety and tolerability of VB-111 in combination with nivolumab in patients with refractory, metastatic CRC.
To determine Best Overall Response (BOR) (partial response (PR) +complete response (CR)) according to Response Evaluation Criteria (RECIST v1.1) of combined treatment of VB-111 and nivolumab in patients with refractory, metastatic CRC.

### Eligibility:

Histopathological confirmation of colorectal cancer metastatic to the liver.
Patients must have progressed on >2 lines of standard of care chemotherapy for colorectal cancer or been intolerant of chemotherapy or refused prior chemotherapy.
Patients tumors must be documented to be microsatellite stable (MSS).
Patients must have at least 1 focus of metastatic disease that is amenable to pre- and on-treatment biopsies and be willing to undergo this.
All patients enrolled will be required to have measurable disease by RECIST v 1.1 criteria.

### Design:

The proposed study is a phase II study of VB-111 in combination with immune checkpoint inhibition (nivolumab) in patients with metastatic CRC
Treatment will be delivered in cycles consisting of 2 weeks with VB-111 given every 6 weeks and nivolumab given every 2-week until progression or unacceptable toxicity.
Disease status evaluation will be done every 8 (+/−1) weeks after the start of study therapy.

### Inclusion Criteria

Patients must have histopathological confirmation of colorectal cancer by the Laboratory of Pathology of the NCI.
Patients must have radiologically confirmed liver metastasis.
Patients must:
   have progressed on >2 lines of standard of care chemotherapy for colorectal cancer OR
   been intolerant of standard of care chemotherapy for colorectal cancer OR
   refused prior standard of care chemotherapy for colorectal cancer.
Patients who have a known KRAS wild type tumor must have progressed, been intolerant of or refused anti-EGFR based treatment.
Patients tumors must be documented to be microsatellite stable (MSS).
Patients must have at least 1 focus of metastatic disease that is amenable to pre- and on-treatment biopsies and be willing to undergo this. Ideally, the biopsied lesion should not be one of the target measurable lesions, although this can be up to the discretion of the investigators
Patients must have measurable disease by RECIST v 1.1 criteria.
Age≥18 years. Because no dosing or adverse event data are currently available on the use of nivolumab in combination with VB-111 in patients <18 years of age, children are excluded from this study, but will be eligible for future pediatric trials.
ECOG performance status.
Adequate hematological function defined by:
   white blood cell (WBC) count≥3×10⁹/L
   absolute neutrophil count (ANC)≥1.5 ×10⁹/L
   lymphocyte count≥0.5×10⁹/L
   platelet count≥100×10⁹/L
   Hgb≥9 g/dL (more than 48 hours post-completion of blood transfusion))
PT and PTT (seconds) <1.2×ULN. Patients who are anticoagulated do not need to meet criteria for PT and PTT
INR, fibrinogen <1.2×ULN. Patients who are anticoagulated do not need to meet criteria for INR.
Adequate hepatic function defined by:
   a total bilirubin level <1.5×ULN,
   an AST level ≤2.5×ULN in the absence of hepatic metastasis; or ≤5×ULN in the presence of hepatic metastases,
   an ALT level ≤2.5×ULN in the absence of hepatic metastasis; or ≤5×ULN in the presence of hepatic metastases
Adequate renal function defined by:

| | |
|---|---|
| Creatinine OR | <1.5x institution upper limit of normal OR |
| Measured or calculated | ≥50 mL/min/1.73 m² for participant with |
| creatinine clearance (CrC1) | creatinine levels |
| (eGFR may also be used in | ≥1.5 X institutional ULN |
| place of CrC1) ^{A} | |

^{A} Creatinine clearance (CrC1) or eGFR should be calculated per institutional standard.

The effects of nivolumab and VB-111 on the developing human fetus are unknown. For this reason, women of child-bearing potential and men must agree to use adequate contraception prior to study entry and for the duration of study participation and up to 5 months (women) and 7 months (men) after the last dose of the nivolumab or 2 months after the last dose of VB-111 whichever is the longer time period. Should a woman become pregnant or suspect she is pregnant while she or her partner is participating in this study, she should inform her treating physician immediately.
Troponin level in normal range at the time of enrollment.
Patient must be able to understand and willing to sign a written informed consent document.
Weight>35kg
Patients must be enrolled in tissue collection protocol.

### Exclusion Criteria

Patients who have had standard-of-care anti-cancer therapy or therapy with investigational agents (e.g. chemotherapy, immunotherapy, endocrine therapy, targeted therapy, biologic therapy, tumor embolization, monoclonal antibodies or other investigation agents), large field radiotherapy, or major surgery within 4 weeks prior to enrollment.
Patients who have had anti-VEGF therapy within 4 weeks prior to enrollment.
Patients currently on a corticosteroid dose greater than physiologic replacement dosing defined as 10 mg of cortisone per day or its equivalent.
Patients with known brain metastases because of their poor prognosis and because they often develop progressive neurologic dysfunction that would confound the evaluation of neurologic and other adverse events.
Patients with signs of liver failure, e.g. clinically significant ascites, encephalopathy, or variceal bleeding within 6 months prior to enrollment.
Prior major liver resection: remnant liver <50% of the initial liver volume. Patients with a biliary stent can be included.
Patients with active autoimmune disease or history of autoimmune disease that might recur, which may affect vital organ function or require immune suppressive treatment including systemic corticosteroids. These include but are not limited to patients with a history of immune related neurologic disease, multiple sclerosis, autoimmune (demyelinating) neuropathy, Guillain-Barre syndrome or CIDP, myasthenia gravis; systemic autoimmune disease such as SLE, connective tissue diseases, scleroderma, inflammatory bowel disease (IBD), Crohn's, ulcerative colitis, hepatitis; and patients with a history of toxic epidermal necrolysis (TEN), Stevens-Johnson syndrome, or phospholipid syndrome. Such diseases should be excluded because of the risk of recurrence or exacerbation of disease.
   Of note, patients with vitiligo, endocrine deficiencies including thyroiditis managed with replacement hormones including physiologic corticosteroids are eligible. Patients with rheumatoid arthritis and other arthropathies, Sjogren's syndrome and psoriasis controlled with topical medication and patients with positive serology, such as antinuclear antibodies (ANA), anti-thyroid antibodies should be evaluated for the presence of target organ involvement and potential need for systemic treatment but should otherwise be eligible.
History of idiopathic pulmonary fibrosis (including bronchiolitis obliterans with organizing pneumonia) or evidence of active pneumonitis on screening chest CT scan.
Uncontrolled intercurrent illness including, but not limited to, ongoing or active infection, symptomatic congestive heart failure, unstable angina pectoris, cardiac arrhythmia, or psychiatric illness/social situations (within timeframes identified in the bullets below) that would limit compliance with study requirements.
History of severe or unstable cerebrovascular disease.
Pulse oximetry <92% on room air.
Myocardial infarction within 6 months prior to enrollment.
History of myocarditis.
Sustained hypotension (<90/50 mmHg) or uncontrolled hypertension (>160/100 mmHg)
Stroke within 6 months prior to enrollment.
Patients with proliferative and/or vascular retinopathy.
Significant vascular disorders (e.g. aortic aneurysm, requiring surgical repair or recent peripheral arterial thrombosis) within 6 months prior to enrollment.
History of hemoptysis (>½ teaspoon of bright red blood per episode) or active GI bleeding within 6 months prior to enrollment.
Evidence of a bleeding diathesis or significant coagulopathy (in the absence of therapeutic anticoagulation)
History of abdominal fistula or gastrointestinal perforation
HIV-positive patients are excluded because HIV causes complicated immune deficiency and study treatment can possess more risks for these patients.
Prior autologous or allogenic hematopoietic stem cell transplant.
Subjects with ascites.
Patients with unhealed surgical wounds for more than 30 days.
History of allergic reactions attributed to compounds of similar chemical or biologic composition to nivolumab or VB-111.
History of severe hypersensitivity reaction to any monoclonal antibody.
Prior invasive malignancy (except non-melanomatous skin cancer) unless disease free for a minimum of 3 years prior to enrollment.
Pregnant women are excluded from this study because nivolumab and VB-111 potential for teratogenic or abortifacient effects are unknown. Because there is an unknown but potential risk for adverse events in nursing infants secondary to treatment of the mother with nivolumab and VB-111, breastfeeding should be discontinued if the mother is treated with nivolumab and/or and VB-111.

### Study Design and Treatment Plan

This study will be an open label, single-arm phase II study of VB-111 in combination with anti-PD1 antibody, nivolumab, in patients with advanced, refractory CRC.

Treatment will be delivered in cycles consisting of 2 weeks (+/−3 days).

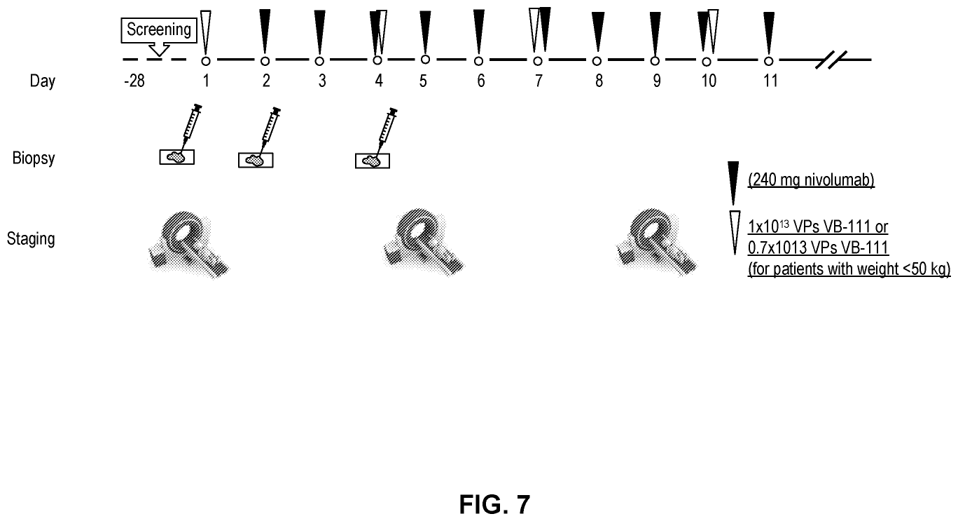

VB-111 will be administered every 6 weeks starting on cycle 1 day 1 and nivolumab will be administered every 2 weeks starting on cycle 2 day 1 (FIG. 7 and Table 7).

Treatment will continue until off treatment criteria are met.

Patients will be monitored every 8 (+/−1) weeks with imaging.

TABLE 7
Treatment regimen

| | Nivolumab, |
|---|---|
| VB-111, every 6 weeks | every 2 weeks |
| 1x10¹³ VP IV (for patients with weight ≥50 kg) | 240 mg IV |
| 0.7x10¹³ VP IV (for patients with weight >35 kg and | |
| <50 kg) | |

### VB-111 Administration

VB-111 will be given on Day 1 of cycle 1 and continue every 3 cycles (cycles 4, 7, 10 and so on) at a flat dose of 1×10¹³ or 0.7×10¹³ VP. VB-11 will be administered over approximately 60-90 minutes via intravenous infusion.

The maximum time for VB-111 from its dilution in 0.9% sodium chloride solution and start of the infusion should be less than 60 minutes at room temperature. Ace

taminophen 500-1000 mg will be administered orally 1-2 hours prior to VB-111 infusion and followed by 325-500 mg as needed every 4-6 hours post treatment up to 36 hours. In patients who develop >grade 3 fever following VB-111 administration or at the discretion of the investigator, dexamethasone IV 10 mg may be administered 20 minutes to 3 hours prior to treatment (but no sooner than 20 minutes) in subsequent VB-111 doses.

### Nivolumab Administration

Nivolumab will be given on day 1 of every cycle starting at cycle 2 at a flat dose of 240 mg. Nivolumab will be administered over approximately 30-60 minutes via intravenous infusion.

Nivolumab will be administered through a 0.2 micron to 1.2-micron pore size, low-protein binding in-line filter.

On days when both drugs are given, VB-111 will be given first. Nivolumab infusion will start approximately 1 hour after the end of VB-111 infusion.

Vital signs will be collected within 1 hour before VB-111 and nivolumab infusions, at least once during each infusion, and within 30 minutes after the completion of the infusion.

For nivolumab, in the event of a <Grade 2 infusion-related reaction, the infusion rate of study drug may be decreased by 50% or interrupted until resolution of the event and re-initiated at 50% of the initial rate until completion of the infusion. Acetaminophen and/or an antihistamine (e.g. diphenhydramine) or equivalent medications per institutional standard may be administered at the discretion of the investigator. If the infusion related reaction is >Grade 3 or higher in severity, study drug will be discontinued.

TABLE 8
Study Calendar

| | | | Cycle | Sub- | 28 | |
|---|---|---|---|---|---|---|
| | | | 1 | sequent | Days | Long |
| | Screen- | Base- | Day | Cycles | Safety | Term |
| | ing¹ | line¹ | 1 ¹ | Day 1 | FU^{10,12} | FU^{11,12} |
| Nivolumab² | | | | X | | |
| VB-111³ | | | Every 6 weeks | | | |
| Microsatellite | X | | | | | |
| stable | | | | | | |
| (MSS) status⁴ | | | | | | |
| Histologic | X | | | | | |
| confirmation | | | | | | |
| of disease | | | | | | |
| Medical History | X | | | | | |
| Height | X | | | | | |
| Physical exam, | X | | X | X | X | X |
| weight | | | | | | |
| and ECOG | | | | | | |
| EKG | X | | X | X | | |
| HIV serology | X | | | | | |
| Cardiology | X | | X | X | | |
| consult ⁵ | | | | | | |
| Ophthalmologic | X | | | | | |
| exam ⁵ | | | | | | |
| Echo- | X | X | | | | |
| cardiogram ⁵ | | | | | | |
| 24-hour urine | X | | | | | |
| (if creatinine | | | | | | |
| clearance is | | | | | | |
| tested this way) | | | | | | |
| TB testing ⁵ | X | | | | | |
| Troponin I | X | | X | X | | |
| PT, INR, PTT, | X | | X | X | X | |
| fibrinogen | | | | | | |
| Urinalysis | X | | | | | |
| ACTH and | X | | | | | |
| morning | | | | | | |
| cortisol | | | | | | |
| Baseline | | X | | | | |
| signs and | | | | | | |
| symptoms | | | | | | |
| HLA | | X | | | | |
| Concomitant | | X | X | X | | |
| medications | | | | | | |
| Vital Signs | X | | X⁶ | X⁶ | X | |
| CBC | X | | X | X | X | X |
| w/differential, | | | | | | |
| Platelets | | | | | | |
| Biochemical | X | | X | X | X | X |
| profile⁷ | | | | | | |
| Thyroid tests | X | | X | X | X | |
| TSH, T3, T4 | | | | | | |
| Uric acid, | X | | X | X | X | |
| amylase | | | | | | |
| and lipase | | | | | | |
| Tumor | X | | X | X | X | |
| marker CEA, | | | | | | |
| AFP, CA19-9 | | | | | | |
| Serum or urine | X | | X | X | | |
| pregnancy test | | | | | | |
| Radiologic | X | X | Every 8 weeks | | | X |
| Evaluation⁸ | | | | | | |
| Adverse event | | | X | X | X | X |
| evaluation | | | | | | |
| Tumor biopsy⁹ | | X | | X | | |
| Phone call or | | | | | | X |
| e-mail for | | | | | | |
| survival every 6 | | | | | | |
| months | | | | | | |

¹Baseline and C1D1 evaluations do not need to be repeated if performed at screening or baseline in designated time frame. All evaluations will be done within 72 hours before treatment initiation on Day 1 of every cycle. If treatment does not start within 28 days after enrollment, screening evaluations will be repeated. Cycle is 14 (+/−3) days.
²240 mg of nivolumab via IV infusion on Day 1 of each cycle starting on cycle 2.
³1 × 10¹³ VP of VB-111 IV on Day 1 of cycle 1 and every +3 cycles (4, 7, 10 and so on). Decreased dose of 0.7 × 10¹³ VP for patients with weight ≥35 kg and <50 kg.
⁴confirmed by genetic analysis or immunohistochemistry.
⁵ if clinically indicated.
⁶Vital signs will be collected within 1 hour before VB-111 and nivolumab infusions, at least once during each infusion, and within 30 minutes after the completion of the infusion.
⁷Biochemical Profile: electrolytes, BUN, creatinine, AST, ALT, total and direct bilirubin, calcium, phosphorus, albumin, magnesium.
⁸CT scan or Mill of chest, abdomen and pelvis on screening, baseline and every 8 (+/−1) weeks after start of study therapy. If treatment continues after initial estimation of PD, conformational scan will be done 4 weeks (+/−1 week) later. If patient is taken off treatment for reason other than disease progression, imaging will continue during Follow UP until disease progression.
⁹Mandatory tumor biopsies will be performed at baseline and on Day 1 of cycle 2 or cycle 4. If the patient's disease progresses before scheduled biopsy, post-treatment biopsy may be performed per PI discretion at the time of progression
¹⁰+/−1 week
¹¹Follow up visits are planned to be performed at 60 (+/−14 days) and 90 (+/−14 days) days after treatment discontinuation to evaluate patient's safety. After this visit, subjects will be followed every 6 months (±1 month) for survival by phone call or e-mail.
NOTE:
if patient is taken off treatment for reason other than disease progression, we will continue to invite patient every 8 (+/−1) weeks for imaging studies. Outside scans are acceptable.
¹²If subjects are not willing to come to NIH for FU visits, they will be contacted by phone call or e-mail for survival and adverse events.

### Response Criteria

For the purposes of this study, patients should be re-evaluated for response every 8 weeks (+/−1 week). Response and progression will be evaluated in this study using the new international criteria proposed by the revised Response Evaluation Criteria in Solid Tumors (RECIST) guideline (version 1.1) and modified immune-related response.

Whilst immune-related RECIST criteria will be taken into consideration regarding continuation of therapy in the event of growth, standard RECIST criteria will be the primary method used for evaluation of the primary endpoint.

The study treatment can continue according to the investigator's decision in case of progressive disease according to RECIST 1.1. For this situation, modified Immune-Related response criteria (irRC) based on RECIST 1.1 in all subjects without worsening of existing symptoms or developing new tumor-related symptoms at the time of progression will be used. s

Response Criteria—Evaluation of Target Lesions

Complete Response (CR): Disappearance of all target lesions. Any pathological lymph nodes (whether target or non-target) must have reduction in short axis to <10 mm.

Partial Response (PR): At least a 30% decrease in the sum of the diameters of target lesions, taking as reference the baseline sum of diameters.

Progressive Disease (PD): At least a 20% increase in the sum of the diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progressions).

Stable Disease (SD): Neither sufficient shrinkage to qualify for PR nor sufficient increase to qualify for PD, taking as reference the smallest sum of diameters while on study.

### Evaluation of Non-Target Lesions

Complete Response (CR): Disappearance of all non-target lesions and normalization of tumor marker level. All lymph nodes must be non-pathological in size (<10 mm short axis). Note: If tumor markers are initially above the upper normal limit, they must normalize for a patient to be considered in complete clinical response.

Non-CR/Non-PD: Persistence of one or more non-target lesion(s) and/or maintenance of tumor marker level above the normal limits.

Progressive Disease (PD): Appearance of one or more new lesions and/or unequivocal progression of existing non-target lesions. Unequivocal progression should not normally trump target lesion status. It must be representative of overall disease status change, not a single lesion increase. Although a clear progression of “non-target” lesions only is exceptional, the opinion of the treating physician should prevail in such circumstances, and the progression status should be confirmed at a later time by the review panel (or Principal Investigator).

### Evaluation of Best Overall Response

The best overall response is the best response recorded from the start of the treatment until disease progression/recurrence (taking as reference for progressive disease the smallest measurements recorded since the treatment started). The patient's best response assignment will depend on the achievement of both measurement and confirmation criteria.

For patients with measurable disease (i.e., target disease), see Table 9.:

TABLE 9

| | | | | Best Overall |
|---|---|---|---|---|
| | | | | Response when |
| Target | Non-Target | New | Overall | Confirmation is |
| Lesions | Lesions | Lesions | Response | Required* |
| CR | CR | No | CR | ≥4 wks. |
| | | | | Confirmation** |
| CR | Non-CR/Non-PD | No | PR | ≥4 wks. |
| CR | Not evaluated | No | PR | Confirmation** |
| PR | Non-CR/Non- | No | PR | |
| | PD/not evaluated | | | |
| SD | Non-CR/Non- | No | SD | Documented at least |
| | PD/not evaluated | | | once ≥4 wks. from |
| | | | | baseline** |
| PD | Any | Yes or No | PD | no prior SD, PR or CR |
| Any | PD*** | Yes or No | PD | |
| Any | Any | Yes | PD | |

*See RECIST 1.1 manuscript for further details.
**Only for non-randomized trials with response as primary endpoint.
***In exceptional circumstances, unequivocal progression in non-target lesions may be accepted as disease progression.
Note:
Patients with a global deterioration of health status requiring discontinuation of treatment without objective evidence of disease progression at that time should be reported as “symptomatic deterioration.” Every effort should be made to document the objective progression even after discontinuation of treatment.

For patients with non-measurable disease (i.e., non-target disease), see Table 10.

TABLE 10

| Non-Target Lesions | New Lesions | Overall Response |
|---|---|---|
| CR | No | CR |
| Non-CR/non-PD | No | Non-CR/non-PD* |
| Not all evaluated | No | not evaluated |
| Unequivocal PD | Yes or No | PD |
| Any | Yes | PD |

*‘Non-CR/non-PD’ is preferred over ‘stable disease’ for non-target disease since SD is increasingly used as an endpoint for assessment of efficacy in some trials so to assign this category when no lesions can be measured is not advised

### Duration of Response

Duration of overall response: The duration of overall response is measured from the time measurement criteria are met for CR or PR (whichever is first recorded) until the first date that recurrent or progressive disease is objectively documented (taking as reference for progressive disease the smallest measurements recorded since the treatment started).

The duration of overall CR is measured from the time measurement criteria are first met for CR until the first date that progressive disease is objectively documented.

Duration of stable disease: Stable disease is measured from the start of the treatment until the criteria for progression are met, taking as reference the smallest measurements recorded since the treatment started, including the baseline measurements.

### Toxicity Criteria

The following adverse event management guidelines are intended to ensure the safety of each patient while on the study. The descriptions and grading scales found in the revised NCI Common Terminology Criteria for Adverse Events (CTCAE) version 5.0 will be utilized for AE reporting.
Related—There is a reasonable possibility that the study product caused the adverse event. Reasonable possibility means that there is evidence to suggest a causal relationship between the study product and the adverse event.
Not Related—There is not a reasonable possibility that the administration of the study product caused the event. s

Statistical Analyses

General Approach

Following a determination of safety and tolerability based on reporting toxicities, the fraction of all evaluable patients who experience a response will be reported along with confidence intervals.

Analysis of the Primary Endpoints

The toxicity grades and types per patient will be tabulated and reported.

The fraction of evaluable patients who experience a response (PR+CR) will be reported along with a 95% two-sided confidence interval.

### Analysis of the Secondary Endpoints

PFS and OS will be determined using the Kaplan-Meier method, and the median PFS and OS will be reported along with 95% confidence intervals. s

Safety Analyses

The fraction of patients who experience a toxicity, by grade and type of toxicity, will be tabulated.

### Baseline Descriptive Statistics

Baseline demographic characteristics will be reported.

### Interim Analysis

As indicated in the two-stage design, the number of responses after 9 evaluable patients have been treated will be noted and will be used to determine if enrollment to the second stage of accrual may proceed.

Further Analysis: VB-111 adenovector level in the blood and tumor samples of patients treated with VB-111 will be measured by RT-PCR. Results will be analyzed using descriptive statistics including confidence intervals when appropriate. Any statistical tests performed for evaluation of exploratory objective will be done without formal adjustment for multiple comparisons, but in the context of the number of tests performed.

What is claimed:

1. A vector comprising a Fas-chimera gene operably linked to an endothelial cell-specific promoter for use in reducing the size or inhibiting the growth of a tumor or eliminating a tumor in a subject in need thereof, wherein (a) the subject is to be administered an effective dose of the vector and (b) the subject is to be administered an effective dose of an immune checkpoint inhibitor.

2. A vector comprising a Fas-chimera gene operably linked to an endothelial cell-specific promoter for use in treating a tumor or a metastasis thereof in a subject in need thereof, wherein (a) the subject is to be administered an effective dose of the vector and (b) the subject is to be administered an effective dose of an immune checkpoint inhibitor.

3. The vector for use of claim 1 or 2, wherein the tumor is derived from or associated with Leukemia, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, non-small cell lung cancer, primary brain tumors (including glioblastoma multiforme), gastrointestinal (GI) cancers (including but not limited to cancers of the esophagus, gallbladder, biliary tract, liver, pancreas, stomach, small intestine, large intestine, colon, rectum, and anus), malignant pancreatic insulanoma, malignant carcinoid, urinary bladder cancer, premalignant skin lesions, testicular cancer, lymphomas, thyroid cancer, papillary thyroid cancer, neuroblastoma, glioblastima multiforme, neuroendocrine cancer, genitourinary tract cancer, malignant hypercalcemia, cervical cancer, endometrial cancer, adrenal cortical cancer, prostate cancer, Müllerian cancer, ovarian cancer, peritoneal cancer, fallopian tube cancer, or uterine papillary serous carcinoma.

4. The vector for use of any one of claims 1-3, wherein the effective dose of the vector is to be administered in an amount of about 1×10¹⁰ to about 1×10¹⁶, about 1×10¹¹ to about 1×10¹⁵, about 1×10¹¹ to about 1×10¹⁶, about 1×10¹² to about 1×10¹⁵, about 1×10¹² to about 1×10¹⁶, about 1×10¹² to about 1×10¹⁴, about 5×10¹² to about 1×10¹⁶, about 5×10¹² to about 1×10¹⁵, about 5×10¹² to about 1×10¹⁴, about 1×10¹² to about 1×10¹³, or about 1×10¹³ to about 1×10¹⁴ virus particles.

5. The vector for use of any one of claims 1-4, wherein the effective dose of the vector is to be administered in an amount of about 1×10¹⁶, 1×10¹⁵, 1×10¹⁴, 5×10¹³, 4×10¹³, 3×10¹³, 2×10¹³, 1×10¹³, 9×10¹², 8×10¹², 7×10¹², 6×10¹², 5×10¹², 4×10¹², 3×10¹², 2×10¹², 1×10¹², 9×10¹¹, 8×10¹¹, 7×10¹¹, 6×10¹¹, 5×10¹¹, 4×10¹¹, 3×10¹¹, 2×10¹¹, 1×10¹¹, 9×10¹⁰, 8×10¹⁰, 7×10¹⁰, 6×10¹⁰, 5×10¹⁰, 4×10¹⁰, 3×10¹⁰, 2×10¹⁰, or 1×10¹⁰ virus particles.

6. The vector for use of any one of claims 1-5, wherein the vector and the immune checkpoint inhibitor are to be administered sequentially. The vector for use of any one of claims 1-6, wherein the vector is to be repeatedly administered.

8. The vector for use of claim 7, wherein the vector is to be repeatedly administered every day, once in about 2 days, once in about 3 days, once in about 4 days, once in about 5 days, once in about 6 days, once in about 7 days, once in about 2 weeks, once in about 3 weeks, once in about 4 weeks, once in about 5 weeks, once in about 6 weeks, once in about 7 weeks, once in about 2 months, or once in about 6 months.

9. The vector for use of any one of claims 1 to 8, wherein the immune checkpoint inhibitor is to be repeatedly administered.

10. The vector for use of claim 9 wherein the immune checkpoint inhibitor is to be repeatedly administered once in about 7 days, once in about 2 weeks, once in about 3 weeks, once in about 4 weeks, once in about 2 months, once in about 3 months, once in about 4 months, once in about 5 months, or once in about 6 months.

11. The vector for use of any one of claims 1-11, wherein the immune checkpoint inhibitor is a PD-1 antagonist to be administered at an effective amount of less than about 15 mg/kg, less than about 14 mg/kg, less than about 13 mg/kg, less than about 12 mg/kg, less than about 11 mg/kg, less than about 10 mg/kg, less than about 9 mg/kg, less than about 8 mg/kg, less than about 7 mg/kg, less than about 6 mg/kg, less than about 5 mg/kg, less than about 4 mg/kg, less than about 3 mg/kg, less than about 2 mg/kg, or less than about 1 mg/kg.

12. The vector for use of claim 11, wherein the PD-1 antagonist is to be administered at an effective amount of a flat dose between about 100 mg to about 600 mg, about 120 mg to about 500 mg, about 140 mg to about 460 mg, about 180 mg to about 420 mg, about 200 mg to about 380 mg, about 220 mg, to about 340 mg, about 230 mg to about 300 mg, or about 230 mg to about 260 mg.

13. The vector for use of claim 11 or 12, wherein the PD-1 antagonist is an antibody selected from the group consisting of nivolumab, pembrolizumab, camrelizumab, cemiplimab, sintilimab, and PDR001.

14. The vector for use of any one of claims 1-13, wherein the vector is to administered in combination with an effective dose of one or more chemotherapeutic agents.

15. The vector for use of claim 14, wherein the one or more chemotherapeutic agents is selected from the group consisting of Acivicin; Aclarubicin; Acodazole Hydrochloride;
Acronine; Adriamycin; Adozelesin; Aldesleukin; Alimta; Altretamine; Ambomycin;
Ametantrone Acetate; Aminoglutethimide; Amsacrine; Anastrozole; Anthramycin;
Asparaginase; Asperlin; Azacitidine; Azetepa; Azotomycin; Batimastat; Benzodepa;
Bicalutamide; Bisantrene Hydrochloride; Bisnafide Dimesylate; Bevacizumab, Bizelesin;
Bleomycin Sulfate; Brequinar Sodium; Bropirimine; Busulfan; Cactinomycin;
Calusterone; Caracemide; Carbetimer; Carboplatin; Carmustine (BiCNU); Carubicin Hydrochloride; Carzelesin; Cedefingol; Chlorambucil; Cirolemycin; Cisplatin;
Cladribine; Crisnatol Mesylate; Cyclophosphamide; Cytarabine; Dacarbazine;
Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate;
Duazomycin; Edatrexate; Eflornithine Hydrochloride; Elsamitrucin; Enloplatin;
Enpromate; Epipropidine; Epirubicin Hydrochloride; Erbulozole; Esorubicin Hydrochloride; Estramustine; Estramustine Phosphate Sodium; Etanidazole; Etoposide;
Etoposide Phosphate; Etoprine; Fadrozole Hydrochloride; Fazarabine; Fenretinide;
Floxuridine; Fludarabine Phosphate; Fluorouracil; Flurocitabine; Fosquidone; Fostriecin Sodium; Gemcitabine; Gemcitabine Hydrochloride; Gliadel® wafer; Hydroxyurea;
Idarubicin Hydrochloride; Ifosfamide; Ilmofosine; Interferon Alfa-2a; Interferon Alfa-2b;
Interferon Alfa-nl; Interferon Alfa-n3; Interferon Beta-I a; Interferon Gamma-I b;
Iproplatin; Irinotecan Hydrochloride; Lanreotide Acetate; Letrozole; Leuprolide Acetate;
Liarozole Hydrochloride; Lometrexol Sodium; Lomustine (CCNU); Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate;
Methotrexate Sodium; Metoprine; Meturedepa; Mitindomide; Mitocarcin; Mitocromin;
Mitogillin; Mitomalcin; Mitomycin; Mitosper; Mitotane; Mitoxantrone Hydrochloride;
Mycophenolic Acid; Nocodazole; Nogalamycin; Ormaplatin; Oxisuran; pazotinib;
Paclitaxel; Pegaspargase; Peliomycin; Pentamustine; Peplomycin Sulfate; Perfosfamide;
Pipobroman; Piposulfan; Piroxantrone Hydrochloride; Plicamycin; Plomestane; Porfimer Sodium; Porfiromycin; Prednimustine; Procarbazine Hydrochloride; Puromycin;
Puromycin Hydrochloride; Pyrazofurin; Riboprine; Rogletimide; Safingol; Safingol Hydrochloride; Semustine; Simtrazene; Sorafinib; Sparfosate Sodium; Sparsomycin;
Spirogermanium Hydrochloride; Spiromustine; Spiroplatin; Streptonigrin; Streptozocin;
Sulofenur; Sunitinib; Talisomycin; Taxol; Tecogalan Sodium; Tegafur; Teloxantrone Hydrochloride; Temoporfin; Temozolomide; Teniposide; Teroxirone; Testolactone;
Thiamiprine; Thioguanine; Thiotepa; Tiazofuirin; Tirapazamine; Topotecan Hydrochloride; Toremifene Citrate; Trestolone Acetate; Triciribine Phosphate;
Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate;
Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole;
Zeniplatin; Zinostatin; and Zorubicin Hydrochloride.

16. The vector for use of any one of claims 1-15, wherein the vector comprises, consists of, or consists essentially of SEQ ID NO: 19.

17. The vector for use of any one of claims 1-16, wherein the vector is an isolated virus having European Collection of Cell Cultures (ECACC) Accession Number 13021201.
